(19) United States
(12) Patent Application Publication
(10) Pub. No.: US 2012/0213772 A1
(43) Pub. Date: Aug. 23, 2012

(54) Metabolic biomarkers of Crohn's Disease

(75) Inventors: Janet R. Jansson, Walnut Creek, CA (US); Benjamin Willing, Vancouver (CA); Phillippe Schmitt-Kopplin, Freising (DE); Agnes Fekete, Neuherberg (DE); Marianna Lucio, Muenchen (DE)

(73) Assignee: THE REGENTS OF THE UNIVERSITY OF CALIFORNIA, Oakland, CA (US)

(21) Appl. No.: 13/350,507

(22) Filed: Jan. 13, 2012

Related U.S. Application Data
(63) Continuation of application No. PCT/US2010/042360, filed on Jul. 16, 2010.
(60) Provisional application No. 61/226,210, filed on Jul. 16, 2009.

(51) Int. Cl. A61K 39/395; A61K 31/451; A61K 31/655; A61K 31/609; A61P 1/00; A61K 31/4164; A61K 31/58; A61K 31/573; A61K 31/52; A61K 31/519; H01J 49/26; A61K 31/496

(52) U.S. Cl. 424/133.1; 250/282; 514/327; 514/150; 514/166; 514/253.08; 514/398; 514/174; 514/179; 514/263.2; 514/263.3; 514/249; 424/158.1

(57) ABSTRACT

The invention provides for a method for identifying a biomarker in a fecal sample of a subject in need of such identification comprising: determining whether a fecal sample collected from a subject comprises a biomarker.

### RELATED PATENT APPLICATIONS

This application claims priority as a continuation application of PCT International Patent Application No. PCT/US2010/042360, filed Jul. 16, 2010, which claims priority to U.S. Provisional Patent Application Ser. No. 61/226,210, filed Jul. 16, 2009, which are hereby incorporated by reference in their entireties.

### STATEMENT OF GOVERNMENTAL SUPPORT

The invention was made with government support under Grant No. HSHQDC-07-X-00170 awarded by the U.S. Department of Homeland Security, and Contract No. DE-AC02-05CH11231 awarded by the U.S. Department of Energy. The government has certain rights in the invention.

### FIELD OF THE INVENTION

This invention relates generally to biomarkers involved in the diagnosis and prognosis of Crohn's Disease.

### BACKGROUND OF THE INVENTION

Crohn's disease (CD) is an inflammatory bowel disease (IBD), characterized by chronic inflammation of the gastrointestinal tract. The exact etiology of CD is unknown, but both the host genotype and environmental factors play a role, and it is known that disease induction requires the presence of bacteria. No specific pathogen has been defined as a causative agent, but individuals with CD have an imbalance or ‘dysbiosis’ of their intestinal microbiota, or microbiome [1], [2]. Dysbiosis in turn leads to a breakdown in the détente relationship between the microbiome and the host immune system, through unknown mechanisms.

Diagnostic and monitoring tools for CD are currently inadequate. Numerous serological biomarkers have been proposed for the diagnosis of CD. However, for clinical applications, none of the current markers stand-alone and they are therefore used in conjunction and as a supplement to endoscopy. Therefore, more accurate tools are needed for early diagnosis of CD; in particular non-invasive approaches that can be used in place of endoscopy.

Our aim was to search for metabolic biomarkers of CD as evidence of microbial functions in the gut. Recent advances in nuclear magnetic resonance (NMR) and mass spectrometry (MS) have made it possible to simultaneously assess thousands of metabolites corresponding to the “metabolome” and to determine end-points of metabolic processes in living systems [3]. NMR (1H NMR Spectroscopy) has revealed gut microbial contributions to metabolite compositions in different body fluids, including blood [4], urine [5], [6] and fecal extracts [7], [8]; and the latter has revealed some metabolites that are correlated to CD [7].

Although the information provided using NMR has been very valuable it is still limited by low resolution and sensitivity that only enables the annotation and quantification of a limited number of lower molecular weight molecules. By contrast, ion cyclotron resonance-Fourier transform mass spectrometry (ICR-FT/MS) with an ultrahigh mass resolution enables differentiation of very subtle variations in thousands of mass signals, including higher molecular weight metabolites [9]. The combination of coupled metabolite separation technologies to spectrometry and spectroscopy enables a multidimensional approach for the structural identification of new metabolites as recently exemplified for markers of diabetes and early stage insulin resistance [10]. Therefore, in this study we used ICR-FT/MS with its high dynamic range and high mass accuracy (0.2 ppm) to obtain non-targeted profiles of elementary compositions in fecal samples obtained from individuals with Crohn's disease.

### SUMMARY OF THE INVENTION

The invention provides for a method for identifying the Crohn's disease (CD) phenotype of a subject in need of such identification comprising: identifying one or more biomarkers as described in Tables 1-4 in a fecal sample collected from a subject.

The invention provides for a method for identifying a biomarker in a fecal sample of a subject in need of such identification comprising: determining whether a fecal sample collected from a subject comprises a biomarker as described in Tables 1-4.

The invention provides for a non-invasive diagnostic tool based on identification of metabolites as biomarkers to differentiate Crohn's disease (CD) phenotypes in fecal samples. The method can comprise determining the mass-to-charge ratio (m/z) value of each metabolite in the fecal sample, and determining whether the m/z value of the metabolite is found in one of Tables 1-4.

In some embodiments of the invention, the method is a method for identifying the Crohn's disease (CD) phenotype of a subject in need of such identification comprising: identifying one or more biomarkers as described in Table 1 in a fecal sample collected from a subject. In some embodiments of the invention, the method is a method for identifying a biomarker in a fecal sample of a subject in need of such identification comprising: determining whether a fecal sample collected from a subject comprises a biomarker as described in Table 1.

In some embodiments of the invention, the method is a method for identifying the Crohn's disease (CD) phenotype of a subject in need of such identification comprising: identifying one or more biomarkers as described in Table 2 in a fecal sample collected from a subject. In some embodiments of the invention, the method is a method for identifying a biomarker in a fecal sample of a subject in need of such identification comprising: determining whether a fecal sample collected from a subject comprises a biomarker as described in Table 2. The biomarkers described in Table 2 are biomarkers that are elevated or increased in amount in a fecal sample of a patient suffering from colonic Crohn's disease (CCD) compared to the amount of the biomarker in a patient suffering from ICD or an individual not suffering from CD. The presence of elevated or increased levels of one or more biomarkers in the feces of a subject described in Table 2 indicates that the subject is likely to suffer from CCD.

In some embodiments of the invention, the method is a method for identifying the Crohn's disease (CD) phenotype of a subject in need of such identification comprising: identifying one or more biomarkers as described in Table 3 in a fecal sample collected from a subject. In some embodiments of the invention, the method is a method for identifying a biomarker in a fecal sample of a subject in need of such identification comprising: determining whether a fecal sample collected from a subject comprises a biomarker as described in Table 3. The biomarkers described in Table 3 are biomarkers that are elevated or increased in amount in the feces of a patient suffering from ileal Crohn's disease (ICD) compared to the amount of the biomarker in a patient suffering from CCD or an individual not suffering from CD. The presence of elevated or increased levels of one or more biomarkers in the feces of a subject described in Table 3 indicates that the subject is likely to suffer from ICD.

In some embodiments of the invention, the method is a method for identifying the Crohn's disease (CD) phenotype of a subject in need of such identification comprising: identifying one or more biomarkers as described in Table 4 in a fecal sample collected from a subject. In some embodiments of the invention, the method is a method for identifying a biomarker in the feces of a subject in need of such identification comprising: determining whether a fecal sample collected from a subject comprises a biomarker as described in Table 4. The biomarkers described in Table 4 are biomarkers that are elevated or increased in amount in the feces of an individual patient not suffering from CD compared to the amount of the biomarker in a patient suffering from ICD or CCD. The presence of elevated or increased levels of one or more biomarkers in the feces of a subject described in Table 4 indicates that the subject is likely not to suffer from CD, that is, the subject is likely not to suffer from either ICD or CCD.

In some embodiments, the method further comprises the step of collecting the fecal sample from the subject, where the collecting step occurs prior to the determining step. In some embodiments, the method further comprises the step of treating the subject for CCD or ICD

In some embodiments, the subject is a human subject who has Crohn's disease (CD), is diagnosed with CD, is suspected to have CD, or is likely to have CD. The subject can be a healthy individual that does not have CD.

In some embodiments, the method further comprises: providing a warning to the subject that the subject is suffering from CD, ICD or CCD. In some embodiments, the method further comprises: altering treatment of the subject so that the subject is treated for CD, ICD or CCD. In some embodiments, the method further comprises: treating the subject for CD, ICD or CCD.

The invention provides for a method of treating a subject for CD comprising: (a) identifying a subject having CD using a method of the present invention, and (b) administering a therapeutically effective amount of a drug suitable for treating and/or ameliorating the symptom(s) of CD to the subject, and/or performing surgery on the subject. Such suitable drugs include: an antidiarrheal medicine (such as loperamide), an aminosalicylates (such as sulfasalazine or mesalamine), an antibiotics (such as ciprofloxacin or metronidazole), a corticosteroid (such as budesonide or prednisone), an immunomodulator (such as azathioprine (AZA), 6-mercaptopurine (6-MP), or methotrexate), and a tumor necrosis factor (TNF) antagonist (such as infliximab or Remicade®).

### BRIEF DESCRIPTION OF THE DRAWINGS

The foregoing aspects and others will be readily appreciated by the skilled artisan from the following description of illustrative embodiments when read in conjunction with the accompanying drawings.

FIG. 1 shows (A) Score and loading scatter plot of PLS analysis (Q²(cum)=0.96, R²(Y)=0.95). (Blue=ICD, Red=CCD and Green=Healthy). The masses with the highest regression coefficients were considered as discriminant. Coordinates on the figure axes are x 108. (B) Example of a differentiating metabolite for ICD (assigned at m/z of 391.2853) that is up regulated in the ICD group but the structure is unknown. (C) Mass at m/z of 407.2802 corresponding to 3a, 7a, or 12a-trihydroxy-5β-cholanate within the bile acid biosynthesis pathway. The intensities in B and C were normalized.

FIG. 2 shows (A) KEGG pathways that discriminated the three groups: ICD CCD and healthy. The m/z were selected after the validation of PLS model; (B) Tyrosine metabolism pathway, the red metabolites were identified and present in the ICD group. Green shading refers to enzymes that were annotated in Bacteroides vulgatus.

FIG. 3 shows the similarity plot (using Jaccard's index) of (A) microbial composition based on binary terminal-restriction fragment length polymorphism (TRFLP) data and (B) ICR-FT/MS data, respectively, from fecal samples of individuals with ICD (blue), CCD (red) and healthy individuals (green). Individuals were numbered as previously defined (11). Boxes indicate twin pairs that share the most similar metabolic and microbial profiles. Note: metaproteome data from the same fecal samples for individuals 6a and 6b have recently been published [33].

FIG. 4 shows the PLS loading plot (A) where bacterial abundance defined the Y matrix and ICR-FT/MS data were plotted as predictors of differentiating bacteria based on their regression coefficients. Masses with the greatest regression coefficients for specific bacterial populations that were more abundant [B. ovatus (BO), B. vulgatus (BV), and Escherichia coli (EC)] and less abundant [Faecalibacterium prausnitzii (FP) and Bacteroides uniformis (BU)] in the feces of individuals with ileal Crohn's disease (ICD) compared to individuals with colonic Crohn's disease (CCD) and healthy (H) individuals are identified in the heat plot (B). The heat plot indicates the abundance of masses, the predicted metabolite, the bacteria that were positively correlated to that metabolite and whether the metabolite was positively (+) or negatively (−) associated with ICD. The clustering on the x-axis is according to disease and that on the y-axis is according to the relative abundances of the same bacterial populations selected in (A) and corresponding abbreviations are given on the first column to the right of the heat plot. Individuals on the x-axis are coded according to [17]. Each cell is colored based on the detected level of the predicted metabolite:

### DETAILED DESCRIPTION

Before the present invention is described, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges may independently be included or excluded in the range, and each range where either, neither or both limits are included in the smaller ranges is also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, the preferred methods and materials are now described. All publications mentioned herein are incorporated herein by reference to disclose and describe the methods and/or materials in connection with which the publications are cited.

It must be noted that as used herein and in the appended claims, the singular forms “a”, “and”, and “the” include plural referents unless the context clearly dictates otherwise. Thus, for example, reference to “a metabolite” includes a plurality of such metabolites, and so forth.

These and other objects, advantages, and features of the invention will become apparent to those persons skilled in the art upon reading the details of the invention as more fully described below.

The invention provides for a method for identifying a biomarker in the feces of a subject in need of such identification comprising: (a) collecting a fecal sample from the subject, and (b) determining whether the fecal sample comprises a biomarker as described in Table 1.

In some embodiments, the subject is a human subject who has Crohn's disease (CD), is diagnosed with CD, is suspected to have CD, or is likely to have CD. The human subject may have one or more signs or symptoms of CD. Symptoms of CD include gastrointestinal, systemic, and extraintestinal symptoms. Gastrointestinal symptom include abdominal pain, diarrhea, short bowel syndrome, bloody bowel movements, flatulence, bloating, intestinal discomfort, intestinal stenosis, vomiting, nausea, small bowel obstruction, primary sclerosing cholangitis, perianal discomfort, itchiness or pain around the anus, inflammation, fistulization or abscess around the anal area, anal fissure, aphthous ulcers, dysphagia, and/or upper abdominal pain. Systemic symptoms include growth failure/retardation in children, fever, abscess, weight loss, decreased food intake, and/or malabsorption of carbohydrates and/or lipids. Extraintestinal symptoms include uvietis, eye pain, episcleritis, vision loss, seronegative spondyloarthropathy, arthritis, enthesitis, ankylosing spondylitis, sacroiliitis, erythema nodosum, septal panniculitis, pyoderma gangrenosum, swelling of the lower legs, deep venous thrombosis, pyoderma gangrenosum, pulmonary embolism, autoimmune hemolytic anemia, clubbing, osteoporosis, seizures, stroke, myopathy, peripheral neuropathy, headache, and/or depression.

In some embodiments, the fecal sample can be collected from feces after defecation, such as a stool sample, or from feces obtained directly from the rectum of a subject.

In some embodiments, the determining step comprises identifying in the fecal sample comprises up to or at least 1, 2, 3, 4, 5, 6, 7, 8, 9, 10, 20, 30, 40, 50, 100, 500, or 1,000, or any range encompassed thereof, biomarkers described in Tables 1-4. In some embodiments, the determining step comprises the method described in Example 1. The biomarkers can be determined using one method know to those skilled in the art such as any diagnosis platform, including, but not limited to, microarray, lab on a chip and MS platforms. In some embodiments, the determining step further comprises correlating the identified biomarker(s) with the biomarkers to determine whether the subject has CCD and/or ICD.

In some embodiments, the biomarker is a metabolite as described in Table 1.

From the biomarkers described in Table 1, the following fecal metabolites correlate with CCD: 4-hydroxyphenyl-acetylglycine and (Z)/4-hydroxyphenyl-acetaldehyde-oxime. From the biomarkers described in Table 1, the following fecal metabolites correlate with ICD: tyrosine, tryptophan, phenylalanine, taurocholate, chenodeoxyglycocholate/glycochenodeoxycholate, oleic acid, stearic acid, palmitic acid, arachidonic acid, octadecatrienoic acid, and linoleic acid.

Tables 2-4 described biomarker determinants that are useful for identifying subjects that are suffering from CCD, ICD, or neither CCD nor ICD. Each biomarker is identified by a number which corresponds to the unique m/z value of each biomarker. The value of m/z is the mass/charge ratio, wherein m is the molecular weight (in Daltons) and charge of each biomarker as measured by Fourier-transform ion cyclotron resonance mass spectrometry (ICR-FT/MS) (as described in Example 1). With the m/z determined for each biomarker, one skilled in the art is able to determine the compound by searching the KEGG database (http://www.genome.jp/kegg/) or Metlin Metabolite database (http://metlin.scripps.edu/).

TABLE 2
List of biomarker determinants that correlate with CCD (each number
is the m/z ratio of a specific biomarker determinant).

| | | | | | |
|---|---|---|---|---|---|
| 194.0459 | 333.0494 | 497.2324 | 446.131 | 742.6002 | 191.4785 |
| 413.1639 | 359.0885 | 526.3361 | 1152.897 | 293.2883 | 160.2306 |
| 356.0888 | 267.0986 | 277.0929 | 710.49 | 585.7048 | 161.6174 |
| 526.3173 | 450.1194 | 275.0649 | 471.2041 | 154.1502 | 150.4598 |
| 319.1914 | 343.1299 | 718.9685 | 946.3099 | 318.0615 | 169.438 |
| 359.0772 | 631.1072 | 839.3403 | 559.3465 | 180.6654 | 231.0354 |
| 414.1673 | 623.1771 | 487.2733 | 292.492 | 168.8796 | 339.1555 |
| 427.2159 | 589.3415 | 739.323 | 521.7699 | 156.1949 | 179.0714 |
| 357.0921 | 399.0722 | 630.5486 | 556.7734 | 159.6549 | 205.1015 |
| 399.1846 | 474.1617 | 299.2565 | 877.9414 | 361.0547 | 150.8737 |
| 411.0809 | 491.135 | 1501.895 | 534.0933 | 327.2165 | 239.7655 |
| 415.1795 | 556.3362 | 1059.602 | 1310.294 | 167.3395 | 169.3724 |
| 195.0492 | 331.4935 | 502.0495 | 1243.096 | 443.1399 | 184.4793 |
| 551.3047 | 306.019 | 527.649 | 525.2006 | 345.0704 | 188.3136 |
| 527.3207 | 710.339 | 366.0617 | 415.2491 | 389.0863 | 281.0856 |
| 385.169 | 572.0662 | 598.2519 | 559.7276 | 730.4117 | 319.0459 |
| 457.0541 | 515.167 | 462.1613 | 396.1388 | 155.2514 | 173.3283 |
| 285.1707 | 262.0332 | 162.544 | 330.2423 | 409.3541 | 257.762 |
| 397.169 | 480.1623 | 902.5425 | 384.1453 | 656.4263 | 159.8694 |
| 445.1901 | 428.1465 | 276.2709 | 175.2914 | 213.9209 | 176.8743 |
| 413.2003 | 455.1671 | 629.2933 | 206.7764 | 161.5257 | 330.0829 |
| 373.0929 | 302.6334 | 306.1837 | 501.1794 | 163.5295 | 150.9988 |
| 317.1506 | 445.1252 | 431.1673 | 484.5598 | 294.9459 | 300.1672 |
| 387.1085 | 398.0718 | 489.0291 | 158.8122 | 284.7403 | 304.0262 |
| 320.1948 | 310.7926 | 1857.719 | 872.9771 | 155.3958 | 164.5501 |
| 407.1711 | 373.0677 | 940.3148 | 523.2232 | 165.8308 | 167.5928 |
| 567.2996 | 367.0824 | 473.051 | 531.0838 | 184.057 | 228.7289 |
| 222.0772 | 621.5441 | 526.3066 | 761.5735 | 421.4934 | 261.876 |
| 208.0615 | 276.2515 | 781.2984 | 1181.84 | 165.7565 | 152.028 |
| 399.1482 | 299.9891 | 387.2244 | 898.9876 | 161.8813 | 229.8808 |
| 428.2193 | 495.2097 | 162.0451 | 467.1711 | 165.8954 | 320.4936 |
| 194.0432 | 507.162 | 158.0905 | 169.1729 | 650.7655 | 151.0508 |
| 456.1777 | 492.0935 | 950.7893 | 716.6075 | 165.8986 | 154.5585 |
| 415.1597 | 395.0775 | 224.6253 | 159.1038 | 363.1293 | 163.0578 |
| 360.0806 | 443.1922 | 240.0777 | 398.0734 | 355.1953 | 176.5339 |
| 475.2338 | 336.0805 | 566.2467 | 670.3046 | 158.9044 | 334.3821 |
| 495.2785 | 646.2764 | 890.2039 | 1097.469 | 274.7071 | 161.1207 |
| 429.1952 | 609.3233 | 1979.499 | 382.5124 | 371.1245 | 160.8288 |
| 236.0564 | 1990.579 | 653.089 | 1265.043 | 247.671 | 203.4437 |
| 415.2159 | 473.0596 | 939.5253 | 962.682 | 184.1798 | 155.9225 |
| 400.188 | 354.104 | 517.2064 | 162.1721 | 205.5513 | 162.7833 |
| 321.2071 | 477.2403 | 466.1226 | 596.2221 | 262.6351 | 263.2861 |
| 524.3017 | 521.1714 | 356.0349 | 821.7238 | 661.4055 | 203.7983 |
| 517.2443 | 540.1754 | 359.1502 | 611.0363 | 662.5312 | 167.4687 |
| 314.0047 | 281.0891 | 832.331 | 369.1504 | 152.9487 | 170.0277 |
| 555.1695 | 207.197 | 245.0819 | 575.731 | 170.7139 | 202.1752 |
| 412.0843 | 574.2136 | 1565.192 | 484.1659 | 157.5732 | 160.4124 |
| 447.2057 | 590.2793 | 434.7455 | 543.3135 | 328.3112 | 152.036 |
| 416.1829 | 311.0884 | 1769.501 | 1169.838 | 156.6496 | 195.0077 |
| 180.0302 | 310.8054 | 1517.076 | 769.8455 | 328.0673 | 207.1995 |
| 383.1533 | 194.0477 | 248.6087 | 552.2059 | 163.2841 | 167.1401 |
| 411.1482 | 500.1278 | 404.3231 | 497.2517 | 680.7551 | 155.3284 |
| 427.1795 | 565.1354 | 582.3428 | 807.2765 | 288.8339 | 216.2137 |
| 356.0704 | 497.2252 | 1810.896 | 533.3368 | 165.3608 | 167.9465 |
| 443.1745 | 452.0289 | 162.3865 | 464.0733 | 152.2059 | 342.2962 |
| 542.3122 | 292.4974 | 439.0432 | 668.3775 | 667.1789 | 163.9614 |
| 235.0724 | 561.1978 | 879.4108 | 163.0442 | 162.3715 | 299.3175 |
| 531.2997 | 497.1889 | 649.2885 | 601.8606 | 305.502 | 160.7473 |
| 316.0826 | 505.1938 | 261.7136 | 929.5721 | 187.6427 | 258.1325 |
| 194.0406 | 535.182 | 484.1358 | 873.8396 | 423.8364 | 262.1052 |
| 378.0707 | 529.2113 | 527.9937 | 531.2293 | 422.7882 | 238.8584 |
| 497.2578 | 428.0987 | 976.0043 | 1144.808 | 393.187 | 170.8448 |
| 415.1706 | 339.0873 | 494.1893 | 1256.695 | 285.1553 | 177.5926 |
| 446.1935 | 216.202 | 625.1613 | 527.3002 | 331.4894 | 246.0838 |
| 413.0878 | 271.9942 | 560.1415 | 151.2863 | 402.7992 | 238.5975 |
| 386.1723 | 207.1912 | 659.1624 | 1051.073 | 162.3858 | 184.3391 |
| 358.0932 | 412.1878 | 154.8154 | 459.114 | 310.7594 | 161.0352 |
| 569.3154 | 713.5001 | 616.6581 | 149.0048 | 343.2489 | 167.677 |
| 300.0878 | 1988.593 | 514.289 | 561.1732 | 165.6535 | 233.7378 |
| 293.1143 | 469.1867 | 651.3987 | 809.2546 | 230.0648 | 196.3669 |
| 458.1933 | 423.1694 | 617.4842 | 292.5234 | 400.0772 | 154.6638 |
| 479.2474 | 535.1643 | 310.7713 | 662.6204 | 345.4989 | 268.0563 |
| 390.1275 | 499.1719 | 497.2666 | 157.3789 | 178.6787 | 157.4481 |
| 356.0796 | 572.1495 | 259.1468 | 161.7884 | 371.9634 | 170.3808 |
| 433.1537 | 481.1981 | 527.2805 | 509.2019 | 155.3266 | 165.1745 |
| 149.9834 | 438.1556 | 568.8885 | 730.9607 | 370.2702 | 165.5707 |
| 236.0928 | 429.0942 | 268.0462 | 156.2134 | 160.9825 | 183.5402 |
| 224.0564 | 373.1043 | 834.5676 | 442.3213 | 166.4646 | 163.2131 |
| 385.1326 | 411.0568 | 601.3126 | 293.0779 | 355.1717 | 206.6286 |
| 398.1723 | 300.8662 | 303.0598 | 554.2939 | 382.1297 | 212.5602 |
| 238.0721 | 456.1145 | 1138.599 | 161.084 | 156.1685 | 180.6246 |
| 313.0466 | 412.0875 | 639.1731 | 665.2904 | 161.2922 | 289.4039 |
| 528.324 | 504.9017 | 470.2984 | 152.3623 | 240.4137 | 210.5655 |
| 366.1194 | 371.1077 | 157.0953 | 438.8665 | 301.4311 | 209.418 |
| 358.0955 | 430.2306 | 159.4468 | 521.9427 | 154.6703 | 163.6659 |
| 717.2223 | 480.1988 | 458.1302 | 163.5171 | 369.0918 | 156.002 |
| 430.1144 | 417.1753 | 482.1568 | 409.9361 | 318.9078 | 325.4509 |
| 401.1639 | 241.5284 | 552.964 | 474.0883 | 265.908 | 207.015 |
| 414.2036 | 355.1911 | 555.2001 | 549.3041 | 388.1824 | 209.9233 |
| 459.246 | 283.994 | 525.4365 | 581.8388 | 156.2651 | 174.0827 |
| 595.1854 | 274.0721 | 1151.926 | 385.1985 | 697.5869 | 326.987 |
| 412.1038 | 238.0179 | 1032.716 | 671.1703 | 195.0503 | 165.9423 |
| 427.1432 | 496.2631 | 429.1207 | 781.1479 | 164.0008 | 270.2255 |
| 568.303 | 662.5321 | 1594.61 | 653.0719 | 186.9429 | 309.9993 |
| 497.1276 | 659.2192 | 539.2123 | 621.3323 | 374.8057 | 254.5772 |
| 263.0925 | 320.1253 | 382.4951 | 463.0701 | 202.4825 | 164.1286 |
| 516.2675 | 710.3439 | 533.2461 | 265.0354 | 165.7466 | 192.3525 |
| 334.0932 | 184.3218 | 639.1168 | 362.1692 | 316.0779 | 162.6094 |
| 210.0772 | 629.1191 | 612.8651 | 828.6507 | 164.7441 | 161.1442 |
| 370.1144 | 271.0611 | 1536.469 | 531.1615 | 366.6135 | 172.1524 |
| 430.1985 | 161.6498 | 1146.37 | 772.5112 | 381.6323 | 228.8489 |
| 318.0983 | 555.1979 | 414.123 | 463.1536 | 204.488 | 299.5293 |
| 330.0983 | 292.5141 | 430.2388 | 600.1904 | 162.6024 | 164.2052 |
| 476.237 | 412.1118 | 226.0112 | 1147.724 | 229.9253 | 200.6931 |
| 606.2741 | 236.8196 | 495.2057 | 427.1894 | 147.9262 | 162.2479 |
| 298.0932 | 310.0569 | 602.6477 | 391.1243 | 415.1959 | 299.1608 |
| 296.0776 | 348.07 | 1492.496 | 1225.875 | 385.1477 | 167.0515 |
| 356.1069 | 543.2211 | 992.2986 | 413.0966 | 406.2348 | 196.3 |
| 510.3224 | 398.0685 | 257.0673 | 496.1964 | 163.7931 | 189.128 |
| 573.1239 | 246.0408 | 431.0751 | 587.3757 | 159.2998 | 213.917 |
| 472.1613 | 467.1072 | 772.5121 | 634.8036 | 251.1036 | 163.3386 |
| 194.0402 | 161.1515 | 1030.377 | 482.285 | 158.2429 | 165.7535 |
| 468.1299 | 382.1044 | 513.226 | 946.492 | 262.4148 | 318.1059 |
| 525.305 | 303.1238 | 299.2611 | 589.2177 | 321.5328 | 207.2074 |
| 438.167 | 481.2692 | 1147.087 | 640.1032 | 276.2333 | 153.4321 |
| 397.0653 | 283.0684 | 160.4291 | 520.6072 | 158.991 | 149.954 |
| 307.9992 | 1994.952 | 657.2183 | 574.1566 | 311.0558 | 327.053 |
| 158.6234 | 160.2201 | 1254.839 | 794.2422 | 255.4992 | 157.2601 |
| 295.0935 | 255.0662 | 341.1089 | 935.7201 | 163.5393 | 166.2291 |
| 194.5368 | 162.1728 | 1850.779 | 322.1364 | 246.2624 | 150.6879 |
| 457.1809 | 499.033 | 377.0133 | 216.6842 | 162.3958 | 309.1013 |
| 196.0501 | 355.0493 | 177.5985 | 410.4021 | 230.1393 | 160.4096 |
| 292.0826 | 253.0829 | 1690.878 | 569.9507 | 396.2003 | 154.6339 |
| 1992.809 | 226.0149 | 161.8731 | 506.1455 | 162.1612 | 160.6111 |
| 281.0779 | 331.0605 | 503.3871 | 470.1941 | 375.3513 | 290.7431 |
| 286.1741 | 159.1554 | 197.4053 | 281.4017 | 387.2188 | 204.2491 |
| 161.1144 | 216.1969 | 157.2398 | 701.1952 | 230.4029 | 156.8399 |
| 357.0858 | 194.5396 | 1482.413 | 593.3417 | 167.672 | 177.8433 |
| 220.0615 | 195.03 | 552.5009 | 482.8968 | 196.3086 | 167.7583 |
| 478.1355 | 216.2062 | 414.3764 | 155.3947 | 157.7161 | 184.6925 |
| 471.2249 | 405.2858 | 1659.081 | 904.2758 | 222.0737 | 165.74 |
| 374.0963 | 236.7858 | 497.2721 | 457.4576 | 165.2127 | 243.0798 |
| 518.2476 | 354.167 | 620.3902 | 951.5481 | 336.0273 | 167.8572 |
| 318.154 | 311.0806 | 414.0679 | 302.3268 | 265.1092 | 262.3711 |
| 194.0486 | 464.2654 | 457.422 | 424.8357 | 338.1237 | 285.1691 |
| 496.2819 | 487.1974 | 813.4058 | 819.2493 | 329.8989 | 165.9832 |
| 417.1589 | 540.1944 | 211.6224 | 154.0064 | 178.5232 | 161.1628 |
| 250.1085 | 452.0344 | 1397.119 | 506.2258 | 180.0279 | 223.8882 |
| 441.2143 | 570.1858 | 488.2232 | 734.0054 | 154.7339 | 147.9554 |
| 495.1662 | 160.1261 | 487.2157 | 164.6725 | 175.8937 | 167.6377 |
| 285.0616 | 159.9872 | 165.7428 | 169.3895 | 355.1299 | 160.4467 |
| 388.1119 | 313.1023 | 459.2069 | 182.2697 | 160.4101 | 302.1534 |
| 400.1038 | 147.5171 | 549.2811 | 261.7295 | 307.0836 | 164.8783 |
| 276.2564 | 290.0671 | 331.4822 | 978.63 | 293.0878 | 271.0143 |
| 350.0881 | 1988.487 | 161.8835 | 162.7623 | 194.4444 | 179.0562 |
| 323.0884 | 498.2094 | 642.1322 | 475.22 | 380.1325 | 172.2327 |
| 408.1745 | 394.1588 | 674.6271 | 423.6551 | 160.6945 | 161.4033 |
| 426.1194 | 457.2093 | 793.8414 | 230.9996 | 153.1269 | 188.1503 |
| 353.1006 | 157.7916 | 1064.524 | 655.4983 | 223.6816 | 198.6569 |
| 427.1309 | 298.0098 | 382.5321 | 437.091 | 164.6018 | 161.9103 |
| 543.3155 | 160.3874 | 1771.697 | 743.8233 | 243.3427 | 203.4763 |
| 461.185 | 292.5011 | 434.7407 | 558.8916 | 357.0963 | 175.2306 |
| 356.098 | 422.2548 | 168.15 | 869.3863 | 162.4477 | 314.3302 |
| 404.0987 | 475.2003 | 1503.445 | 292.5002 | 158.0525 | 196.6616 |
| 427.1835 | 253.0288 | 495.1719 | 605.262 | 162.5823 | 151.1538 |
| 349.071 | 1995.015 | 1755.556 | 803.8216 | 169.177 | 228.5143 |
| 189.0207 | 163.1626 | 160.6253 | 878.2281 | 220.1975 | 167.8626 |
| 429.1667 | 1995.042 | 160.3909 | 164.5962 | 169.6406 | 161.8311 |
| 291.0986 | 248.6275 | 1536.693 | 594.1619 | 288.9929 | 223.7878 |
| 351.1197 | 239.0595 | 526.5962 | 497.2158 | 152.798 | 164.453 |
| 519.2599 | 1988.649 | 161.3026 | 440.0991 | 160.842 | 206.2807 |
| 497.1088 | 552.4586 | 1217.382 | 331.0836 | 166.4604 | 190.2152 |
| 527.6681 | 202.4874 | 248.6347 | 585.3625 | 166.5638 | 281.0674 |
| 307.1299 | 432.2471 | 957.4182 | 394.1018 | 159.5857 | 157.8324 |
| 403.151 | 616.2244 | 359.0746 | 790.7355 | 177.587 | 156.4365 |
| 441.1952 | 831.3266 | 220.5072 | 1115.255 | 382.1264 | 213.0543 |
| 440.1826 | 391.2776 | 883.1026 | 164.3337 | 167.6682 | 303.1812 |
| 395.1533 | 531.1232 | 163.3867 | 156.7595 | 342.9564 | 201.1632 |
| 425.0966 | 570.205 | 965.1779 | 581.1479 | 317.9001 | 311.0298 |
| 442.1143 | 1990.8 | 373.2742 | 514.6999 | 164.8642 | 160.5973 |
| 265.1082 | 495.9845 | 429.6297 | 472.649 | 365.4203 | 300.6565 |
| 531.0398 | 692.2687 | 489.2544 | 204.8126 | 239.0526 | 263.8446 |
| 492.9841 | 429.1554 | 428.1065 | 258.1329 | 317.9916 | 174.8572 |
| 434.291 | 543.1928 | 614.15 | 212.2418 | 210.63 | 323.4244 |
| 284.0928 | 736.1697 | 1120.757 | 520.2996 | 209.5986 | 165.629 |
| 400.1516 | 1992.753 | 159.8381 | 509.067 | 299.129 | 175.3038 |
| 814.2985 | 437.935 | 634.9445 | 610.572 | 160.5772 | 195.4334 |
| 308.0776 | 977.354 | 436.14 | 396.1994 | 163.2815 | 163.122 |
| 452.1074 | 517.1229 | 1803.888 | 1089.619 | 236.1646 | 209.8416 |
| 184.3182 | 1243.034 | 156.7608 | 151.7372 | 155.3399 | 247.0512 |
| 443.2511 | 576.112 | 621.2611 | 164.8021 | 297.0769 | 170.7307 |
| 494.9811 | 544.1225 | 156.8914 | 164.4995 | 167.9859 | 321.9431 |
| 415.1431 | 357.1108 | 155.359 | 791.3774 | 213.9549 | 159.9835 |
| 207.0775 | 585.1965 | 401.8373 | 160.0187 | 367.8979 | 153.0694 |
| 250.0721 | 249.0881 | 532.1537 | 517.151 | 148.679 | 170.9266 |
| 320.0776 | 510.2789 | 1531.844 | 635.3574 | 217.2987 | 155.3998 |
| 455.1017 | 584.359 | 438.6592 | 476.2419 | 364.1929 | 266.2662 |
| 608.3205 | 451.1761 | 521.6748 | 711.4016 | 149.4978 | 223.6163 |
| 416.0987 | 493.1338 | 456.1622 | 752.1628 | 172.1977 | 152.531 |
| 355.0312 | 310.5313 | 972.0708 | 342.2395 | 166.1374 | 168.4404 |
| 292.5075 | 1994.944 | 570.1982 | 374.2217 | 158.1254 | 185.8548 |
| 613.1563 | 495.1231 | 565.1716 | 307.046 | 159.7296 | 166.8317 |
| 280.0827 | 417.2839 | 402.083 | 419.1937 | 385.9424 | 320.1572 |
| 196.0616 | 510.104 | 1040.966 | 705.9789 | 167.9769 | 160.8561 |
| 472.1248 | 392.2077 | 166.7977 | 491.2143 | 212.931 | 245.7515 |
| 262.0721 | 619.1726 | 659.6982 | 922.8248 | 166.2231 | 158.2057 |
| 380.1827 | 744.361 | 171.4739 | 207.613 | 162.5449 | 180.4853 |
| 527.6278 | 496.1251 | 930.2676 | 494.0617 | 196.5208 | 179.8534 |
| 418.162 | 396.0701 | 873.617 | 499.367 | 284.5648 | 161.3568 |
| 402.2037 | 595.1063 | 544.2727 | 542.2141 | 214.6669 | 164.0028 |
| 340.1514 | 365.1274 | 779.1301 | 496.1246 | 150.6868 | 165.7642 |
| 530.283 | 389.1946 | 207.1891 | 498.2569 | 264.1927 | 231.8159 |
| 252.0513 | 428.9845 | 1476.726 | 887.8181 | 365.0643 | 236.8682 |
| 209.0648 | 593.2366 | 1062.942 | 977.4285 | 170.9021 | 159.2735 |
| 184.3191 | 1034.393 | 198.1669 | 206.9375 | 168.5688 | 171.936 |
| 312.0725 | 248.0927 | 1517.885 | 160.1889 | 160.0982 | 205.4401 |
| 611.1803 | 522.2862 | 1554.502 | 495.1162 | 244.9511 | 147.716 |
| 386.1093 | 872.3406 | 292.516 | 453.1278 | 152.1957 | 161.862 |
| 266.0669 | 554.1539 | 1535.095 | 450.6381 | 164.599 | 276.5954 |
| 223.0805 | 487.3057 | 558.2802 | 840.1292 | 212.603 | 220.0075 |
| 352.1515 | 440.0986 | 1294.354 | 546.9425 | 163.1976 | 234.2377 |
| 194.0513 | 1992.959 | 248.6096 | 494.476 | 156.5887 | 167.8195 |
| 526.2972 | 444.1736 | 562.2891 | 362.499 | 149.0847 | 260.6862 |
| 1992.783 | 331.0935 | 414.1718 | 522.2046 | 151.6277 | 164.2282 |
| 196.0526 | 1992.847 | 349.1039 | 185.9112 | 165.1773 | 325.0069 |
| 284.0776 | 1992.899 | 602.7422 | 316.902 | 160.0505 | 147.9784 |
| 452.1351 | 466.2809 | 549.1409 | 1060.322 | 159.1382 | 171.1477 |
| 470.1933 | 208.0808 | 438.083 | 365.7948 | 384.8071 | 174.545 |
| 370.1044 | 586.3207 | 1528.434 | 307.4776 | 155.3201 | 169.6065 |
| 312.099 | 559.1081 | 873.1314 | 469.9278 | 163.2787 | 170.8516 |
| 216.2041 | 426.0998 | 1008.553 | 536.1926 | 222.4169 | 174.3266 |
| 281.1143 | 371.1169 | 343.0864 | 165.8273 | 171.1624 | 224.3989 |
| 373.1405 | 1675.25 | 520.0116 | 648.9904 | 165.5875 | 188.0885 |
| 434.1569 | 456.1051 | 587.2897 | 250.2379 | 300.2601 | 263.7511 |
| 470.1303 | 383.0441 | 305.4976 | 828.1328 | 341.2288 | 147.5456 |
| 195.0465 | 650.3002 | 563.517 | 158.2328 | 216.6629 | 207.5083 |
| 462.1882 | 208.0584 | 355.2025 | 958.7179 | 347.503 | 148.6634 |
| 322.2105 | 554.1304 | 224.6004 | 598.1567 | 314.1145 | 170.3934 |
| 310.7746 | 480.0191 | 952.5964 | 497.248 | 349.0564 | 167.036 |
| 412.2058 | 310.7864 | 452.0518 | 432.1931 | 171.6148 | 150.9352 |
| 495.2421 | 184.3497 | 478.2307 | 575.2871 | 161.8764 | 170.48 |
| 315.1449 | 597.1926 | 161.5789 | 872.2483 | 156.9093 | 160.265 |
| 276.0878 | 309.0728 | 833.3412 | 164.9708 | 194.0788 | 160.8279 |
| 409.1325 | 452.0216 | 310.7689 | 235.0647 | 256.342 | 212.3574 |
| 439.1123 | 315.0387 | 1462.685 | 167.2599 | 154.8645 | 174.9202 |
| 1990.606 | 613.1707 | 436.1513 | 390.2231 | 163.3214 | 159.0447 |
| 175.0612 | 1995.054 | 593.3219 | 553.3219 | 198.3798 | 153.2204 |
| 710.3617 | 372.0474 | 551.3107 | 528.211 | 308.5622 | 229.9857 |
| 433.9635 | 549.2441 | 170.452 | 401.0985 | 207.6926 | 163.0842 |
| 332.0776 | 578.1277 | 1405.53 | 154.4091 | 152.0016 | 241.4191 |
| 369.0704 | 356.0613 | 310.7761 | 1101.684 | 184.2048 | 321.7265 |
| 510.1405 | 382.5248 | 440.2006 | 473.0211 | 161.1199 | 206.3161 |
| 326.0881 | 418.2962 | 390.0705 | 700.9804 | 353.4524 | 173.111 |
| 295.13 | 480.1752 | 565.1709 | 164.5547 | 160.1748 | 169.7344 |
| 540.2966 | 216.1888 | 497.2412 | 171.9516 | 204.4273 | 220.9177 |
| 498.0295 | 192.9794 | 455.2101 | 395.4237 | 184.0223 | 266.9319 |
| 427.199 | 1988.537 | 420.1413 | 226.0126 | 163.2337 | 310.8011 |
| 302.1033 | 452.0465 | 1503.585 | 408.0184 | 165.8535 | 163.1371 |
| 573.2105 | 519.2509 | 591.4436 | 1055.014 | 202.1166 | 157.6839 |
| 374.0881 | 628.256 | 160.793 | 842.3291 | 174.7646 | 298.4427 |
| 615.1719 | 1990.859 | 634.6973 | 165.5766 | 319.0602 | 167.6747 |
| 395.0442 | 232.0284 | 162.9405 | 276.2284 | 164.3052 | 161.3422 |
| 507.26 | 951.5138 | 393.1092 | 541.2046 | 151.7659 | 268.253 |
| 514.1355 | 488.2725 | 516.2871 | 514.2202 | 170.4424 | 206.1209 |
| 226.0273 | 285.0961 | 421.1302 | 515.3083 | 190.7866 | 225.1572 |
| 245.0125 | 507.2365 | 877.9917 | 454.5668 | 169.86 | 207.161 |
| 195.0429 | 599.2125 | 276.2321 | 159.6445 | 155.1827 | 285.598 |
| 291.0509 | 334.2024 | 414.3913 | 961.8413 | 313.6312 | 160.5828 |
| 539.1912 | 615.2183 | 178.8137 | 746.4771 | 160.409 | 149.0778 |
| 478.1595 | 396.0106 | 528.6679 | 1118.936 | 239.6385 | 179.6058 |
| 236.794 | 414.3691 | 927.9309 | 167.4294 | 356.8759 | 163.1367 |
| 471.2824 | 710.3341 | 1593.637 | 445.2269 | 168.9396 | 170.294 |
| 596.1888 | 518.1037 | 1480.424 | 233.1295 | 167.2372 | 181.6016 |
| 526.2773 | 368.4238 | 647.2797 | 364.3507 | 160.7253 | 156.2841 |
| 313.0499 | 452.0564 | 159.9074 | 773.2225 | 217.2685 | 184.17 |
| 439.034 | 160.0533 | 375.0983 | 423.1142 | 166.3332 | 154.1746 |
| 315.072 | 622.2692 | 621.2623 | 438.8787 | 213.8133 | 147.7317 |
| 306.07 | 339.0389 | 661.6254 | 193.521 | 151.6782 | 207.3619 |
| 589.1563 | 554.1544 | 413.1704 | 659.5799 | 244.3953 | 167.3547 |
| 384.1567 | 617.2617 | 165.6181 | 334.3552 | 159.145 | 278.3841 |
| 1990.701 | 425.1851 | 276.2345 | 589.9203 | 350.615 | 161.2105 |
| 484.2048 | 164.0354 | 375.1062 | 439.7995 | 171.8653 | 201.5613 |
| 628.1161 | 382.4972 | 741.337 | 477.2376 | 158.7033 | 306.6723 |
| 255.1503 | 224.012 | 405.3038 | 501.3468 | 156.5451 | 156.2268 |
| 479.2286 | 230.0128 | 545.2944 | 485.1121 | 340.0826 | 167.5623 |
| 379.074 | 1988.433 | 310.7131 | 871.1613 | 270.5661 | 240.7014 |
| 710.3541 | 541.2137 | 662.4185 | 634.4127 | 161.4125 | 171.4606 |
| 378.1557 | 649.2814 | 519.1301 | 888.7402 | 319.2127 | 233.5479 |
| 178.051 | 710.3448 | 1028.673 | 222.3497 | 235.4093 | 229.1562 |
| 282.0619 | 413.1586 | 653.7236 | 231.9707 | 160.4956 | 151.5342 |
| 371.0441 | 441.186 | 530.2373 | 416.1336 | 210.8348 | 228.6281 |
| 252.0045 | 637.1686 | 474.0897 | 150.6082 | 168.7304 | 188.9404 |
| 556.1729 | 533.1256 | 755.1924 | 538.0465 | 160.0253 | 165.2966 |
| 487.22 | 743.3526 | 459.2021 | 543.6659 | 161.8698 | 274.0718 |
| 185.0456 | 465.139 | 737.8562 | 703.1876 | 356.1533 | 151.9184 |
| 202.0721 | 382.4979 | 937.0655 | 523.2218 | 176.5789 | 149.6682 |
| 420.1665 | 243.0298 | 1055.462 | 828.7464 | 167.2155 | 148.4392 |
| 421.1253 | 305.0741 | 166.5275 | 684.8928 | 237.3685 | 198.1496 |
| 476.0476 | 817.2754 | 558.3143 | 563.8472 | 246.3684 | 223.5652 |
| 424.1331 | 524.273 | 261.7313 | 949.8769 | 162.4617 | 168.1794 |
| 160.4362 | 368.6962 | 1163.203 | 438.0835 | 212.7746 | 165.754 |
| 536.236 | 291.1272 | 449.1885 | 864.2679 | 163.2774 | 222.6381 |
| 382.5158 | 442.0101 | 433.2897 | 751.5598 | 279.9552 | 183.2899 |
| 539.2433 | 161.4263 | 597.2784 | 858.3611 | 264.3264 | 233.558 |
| 485.185 | 169.0089 | 662.3116 | 548.1545 | 282.306 | 260.4462 |
| 248.6286 | 507.114 | 505.1498 | 331.0239 | 166.5379 | 164.2632 |
| 566.1703 | 310.7969 | 536.151 | 305.1059 | 173.5666 | 284.8021 |
| 467.1782 | 410.0967 | 226.0375 | 168.3546 | 281.0848 | 160.8248 |
| 233.082 | 292.4855 | 399.3561 | 922.3063 | 158.1877 | 164.9105 |
| 607.2776 | 413.1006 | 274.0355 | 371.1617 | 161.6995 | 150.483 |
| 382.5069 | 700.3886 | 543.4208 | 636.5761 | 218.4246 | 244.0712 |
| 194.0481 | 613.0966 | 1131.203 | 645.7621 | 372.6547 | 152.3814 |
| 459.1965 | 451.2893 | 679.2353 | 657.8998 | 226.046 | 266.9483 |
| 178.0874 | 282.0812 | 1408.103 | 774.5557 | 365.0504 | 197.2319 |
| 429.1039 | 559.2316 | 956.1397 | 1010.42 | 162.3208 | 196.0587 |
| 532.303 | 195.0435 | 654.0797 | 555.0102 | 157.1279 | 151.003 |
| 569.2017 | 551.1818 | 206.3926 | 387.9794 | 310.3902 | 203.4514 |
| 288.0513 | 193.9951 | 688.3232 | 602.2501 | 270.1095 | 254.423 |
| 261.7077 | 401.144 | 1006.09 | 177.5885 | 207.2476 | 245.3549 |
| 403.0767 | 385.2226 | 511.2159 | 514.2856 | 169.0867 | 167.9439 |
| 548.1774 | 454.9875 | 448.1037 | 156.7135 | 165.6503 | 170.359 |
| 357.0742 | 665.1721 | 1248.649 | 585.6771 | 314.9143 | 171.4765 |
| 386.1359 | 328.0203 | 461.2783 | 353.6345 | 302.1715 | 157.0509 |
| 415.9529 | 616.3508 | 156.6489 | 166.743 | 163.611 | 249.582 |
| 466.1143 | 398.0883 | 410.0881 | 163.9244 | 166.4801 | 274.1652 |
| 226.0239 | 632.314 | 451.0705 | 168.9794 | 163.273 | 165.8638 |
| 402.1275 | 253.1347 | 529.2152 | 469.9027 | 161.0463 | 243.0214 |
| 401.2406 | 511.2313 | 160.3281 | 753.9017 | 155.9569 | 150.5204 |
| 451.2198 | 355.1959 | 487.5491 | 446.2547 | 204.1451 | 216.2738 |
| 483.1144 | 670.1826 | 191.4844 | 161.6914 | 165.0416 | 178.3755 |
| 1990.649 | 1988.675 | 495.1654 | 428.3839 | 231.2734 | 243.7834 |
| 390.1673 | 488.1489 | 617.989 | 302.0752 | 199.4441 | 301.8028 |
| 557.294 | 579.2484 | 744.4686 | 543.8195 | 157.7955 | 263.8578 |
| 368.0888 | 1911.494 | 375.1296 | 453.2479 | 262.9548 | 169.4018 |
| 210.0408 | 439.0706 | 156.5679 | 310.5553 | 351.0487 | 154.7874 |
| 620.2902 | 230.066 | 528.1989 | 699.2891 | 158.3106 | 172.9408 |
| 247.0611 | 1769.538 | 558.2822 | 679.4988 | 378.8009 | 266.8005 |
| 355.1807 | 1994.912 | 995.0509 | 406.8018 | 204.1673 | 149.4564 |
| 278.0674 | 97.2616 | 902.9658 | 583.2744 | 178.2927 | 222.7535 |
| 448.2704 | 359.099 | 187.3008 | 542.1585 | 335.0919 | 207.5268 |
| 314.1034 | 306.1711 | 159.0113 | 497.1312 | 165.6892 | 169.4893 |
| 337.1042 | 331.4925 | 1010.729 | 686.791 | 174.7762 | 209.2474 |
| 236.0758 | 452.0556 | 162.5218 | 335.1493 | 159.5131 | 255.7732 |
| 264.1241 | 493.9874 | 695.1827 | 172.1482 | 248.5422 | 163.7701 |
| 355.1754 | 497.2555 | 452.1554 | 169.3499 | 385.068 | 164.7462 |
| 718.2259 | 463.1396 | 470.4674 | 379.0669 | 151.2656 | 165.9986 |
| 317.0779 | 1990.796 | 369.6582 | 431.9874 | 355.1661 | 252.0506 |
| 357.0957 | 441.0494 | 201.8268 | 157.2152 | 302.8296 | 252.3932 |
| 976.3508 | 734.2795 | 529.6081 | 608.414 | 164.0193 | 219.435 |
| 530.1664 | 591.1731 | 519.2515 | 693.6083 | 155.6025 | 205.8096 |
| 574.1272 | 310.1279 | 489.0891 | 171.4704 | 150.5819 | 147.4619 |
| 317.086 | 621.6465 | 1163.447 | 772.2878 | 328.1185 | 213.1649 |
| 327.0363 | 427.9579 | 207.2003 | 520.23 | 162.7153 | 168.1396 |
| 315.0081 | 167.4745 | 774.9111 | 660.7564 | 157.3998 | 196.1135 |
| 454.1144 | 292.5006 | 321.1014 | 478.9803 | 161.7157 | 148.8771 |
| 511.3257 | 505.1971 | 157.8024 | 547.9294 | 169.7542 | 201.6386 |
| 457.1901 | 522.1124 | 1337.736 | 500.2067 | 209.5736 | 269.5134 |
| 187.0071 | 248.624 | 237.0961 | 591.9985 | 213.0053 | 205.9498 |
| 466.1434 | 521.2448 | 395.0742 | 614.0924 | 339.421 | 196.0612 |
| 150.056 | 409.1364 | 468.1781 | 487.1838 | 335.3909 | 198.4803 |
| 450.286 | 565.2783 | 451.0881 | 629.8283 | 162.5368 | 268.3836 |
| 226.0357 | 414.3874 | 1145.455 | 676.3302 | 296.1252 | 209.1368 |
| 521.1414 | 261.6959 | 1219.451 | 163.2677 | 152.6383 | 201.9141 |
| 507.2461 | 415.1661 | 957.019 | 379.7543 | 251.9306 | 215.9852 |
| 508.1611 | 331.4792 | 162.0763 | 595.465 | 159.3379 | 163.0853 |
| 615.2213 | 699.8764 | 157.4828 | 381.56 | 207.6587 | 164.6496 |
| 410.1246 | 411.0729 | 165.3743 | 547.6389 | 309.0023 | 165.6434 |
| 301.0911 | 292.5174 | 161.7165 | 526.2516 | 322.4286 | 209.3055 |
| 815.3018 | 581.1921 | 1246.444 | 879.9418 | 161.9289 | 154.8365 |
| 194.5423 | 382.1019 | 248.6427 | 739.2352 | 198.1389 | 221.8353 |
| 357.1015 | 940.1552 | 504.2097 | 527.8981 | 154.2439 | 165.0366 |
| 425.0879 | 497.3308 | 318.4159 | 553.9992 | 171.1359 | 186.1096 |
| 742.3457 | 609.42 | 833.9873 | 347.8784 | 290.252 | 169.1457 |
| 436.234 | 148.4041 | 482.3212 | 378.5736 | 157.3714 | 163.6661 |
| 401.1803 | 331.476 | 632.4218 | 359.5922 | 184.1741 | 252.4669 |
| 335.0284 | 487.2354 | 491.1461 | 162.3072 | 271.5235 | 151.8566 |
| 560.2575 | 547.2182 | 166.636 | 164.8049 | 299.829 | 208.0932 |
| 514.2881 | 261.7112 | 500.1888 | 389.0991 | 168.8459 | 167.8598 |
| 331.1221 | 710.4041 | 500.1196 | 198.1592 | 212.4394 | 189.0456 |
| 195.0439 | 608.2698 | 496.1271 | 717.552 | 206.8734 | 269.0484 |
| 458.1093 | 355.1542 | 521.1156 | 153.1599 | 156.4283 | 167.1337 |
| 315.0987 | 407.1013 | 812.5876 | 534.3823 | 154.5772 | 169.0234 |
| 357.1091 | 235.0685 | 569.1675 | 565.1478 | 167.0592 | 218.7854 |
| 390.2652 | 479.1627 | 593.17 | 165.4973 | 156.4863 | 208.433 |
| 616.1494 | 621.4403 | 156.4086 | 223.0725 | 254.0866 | 212.5249 |
| 368.8353 | 370.0009 | 477.1911 | 697.5763 | 368.9783 | 156.5587 |
| 587.1772 | 898.8417 | 1032.754 | 307.5148 | 344.4772 | 161.7463 |
| 1994.996 | 524.2973 | 492.2345 | 454.1456 | 331.9142 | 193.2369 |
| 541.1563 | 456.1696 | 962.0437 | 438.1039 | 215.0826 | 157.5807 |
| 1990.669 | 442.162 | 165.3062 | 659.7979 | 159.8278 | 162.753 |
| 494.1092 | 638.445 | 504.2194 | 772.18 | 245.0221 | 164.564 |
| 236.788 | 324.0918 | 662.6594 | 358.1047 | 197.0006 | 153.5185 |
| 424.1126 | 355.2059 | 442.2057 | 223.0143 | 243.8901 | 169.7321 |
| 314.05 | 343.0936 | 473.1875 | 568.7912 | 167.8426 | 173.5421 |
| 370.0932 | 369.9967 | 468.6877 | 165.0396 | 165.0431 | 218.7784 |
| 483.1732 | 477.0861 | 310.7661 | 182.4135 | 156.3462 | 167.8665 |
| 377.2003 | 743.3489 | 533.2144 | 634.6275 | 167.2962 | 158.5583 |
| 520.2632 | 177.5957 | 526.5604 | 657.6149 | 149.9663 | 159.6447 |
| 1990.79 | 1994.907 | 218.0709 | 177.1931 | 209.0823 | 165.5497 |
| 416.2806 | 569.3712 | 579.2783 | 168.1432 | 163.1051 | 234.6594 |
| 271.0976 | 551.1593 | 1207.419 | 157.521 | 207.7953 | 222.4345 |
| 429.2117 | 456.1725 | 404.1461 | 162.2709 | 162.0052 | 198.343 |
| 397.0598 | 565.2139 | 1365.794 | 361.8577 | 255.3526 | 238.1159 |
| 357.1344 | 695.1629 | 345.1017 | 254.0664 | 165.6541 | 243.8916 |
| 444.1622 | 714.5032 | 471.0966 | 158.7949 | 159.2133 | 149.1476 |
| 569.2405 | 392.294 | 438.1125 | 162.0089 | 308.985 | 153.9591 |
| 251.0673 | 637.2032 | 156.2184 | 609.6188 | 226.6606 | 156.4788 |
| 359.0964 | 477.0513 | 460.8777 | 209.7301 | 173.3787 | 161.3601 |
| 292.5064 | 249.1132 | 533.6558 | 491.0233 | 167.8017 | 196.2243 |
| 571.182 | 425.1154 | 330.0617 | 360.1411 | 306.3519 | 160.7052 |
| 432.2755 | 455.252 | 575.4518 | 445.1849 | 298.1083 | 221.0302 |
| 236.7798 | 375.0826 | 159.6202 | 716.2256 | 335.0139 | 162.1796 |
| 566.1907 | 1894.675 | 579.3185 | 585.4224 | 277.9984 | 163.2366 |
| 445.1352 | 248.6165 | 610.3707 | 476.0061 | 156.8195 | 165.8251 |
| 735.1666 | 1769.698 | 431.1096 | 357.1381 | 160.2336 | 187.224 |
| 501.2355 | 161.0151 | 475.7576 | 355.912 | 168.2427 | 207.4631 |
| 1992.777 | 429.2316 | 527.6454 | 414.35 | 171.4597 | 164.5472 |
| 421.135 | 414.3858 | 559.1635 | 419.3163 | 152.9504 | 158.8847 |
| 609.1648 | 577.1931 | 484.2299 | 154.3275 | 157.5492 | 204.3626 |
| 409.0599 | 1566.543 | 1212.176 | 693.2978 | 338.2353 | 220.6954 |
| 472.2283 | 1559.401 | 614.2717 | 163.7952 | 171.4033 | 162.0643 |
| 414.384 | 157.0201 | 581.13 | 332.5316 | 186.8339 | 172.1728 |
| 458.1779 | 473.2544 | 322.1008 | 681.7949 | 163.3473 | 155.0985 |
| 413.0852 | 532.569 | 744.3627 | 591.2671 | 165.7355 | 165.238 |
| 327.0622 | 184.2636 | 310.8001 | 156.8889 | 154.0182 | 255.9389 |
| 641.1511 | 466.3958 | 1063.472 | 269.0567 | 160.1992 | 168.2581 |
| 462.1488 | 1658.54 | 651.5467 | 256.0981 | 165.4599 | 159.1852 |
| 1990.742 | 435.1145 | 161.5183 | 529.5776 | 337.3125 | 161.1694 |
| 364.1765 | 707.3803 | 622.0993 | 168.0339 | 162.9292 | 157.1942 |
| 182.9982 | 1657.36 | 178.0487 | 508.3069 | 160.7617 | 251.0581 |
| 456.9795 | 376.2493 | 1111.953 | 398.8308 | 193.8122 | 190.7965 |
| 309.1245 | 207.1991 | 152.0598 | 160.8388 | 222.7713 | 155.7722 |
| 521.1777 | 1029.474 | 483.2097 | 641.0958 | 334.07 | 237.3296 |
| 294.0618 | 792.1265 | 386.2117 | 165.1366 | 161.8343 | 219.1779 |
| 435.2944 | 471.2058 | 155.9266 | 184.2477 | 149.1194 | 164.2035 |
| 441.1767 | 437.2372 | 446.4427 | 367.7022 | 297.1145 | 150.0782 |
| 382.4892 | 184.6088 | 811.2278 | 390.0832 | 231.1508 | 200.2322 |
| 382.0907 | 1467.051 | 508.3433 | 160.576 | 148.5656 | 161.7098 |
| 442.2176 | 470.0419 | 495.1729 | 204.3543 | 162.536 | 168.0781 |
| 341.1143 | 613.1962 | 608.2811 | 152.6914 | 316.4638 | 205.3843 |
| 604.2583 | 637.161 | 548.1483 | 651.2368 | 209.2748 | 170.6283 |
| 465.0939 | 493.1941 | 168.6944 | 824.1139 | 164.5889 | 163.6979 |
| 471.0699 | 422.1245 | 561.2434 | 619.2531 | 250.2334 | 164.4359 |
| 255.1602 | 535.1564 | 546.1338 | 253.1881 | 167.097 | 159.4568 |
| 461.2182 | 471.1967 | 391.3052 | 163.6924 | 223.466 | 159.9146 |
| 439.1703 | 1020.017 | 920.5404 | 162.0433 | 275.8898 | 166.6897 |
| 544.2447 | 424.1692 | 174.3256 | 167.8395 | 166.5734 | 164.1112 |
| 419.1195 | 191.9743 | 156.8612 | 162.741 | 162.8985 | 212.5598 |
| 355.1862 | 310.8066 | 524.4981 | 590.0853 | 157.7904 | 156.354 |
| 345.0727 | 501.1837 | 620.4983 | 620.5011 | 301.0089 | 193.6984 |
| 278.8842 | 529.3274 | 375.1172 | 161.3209 | 162.9853 | 151.1903 |
| 257.0543 | 459.2 | 469.1449 | 226.0283 | 153.1225 | 165.9765 |
| 427.1358 | 1772.103 | 616.5555 | 796.0541 | 283.2983 | 165.6248 |
| 310.7809 | 310.5347 | 362.1641 | 476.4317 | 165.0989 | 203.2517 |
| 526.2765 | 1077.461 | 342.1302 | 177.539 | 177.5966 | 208.3586 |
| 331.518 | 1243.057 | 656.2252 | 713.9202 | 205.9834 | 223.5199 |
| 406.2597 | 276.2409 | 1415.849 | 154.4656 | 196.0725 | 188.2888 |
| 482.1932 | 575.9043 | 378.0508 | 495.7928 | 318.6141 | 212.8519 |
| 241.5254 | 466.0655 | 839.3496 | 349.1043 | 202.0494 | 161.0535 |
| 411.0561 | 521.1072 | 550.1719 | 360.0724 | 302.9111 | 238.4466 |
| 503.2146 | 310.8069 | 567.9513 | 159.1246 | 161.6104 | 178.2628 |
| 513.1431 | 478.5113 | 162.7914 | 639.9952 | 161.7735 | 167.4201 |
| 461.1815 | 423.258 | 724.4252 | 414.3652 | 197.0462 | 151.1434 |
| 382.5109 | 236.7742 | 552.482 | 296.3883 | 161.2005 | 201.2955 |
| 181.0336 | 528.3204 | 482.2432 | 332.5938 | 157.0332 | 160.3588 |
| 603.1426 | 158.6682 | 1227.163 | 700.9543 | 317.4234 | 167.0345 |
| 460.146 | 552.5003 | 513.5357 | 399.2612 | 300.3405 | 153.9345 |
| 556.1826 | 208.0074 | 378.0495 | 590.4782 | 154.2978 | 150.5289 |
| 480.2602 | 368.1901 | 1155.753 | 162.8552 | 148.3328 | 156.5277 |
| 337.1503 | 600.3896 | 486.7563 | 298.4088 | 227.3709 | 220.4944 |
| 391.067 | 261.6981 | 503.2471 | 164.9191 | 148.0492 | 199.0734 |
| 446.1173 | 478.1139 | 997.3025 | 654.7872 | 164.2109 | 166.5249 |
| 528.3211 | 710.3881 | 801.4536 | 371.1611 | 342.4622 | 161.4453 |
| 207.1944 | 495.1447 | 517.2417 | 176.9708 | 161.2121 | 176.4801 |
| 333.1919 | 700.3521 | 166.3112 | 633.6352 | 170.0526 | 206.7191 |
| 243.0662 | 1437.774 | 711.5736 | 506.8314 | 175.1411 | 170.5573 |
| 464.0987 | 1961.312 | 952.5113 | 299.3116 | 272.1183 | 162.6775 |
| 358.0892 | 489.1989 | 474.1778 | 505.4524 | 151.8605 | 194.7065 |
| 454.1617 | 1397.298 | 1344.063 | 654.1005 | 156.4191 | 216.0438 |
| 381.0704 | 161.3315 | 594.6748 | 445.775 | 206.0072 | 201.5394 |
| 413.057 | 535.1255 | 518.366 | 603.7753 | 165.1442 | 191.8617 |
| 197.082 | 372.0837 | 161.7737 | 679.1253 | 163.8191 | 159.3748 |
| 160.3594 | 480.1658 | 1076.6 | 686.1944 | 219.2046 | 167.8441 |
| 226.0265 | 458.186 | 996.8727 | 320.1707 | 312.5981 | 183.7503 |
| 209.9867 | 1004.559 | 305.1098 | 695.1092 | 163.5825 | 147.4493 |
| 411.0687 | 552.514 | 453.2243 | 677.1325 | 216.9736 | 165.7513 |
| 248.6365 | 546.2463 | 449.2734 | 358.2719 | 317.0514 | 165.4708 |
| 159.5546 | 710.2837 | 265.083 | 413.1416 | 161.7684 | 156.0955 |
| 534.1826 | 482.1094 | 1279.235 | 406.1999 | 250.101 | 233.9791 |
| 342.083 | 613.3746 | 699.2115 | 161.3255 | 163.9335 | 199.2955 |
| 713.1721 | 484.2779 | 994.4143 | 502.4965 | 164.5847 | 175.0482 |
| 264.0513 | 621.1308 | 154.0466 | 690.3716 | 269.9006 | 229.754 |
| 527.2796 | 563.3356 | 434.1535 | 415.163 | 170.8433 | 203.2572 |
| 331.5229 | 346.0391 | 421.0932 | 792.473 | 243.8939 | 208.7267 |
| 160.5901 | 583.6142 | 620.7034 | 164.8657 | 164.7928 | 163.6306 |
| 395.0861 | 1769.324 | 916.8894 | 164.0747 | 148.1397 | 187.873 |
| 564.1545 | 292.515 | 399.2728 | 174.6012 | 153.3205 | 157.1611 |
| 553.9224 | 494.2338 | 581.3366 | 681.0365 | 212.3151 | 153.0362 |
| 355.1682 | 937.3179 | 622.6986 | 425.116 | 165.7345 | 200.7757 |
| 193.6451 | 332.0858 | 469.9547 | 206.2225 | 169.374 | 159.9521 |
| 452.0546 | 512.1199 | 165.9999 | 160.0324 | 162.9171 | 174.2491 |
| 585.2317 | 162.6843 | 382.4392 | 367.516 | 337.018 | 157.6775 |
| 292.4996 | 322.1043 | 596.3053 | 399.1246 | 154.773 | 166.7497 |
| 614.1337 | 1901.641 | 526.3377 | 238.3893 | 177.5992 | 185.1767 |
| 325.104 | 508.1251 | 785.1645 | 169.3169 | 324.282 | 153.7136 |
| 407.1863 | 1119.181 | 763.7109 | 151.9424 | 196.0846 | 165.9742 |
| 276.2595 | 415.2019 | 156.616 | 158.7805 | 159.6473 | 157.6605 |
| 496.9782 | 1022.104 | 654.7464 | 205.8116 | 253.9608 | 187.8054 |
| 453.0862 | 710.376 | 1158.66 | 164.5346 | 255.5023 | 171.4777 |
| 160.0949 | 1768.8 | 631.2339 | 374.5998 | 166.6302 | 177.9232 |
| 357.0973 | 621.959 | 162.108 | 677.5872 | 173.2962 | 158.6166 |
| 223.0976 | 1286.025 | 848.4819 | 166.1825 | 250.5271 | 165.7627 |
| 569.1303 | 428.1835 | 339.1238 | 417.0937 | 208.2665 | 165.9647 |
| 161.8169 | 460.1072 | 164.1838 | 400.1186 | 284.786 | 210.1613 |
| 370.1225 | 357.0727 | 203.922 | 179.8032 | 278.8804 | 170.3143 |
| 427.0549 | 401.1276 | 159.2266 | 147.9756 | 164.0276 | 150.8942 |
| 937.3163 | 450.1453 | 381.0992 | 164.7195 | 167.5739 | 205.2154 |
| 452.0593 | 1713.646 | 653.5059 | 156.8719 | 275.0196 | 158.064 |
| 194.0508 | 535.9117 | 153.4412 | 718.2271 | 159.2376 | 182.3318 |
| 192.0666 | 411.0749 | 746.3562 | 166.6655 | 340.1152 | 161.9646 |
| 267.004 | 251.1118 | 184.1672 | 497.1205 | 335.0985 | 157.5752 |
| 508.0886 | 388.1131 | 1083.529 | 632.4231 | 159.6406 | 192.3053 |
| 612.1836 | 413.0989 | 565.193 | 320.2341 | 254.266 | 153.4688 |
| 1990.644 | 1455.735 | 492.1753 | 348.8074 | 158.7206 | |
| 740.3299 | 1099.317 | 863.2299 | 254.5873 | 162.8661 | |
| 453.1109 | 592.3263 | 1368.283 | 725.5014 | 154.3046 | |
| 536.2267 | 292.4887 | 450.7891 | 159.5617 | 164.3721 | |
| 473.2617 | 159.8105 | 377.8503 | 154.8055 | 160.902 | |
| 420.8249 | 443.213 | 522.2568 | 679.3038 | 269.6262 | |
| 571.2563 | 161.2257 | 448.441 | 167.7627 | 158.9664 | |

TABLE 3
List of biomarker determinants that correlate with ICD (each number
is the m/z ratio of a specific biomarker determinant).

| | | | | | |
|---|---|---|---|---|---|
| 521.331 | 1321.068 | 625.5521 | 467.3288 | 292.4862 | 180.0528 |
| 520.328 | 607.1955 | 1992.03 | 297.2499 | 153.1561 | 337.3893 |
| 356.1827 | 834.2559 | 617.1875 | 299.2502 | 151.9479 | 343.2856 |
| 508.328 | 472.1743 | 480.2789 | 297.2372 | 161.2011 | 161.9232 |
| 540.3363 | 458.318 | 216.2113 | 299.2724 | 272.5671 | 194.7471 |
| 535.3389 | 541.9298 | 198.9082 | 368.25 | 204.7189 | 178.1162 |
| 536.3229 | 715.545 | 667.1673 | 221.1013 | 248.4571 | 159.5477 |
| 484.2374 | 421.1244 | 542.0323 | 807.5667 | 222.0143 | 367.8781 |
| 451.216 | 388.2435 | 1326.463 | 297.2311 | 214.5182 | 163.2811 |
| 552.3 | 280.0781 | 662.4204 | 457.1939 | 165.9834 | 148.5945 |
| 595.3248 | 593.1557 | 383.1247 | 287.9732 | 211.0724 | 150.578 |
| 492.3331 | 453.6611 | 573.1979 | 160.7231 | 228.5196 | 250.8875 |
| 399.2211 | 159.5615 | 1784.457 | 280.1191 | 203.9816 | 315.0754 |
| 522.3071 | 1243.072 | 510.1053 | 297.2476 | 168.4986 | 168.9149 |
| 397.2054 | 1172.257 | 528.0388 | 396.1412 | 206.8067 | 158.5764 |
| 522.2371 | 421.3673 | 1011.538 | 843.6358 | 153.87 | 171.2599 |
| 464.1827 | 640.3176 | 310.7981 | 392.2924 | 320.9233 | 250.2938 |
| 558.3276 | 439.1513 | 1264.301 | 226.0106 | 168.4527 | 323.644 |
| 596.3283 | 496.9733 | 659.1975 | 392.2669 | 189.3964 | 226.0226 |
| 544.2586 | 637.3096 | 391.2986 | 207.0857 | 154.8261 | 153.8438 |
| 368.1618 | 483.7502 | 210.3257 | 365.2308 | 190.6813 | 148.2227 |
| 505.3285 | 604.3255 | 1263.695 | 301.2635 | 148.6153 | 160.8807 |
| 395.1897 | 520.8958 | 597.1801 | 510.2894 | 300.0802 | 285.2565 |
| 448.3069 | 382.5012 | 1654.578 | 163.1378 | 257.0779 | 194.0925 |
| 525.2341 | 645.2068 | 843.5895 | 492.1836 | 157.7147 | 174.2493 |
| 519.344 | 566.1362 | 489.2535 | 160.4676 | 181.9867 | 230.6438 |
| 452.2194 | 433.377 | 656.0574 | 168.9844 | 149.1422 | 159.7837 |
| 521.2337 | 948.9276 | 159.5265 | 556.3554 | 155.9676 | 171.6084 |
| 367.1584 | 382.5143 | 946.1074 | 613.3007 | 217.3534 | 158.993 |
| 409.287 | 937.6762 | 409.262 | 496.2738 | 168.3674 | 152.8811 |
| 556.3313 | 1126.987 | 719.2093 | 368.1067 | 243.7672 | 216.1867 |
| 408.2871 | 182.5605 | 682.2986 | 525.2238 | 231.5747 | 224.5548 |
| 408.2958 | 164.3579 | 707.5482 | 310.5337 | 312.1542 | 188.2999 |
| 408.2836 | 452.6539 | 774.2417 | 207.0492 | 157.5811 | 168.2837 |
| 804.5473 | 1124.808 | 1526.049 | 165.7492 | 236.7711 | 198.8959 |
| 409.2906 | 468.1626 | 1534.543 | 260.0234 | 217.7402 | 196.4474 |
| 498.2977 | 511.3088 | 1482.947 | 369.0956 | 205.681 | 165.528 |
| 171.4749 | 490.1946 | 445.2492 | 310.7879 | 162.7421 | 160.2933 |
| 478.3174 | 989.1504 | 923.8975 | 299.248 | 295.7594 | 157.084 |
| 359.1573 | 163.4508 | 272.1289 | 331.486 | 287.1248 | 253.699 |
| 407.2803 | 714.1167 | 573.2953 | 610.1273 | 155.8348 | 162.6669 |
| 398.2088 | 252.146 | 216.2067 | 556.3642 | 287.5284 | 147.8277 |
| 557.3347 | 490.2361 | 389.1118 | 392.2981 | 157.6577 | 155.6476 |
| 538.3207 | 802.3483 | 466.5722 | 564.3653 | 154.3 | 162.0615 |
| 220.0979 | 678.7307 | 1114.692 | 329.1354 | 282.8445 | 305.3487 |
| 407.2683 | 258.9866 | 883.2303 | 235.1169 | 168.1579 | 163.9512 |
| 410.2904 | 548.2805 | 155.3972 | 160.0565 | 156.1688 | 197.4904 |
| 409.2844 | 207.5951 | 743.262 | 443.1784 | 222.7749 | 151.4511 |
| 515.2878 | 620.295 | 441.1611 | 541.2476 | 166.2155 | 154.1652 |
| 164.0717 | 732.5928 | 654.3042 | 471.0991 | 171.4484 | 158.8404 |
| 160.8377 | 419.6486 | 518.1895 | 729.1648 | 193.3878 | 171.1914 |
| 407.3044 | 490.2728 | 552.5062 | 181.9894 | 201.327 | 162.8735 |
| 259.1299 | 431.4262 | 759.4228 | 383.0805 | 169.8308 | 236.772 |
| 407.2564 | 179.8886 | 174.3269 | 275.1687 | 210.0102 | 164.4203 |
| 203.0326 | 681.5826 | 1060.779 | 265.0565 | 165.4516 | 165.1281 |
| 410.2879 | 1108.337 | 158.8177 | 553.2287 | 225.4904 | 318.4277 |
| 514.2844 | 478.6367 | 443.27 | 183.9876 | 181.1466 | 161.4585 |
| 521.3232 | 333.1525 | 592.3482 | 306.1744 | 160.3951 | 248.6052 |
| 462.3227 | 164.982 | 1133.097 | 627.3022 | 232.2478 | 168.4217 |
| 161.3073 | 161.7094 | 569.5099 | 407.2954 | 156.1845 | 182.3713 |
| 276.117 | 427.9922 | 499.2073 | 690.1756 | 154.8821 | 224.6313 |
| 467.2108 | 156.5305 | 161.6428 | 709.1899 | 155.5002 | 168.9842 |
| 407.2924 | 409.4682 | 155.2113 | 312.0912 | 153.4645 | 168.7408 |
| 161.3111 | 237.7979 | 903.4416 | 593.1508 | 171.1465 | 152.2196 |
| 289.1558 | 643.7773 | 497.1929 | 569.2939 | 163.3631 | 197.4554 |
| 506.3123 | 655.7941 | 160.4132 | 518.3123 | 166.7612 | 168.9991 |
| 161.3092 | 522.0419 | 461.1023 | 552.1439 | 147.9506 | 204.0501 |
| 400.2244 | 695.2317 | 266.7878 | 276.2385 | 154.0329 | 166.158 |
| 408.2716 | 450.865 | 638.6652 | 589.3352 | 189.1571 | 160.3839 |
| 425.2002 | 746.1 | 350.4937 | 1992.979 | 149.0383 | 316.1251 |
| 245.1143 | 682.2497 | 445.1402 | 710.2969 | 148.0176 | 158.7751 |
| 279.0275 | 153.5691 | 633.3012 | 727.4799 | 167.4742 | 301.0114 |
| 362.1457 | 320.0178 | 468.3809 | 248.0962 | 154.3766 | 166.2372 |
| 218.1034 | 182.071 | 412.1921 | 181.9872 | 160.888 | 230.3316 |
| 570.3437 | 212.8538 | 403.2166 | 516.1442 | 151.8313 | 201.7695 |
| 180.0667 | 595.3118 | 710.6406 | 173.0453 | 261.7052 | 169.5677 |
| 477.2744 | 603.645 | 895.7251 | 552.4555 | 529.1199 | 237.1967 |
| 587.2875 | 590.2562 | 737.1561 | 261.6968 | 463.2233 | 169.626 |
| 367.13 | 424.4183 | 210.8008 | 709.1866 | 457.0905 | 199.291 |
| 301.0095 | 682.5555 | 403.4244 | 800.5288 | 253.1081 | 191.7645 |
| 487.2371 | 672.9652 | 909.2483 | 292.4801 | 457.1538 | 164.4238 |
| 294.1347 | 261.6993 | 208.8251 | 407.0724 | 781.5624 | 161.8092 |
| 454.2349 | 429.2454 | 165.7567 | 414.3554 | 206.0459 | 158.8228 |
| 490.3172 | 216.4641 | 381.745 | 536.3425 | 356.1463 | 187.3308 |
| 467.2472 | 685.2374 | 241.5259 | 610.3077 | 169.5462 | 269.0389 |
| 276.2368 | 150.6897 | 452.0476 | 465.3122 | 467.3 | 149.0406 |
| 465.2389 | 651.968 | 597.3733 | 292.4849 | 601.3052 | 201.6528 |
| 181.0506 | 319.9856 | 463.5382 | 369.1075 | 727.3759 | 162.0681 |
| 227.1401 | 662.6736 | 412.6537 | 414.1123 | 820.5424 | 161.3487 |
| 611.3201 | 388.8026 | 493.3371 | 226.0121 | 836.5377 | 148.6354 |
| 476.271 | 197.6176 | 198.7786 | 292.4844 | 786.5884 | 165.2959 |
| 430.1943 | 150.2271 | 422.6076 | 527.1044 | 258.1459 | 163.118 |
| 453.2316 | 301.4129 | 315.0542 | 292.4768 | 807.4384 | 205.7313 |
| 526.2374 | 212.5376 | 441.2347 | 800.4821 | 226.0162 | 162.149 |
| 368.1333 | 171.6452 | 634.0898 | 1509.47 | 470.2297 | 165.7372 |
| 475.2676 | 299.2558 | 213.9282 | 177.58 | 474.2553 | 165.5898 |
| 328.1191 | 355.7744 | 643.689 | 498.133 | 443.2428 | 167.2011 |
| 261.0075 | 168.7711 | 638.4457 | 957.4957 | 499.2371 | 171.9309 |
| 181.0483 | 159.6238 | 219.5659 | 401.0912 | 398.3275 | 161.7488 |
| 231.135 | 276.7675 | 384.9531 | 368.6954 | 796.5346 | 247.9245 |
| 275.1136 | 385.1206 | 430.1517 | 486.1332 | 161.9054 | 164.5638 |
| 509.219 | 390.9344 | 686.2505 | 1995.079 | 467.3112 | 170.7376 |
| 341.1467 | 337.1517 | 359.2734 | 656.0566 | 633.0865 | 166.5493 |
| 182.054 | 163.7402 | 151.8925 | 292.4794 | 302.1722 | 154.6827 |
| 564.3026 | 161.9669 | 421.8623 | 730.1684 | 542.3197 | 200.179 |
| 352.1765 | 361.0961 | 279.0164 | 295.138 | 241.527 | 158.6252 |
| 351.135 | 365.904 | 173.4654 | 633.302 | 621.645 | 150.678 |
| 543.2552 | 219.0508 | 163.0984 | 310.7626 | 165.0419 | 205.6162 |
| 466.2422 | 229.3685 | 155.9087 | 554.5932 | 300.0547 | 160.4744 |
| 358.0796 | 211.6726 | 416.2663 | 494.3399 | 709.2621 | 159.8309 |
| 488.2404 | 211.6322 | 164.4698 | 355.0858 | 487.2466 | 188.0173 |
| 393.0649 | 165.2223 | 166.4976 | 400.2089 | 410.3276 | 165.3859 |
| 365.0726 | 267.4561 | 204.0667 | 596.5042 | 620.3935 | 190.908 |
| 280.0309 | 338.5023 | 374.0234 | 869.2145 | 159.4819 | 159.8902 |
| 491.1469 | 369.1374 | 168.4285 | 813.4581 | 509.2578 | 578.3465 |
| 403.249 | 307.706 | 266.5246 | 452.0194 | 812.5399 | 190.0545 |
| 526.2845 | 166.7798 | 184.2515 | 367.0844 | 251.0303 | 393.2957 |
| 661.178 | 225.3889 | 382.5202 | 677.3023 | 292.5057 | 160.5481 |
| 217.0273 | 155.193 | 262.8851 | 847.6508 | 370.0002 | 485.2578 |
| 291.1086 | 310.7988 | 397.0174 | 622.3848 | 216.1875 | 239.1289 |
| 528.2426 | 226.0251 | 348.723 | 487.2174 | 349.2418 | 455.2146 |
| 369.1741 | 289.1115 | 376.1219 | 355.1595 | 187.1169 | 489.2715 |
| 486.2528 | 263.2056 | 165.7594 | 533.3233 | 467.1307 | 544.1459 |
| 623.1618 | 162.6829 | 154.7738 | 163.9651 | 216.1981 | 783.5331 |
| 612.3235 | 250.8377 | 209.9549 | 980.5662 | 434.1304 | 445.194 |
| 295.2279 | 158.2688 | 373.9043 | 276.2342 | 328.0861 | 329.2333 |
| 439.2975 | 193.6663 | 159.7999 | 1589.634 | 1990.849 | 169.5441 |
| 357.0762 | 227.1297 | 367.1104 | 514.2651 | 429.0497 | 936.53 |
| 181.0456 | 218.7757 | 155.4164 | 416.2848 | 545.129 | 824.5658 |
| 455.2384 | 209.1031 | 341.0095 | 505.2213 | 1992.992 | 579.3498 |
| 527.2392 | 164.2345 | 216.9838 | 581.6334 | 469.2334 | 163.2999 |
| 203.0826 | 190.0349 | 170.0571 | 310.7618 | 487.2418 | 465.2886 |
| 613.3272 | 160.7765 | 171.4676 | 461.0548 | 1990.891 | 504.2449 |
| 466.3254 | 207.1924 | 186.7898 | 1988.634 | 504.1777 | 452.0504 |
| 721.1985 | 194.0821 | 236.787 | 1907.204 | 399.0392 | 355.1534 |
| 429.2971 | 151.3667 | 247.7403 | 1396.018 | 527.2515 | 545.279 |
| 509.3315 | 218.8535 | 161.0201 | 161.5827 | 592.1389 | 315.2541 |
| 456.2307 | 334.4109 | 166.6306 | 276.236 | 349.1194 | 520.1653 |
| 163.2462 | 172.874 | 194.217 | 861.8754 | 310.7734 | 276.2533 |
| 594.3412 | 332.2647 | 294.9415 | 398.8574 | 184.2492 | 481.1351 |
| 455.2275 | 164.4154 | 152.5557 | 707.165 | 530.1294 | 500.3133 |
| 571.3467 | 289.8723 | 159.7008 | 567.1988 | 786.5894 | 326.1721 |
| 381.1456 | 244.8865 | 165.0194 | 462.6873 | 382.4781 | 198.0772 |
| 426.1266 | 165.7274 | 197.6006 | 571.2885 | 383.0445 | 541.3204 |
| 447.0867 | 158.4244 | 165.5294 | 164.5844 | 472.226 | 593.3257 |
| 255.2329 | 187.652 | 301.4396 | 426.1124 | 616.9668 | 505.2386 |
| 385.0904 | 265.4522 | 167.3136 | 671.8397 | 456.2452 | 957.5093 |
| 415.2814 | 188.0567 | 189.7006 | 619.1669 | 411.1329 | 343.067 |
| 497.2942 | 165.0854 | 285.5782 | 1918.558 | 879.2985 | 339.0933 |
| 329.122 | 161.9248 | 334.1644 | 1643.324 | 455.2624 | 472.262 |
| 453.2022 | 239.105 | 338.669 | 446.7391 | 575.1198 | 159.2821 |
| 354.1055 | 261.6896 | 202.6434 | 1769.483 | 408.2942 | 399.1521 |
| 488.1232 | 222.2607 | 158.4537 | 679.0168 | 475.2138 | 799.5626 |
| 407.2706 | 176.5055 | 272.0215 | 355.1136 | 382.4864 | 158.7881 |
| 292.4834 | 158.9093 | 270.2968 | 1957.591 | 578.1381 | 539.3225 |
| 179.035 | 160.022 | 157.9889 | 164.9336 | 570.136 | |
| 620.3204 | 166.1537 | 167.5211 | 156.1778 | 440.1101 | |
| 296.2312 | 158.1676 | 214.5124 | 184.2463 | 662.6459 | |
| 489.2329 | 189.5777 | 165.8579 | 163.113 | 558.3386 | |
| 722.2019 | 318.2314 | 328.7549 | 348.9114 | 460.2374 | |
| 160.8415 | 161.3664 | 162.8726 | 636.1254 | 276.2451 | |
| 438.2942 | 255.9743 | 152.2101 | 1405.729 | 556.3085 | |
| 481.2264 | 153.5234 | 147.5389 | 514.2761 | 517.2263 | |
| 374.1569 | 160.4257 | 160.034 | 163.1491 | 1988.671 | |
| 504.333 | 214.7872 | 156.483 | 165.7917 | 487.2533 | |
| 299.2527 | 162.7017 | 161.3339 | 310.7719 | 251.0786 | |
| 464.3018 | 165.3735 | 156.853 | 710.2875 | 331.4854 | |
| 437.2908 | 237.0284 | 202.2166 | 153.6001 | 880.5326 | |
| 467.236 | 166.1486 | 204.3585 | 185.4187 | 551.2444 | |
| 160.2096 | 158.3015 | 166.5753 | 821.3967 | 236.7725 | |
| 563.299 | 256.0576 | 209.8564 | 520.3469 | 226.0131 | |
| 181.0461 | 254.1761 | 171.4902 | 468.3033 | 385.1769 | |
| 785.5848 | 210.6478 | 155.0812 | 570.172 | 541.2674 | |
| 555.352 | 337.9008 | 161.356 | 593.263 | 506.2678 | |
| 821.5548 | 164.0107 | 223.2302 | 530.1366 | 456.2355 | |
| 705.167 | 207.0835 | 152.2267 | 541.1828 | 475.2446 | |
| 299.2457 | 156.5896 | 192.6455 | 165.5851 | 1956.427 | |
| 465.3221 | 159.3622 | 158.881 | 497.2084 | 1988.708 | |
| 523.31 | 176.7512 | 189.0792 | 645.1153 | 1954.153 | |
| 510.2531 | 174.39 | 263.4622 | 409.7866 | 423.1528 | |
| 386.0938 | 180.4063 | 148.2376 | 1243.644 | 396.1927 | |
| 300.2625 | 185.2402 | 197.7336 | 1506.245 | 470.2582 | |
| 236.7731 | 216.2083 | 245.802 | 1109.805 | 948.7857 | |
| 540.2636 | 225.8743 | 301.5152 | 488.437 | 156.2424 | |
| 554.3487 | 159.1355 | 159.4785 | 163.5505 | 382.4899 | |
| 589.3031 | 307.8729 | 202.7938 | 165.7789 | 455.2782 | |
| 605.3668 | 160.6108 | 228.8444 | 486.8872 | 454.2058 | |
| 163.145 | 283.0392 | 290.6396 | 1206.42 | 487.3435 | |
| 191.0561 | 174.4912 | 262.5522 | 813.5823 | 549.0875 | |
| 499.2046 | 206.0429 | 244.0755 | 846.2881 | 536.3167 | |
| 784.5814 | 263.3978 | 158.2437 | 874.9454 | 473.206 | |
| 299.2591 | 257.4285 | 158.6774 | 476.2786 | 612.288 | |
| 516.2913 | 205.3228 | 290.7598 | 820.5436 | 877.2819 | |
| 534.3071 | 256.7538 | 167.5776 | 389.0852 | 992.5091 | |
| 299.2466 | 149.6841 | 157.7889 | 355.1556 | 815.4708 | |
| 512.2687 | 171.4735 | 179.9013 | 939.5323 | 573.3405 | |
| 783.5781 | 160.574 | 244.3513 | 382.4852 | 590.2992 | |
| 331.484 | 163.6537 | 211.7447 | 1037.521 | 555.2825 | |
| 549.2211 | 166.4413 | 166.1675 | 163.9985 | 530.2842 | |
| 160.7546 | 194.4298 | 166.9649 | 430.9155 | 482.3026 | |
| 405.2528 | 293.8429 | 192.2563 | 353.5896 | 581.3097 | |
| 282.444 | 235.3589 | 173.6528 | 216.9434 | 625.7609 | |
| 653.3389 | 156.9547 | 163.8378 | 183.0549 | 165.7455 | |
| 283.2643 | 194.1276 | 172.899 | 236.7761 | 1037.867 | |
| 506.2605 | 262.4753 | 203.5023 | 532.1543 | 636.2894 | |
| 662.5361 | 204.0948 | 197.2064 | 184.3239 | 470.2662 | |
| 597.3323 | 150.9448 | 216.4763 | 432.431 | 1979.805 | |
| 490.3538 | 163.7873 | 184.1771 | 163.2852 | 1571.216 | |
| 505.2572 | 249.2551 | 172.3668 | 164.9614 | 407.2975 | |
| 331.4919 | 168.439 | 168.2574 | 193.8941 | 226.0086 | |
| 799.5729 | 160.9332 | 177.5945 | 381.1212 | 1020.203 | |
| 465.1069 | 155.394 | 236.7922 | 436.1373 | 586.3093 | |
| 406.268 | 170.2068 | 163.7567 | 555.8502 | 414.098 | |
| 285.2436 | 275.2174 | 172.921 | 635.0369 | 839.3798 | |
| 271.8563 | 147.5059 | 164.2527 | 465.0804 | 487.2598 | |
| 707.1829 | 148.936 | 220.1509 | 1006.211 | 438.8268 | |
| 558.3379 | 186.9812 | 158.0088 | 157.0918 | 481.3022 | |
| 590.3065 | 239.5641 | 177.1354 | 705.3348 | 369.1543 | |
| 539.3243 | 148.9727 | 158.5227 | 642.8995 | 537.2526 | |
| 187.1089 | 165.4058 | 180.4254 | 609.0171 | 864.0379 | |
| 800.5763 | 264.8013 | 222.7227 | 520.5772 | 639.4949 | |
| 302.1357 | 164.4535 | 174.775 | 500.2322 | 552.4631 | |
| 405.2646 | 161.6878 | 199.1395 | 434.1037 | 453.2452 | |
| 292.4895 | 165.8969 | 164.2279 | 161.7777 | 556.2585 | |
| 506.3487 | 151.7926 | 163.2243 | 343.1515 | 637.288 | |
| 215.1401 | 161.7227 | 204.2152 | 231.0878 | 633.3754 | |
| 822.5582 | 148.64 | 175.6529 | 1328.985 | 475.261 | |
| 577.2969 | 152.7334 | 155.3894 | 455.1405 | 592.1992 | |
| 507.2639 | 156.26 | 148.7578 | 687.5024 | 541.9024 | |
| 801.5799 | 156.8449 | 151.0835 | 683.1165 | 892.6652 | |
| 248.6201 | 166.4691 | 158.9455 | 497.2189 | 967.5523 | |
| 392.2999 | 163.4147 | 217.1194 | 167.2762 | 276.239 | |
| 373.1729 | 192.4785 | 585.2718 | 892.0059 | 261.6952 | |
| 359.0721 | 157.9286 | 342.265 | 1097.18 | 948.326 | |
| 799.4806 | 158.1144 | 476.3016 | 446.778 | 772.5378 | |
| 591.345 | 164.1638 | 375.1847 | 484.0704 | 533.4067 | |
| 406.2446 | 177.1949 | 162.2701 | 639.9982 | 1198.993 | |
| 226.0327 | 213.6257 | 163.8775 | 492.1592 | 401.2654 | |
| 626.2194 | 185.4225 | 414.3602 | 207.1803 | 1230.175 | |
| 206.0823 | 167.3571 | 490.1435 | 322.7797 | 469.2428 | |
| 708.1862 | 279.233 | 408.3078 | 579.836 | 710.3044 | |
| 505.2195 | 301.2659 | 465.2735 | 840.5621 | 544.5294 | |
| 411.1846 | 537.3255 | 162.0122 | 564.1449 | 1182.822 | |
| 321.2435 | 365.0185 | 466.3077 | 404.2524 | 570.2146 | |
| 316.1514 | 430.2268 | 161.941 | 531.4612 | 483.7793 | |
| 391.2999 | 590.3415 | 405.2885 | 304.6027 | 603.2424 | |
| 469.2106 | 446.2573 | 162.2682 | 898.9717 | 199.9243 | |
| 647.1981 | 817.5747 | 244.1303 | 817.4775 | 710.2691 | |
| 527.2878 | 559.3551 | 714.537 | 551.0947 | 1109.808 | |
| 802.5833 | 816.5713 | 281.1394 | 995.4609 | 338.2417 | |
| 276.2352 | 845.615 | 393.2921 | 549.7227 | 1427.544 | |
| 219.1068 | 261.017 | 165.0323 | 639.284 | 614.3156 | |
| 207.1812 | 527.1994 | 538.3538 | 163.6086 | 159.1518 | |
| 207.1794 | 476.2781 | 161.7863 | 367.0937 | 460.2796 | |
| 243.0445 | 443.2569 | 482.1385 | 423.0756 | 171.4592 | |
| 453.2411 | 635.4279 | 504.1591 | 526.5486 | 865.197 | |
| 158.8086 | 815.5679 | 480.1228 | 236.7689 | 602.8487 | |
| 449.2005 | 156.8491 | 392.2887 | 169.3139 | 671.7468 | |
| 322.2469 | 165.7416 | 465.3044 | 522.0254 | 870.174 | |
| 501.2568 | 337.2384 | 277.1194 | 736.4317 | 1183.455 | |
| 276.2405 | 810.5502 | 813.5523 | 455.8397 | 894.1025 | |
| 281.2486 | 800.484 | 557.3484 | 353.0706 | 149.2475 | |
| 161.3067 | 513.1097 | 225.1132 | 884.5536 | 870.122 | |
| 248.6108 | 262.0203 | 561.3409 | 702.3674 | 383.1612 | |
| 408.2599 | 213.0227 | 549.3544 | 579.1349 | 484.2011 | |
| 303.2329 | 688.2835 | 814.5556 | 624.0724 | 307.5395 | |
| 408.1161 | 1990.688 | 391.2853 | 162.634 | 388.1919 | |
| 799.5265 | 172.0979 | 165.7438 | 483.902 | 560.616 | |
| 1990.811 | 715.2838 | 507.3521 | 707.1113 | 514.2432 | |
| 292.4875 | 282.9989 | 815.4718 | 406.28 | 166.4373 | |
| 393.1225 | 486.2611 | 248.6072 | 719.5909 | 633.3649 | |
| 485.2214 | 282.252 | 391.2965 | 823.5648 | 524.2323 | |
| 522.3263 | 595.3445 | 523.2371 | 556.1779 | 671.2587 | |
| 437.2772 | 355.1581 | 558.3518 | 1060.761 | 514.0964 | |
| 299.2658 | 161.7847 | 513.2721 | 528.2678 | 444.2065 | |
| 554.3157 | 625.216 | 508.2647 | 886.036 | 621.5218 | |
| 739.5555 | 515.1407 | 588.2911 | 414.3483 | 932.305 | |
| 504.3694 | 297.2435 | 405.2767 | 394.0683 | 162.5567 | |
| 1027.533 | 298.2469 | 411.0755 | 177.5821 | 555.6157 | |
| 798.5608 | 488.1999 | 332.1252 | 708.148 | 865.3948 | |
| 407.2718 | 173.0456 | 355.1625 | 586.1569 | 218.0444 | |
| 388.1725 | 314.2418 | 507.2131 | 261.6903 | 728.7917 | |
| 408.3088 | 489.1402 | 713.534 | 608.4151 | 165.2617 | |
| 554.2795 | 453.2166 | 439.1069 | 559.1814 | 521.2578 | |
| 277.2173 | 390.1307 | 182.9951 | 217.2081 | 530.2792 | |
| 481.2992 | 471.2224 | 392.2776 | 455.2656 | 598.1522 | |
| 797.5574 | 687.2801 | 609.3046 | 382.469 | 281.2388 | |
| 689.1723 | 445.2638 | 406.2561 | 184.1596 | 171.7053 | |
| 463.3065 | 477.3043 | 310.7644 | 153.8765 | 433.1545 | |
| 360.1776 | 501.2526 | 1990.761 | 522.3284 | 314.1723 | |
| 505.3726 | 272.0598 | 835.5341 | 611.3407 | 444.2563 | |
| 458.1853 | 394.2954 | 391.2633 | 628.6485 | 203.8894 | |
| 444.2604 | 288.0547 | 200.1292 | 164.1477 | 457.1517 | |
| 591.3099 | 483.2422 | 236.7811 | 162.7705 | 1031.616 | |
| 405.2413 | 816.5224 | 407.2324 | 617.6911 | 471.194 | |
| 149.1456 | 301.1517 | 611.285 | 487.0117 | 501.1414 | |
| 507.2613 | 207.1824 | 409.2632 | 158.2185 | 511.2564 | |
| 193.0507 | 382.48 | 879.5301 | 555.1724 | 474.397 | |
| 469.2265 | 427.1655 | 606.3704 | 159.1102 | 494.2585 | |
| 419.6533 | 207.1776 | 367.2466 | 528.0516 | 882.9308 | |
| 162.2663 | 816.4752 | 226.0139 | 158.083 | 715.8881 | |
| 526.248 | 838.5532 | 805.5599 | 543.633 | 163.8993 | |
| 857.6149 | 293.2122 | 737.5758 | 379.6971 | 642.2159 | |
| 182.0459 | 261.7025 | 819.5395 | 439.8829 | 445.2092 | |
| 469.1945 | 309.1092 | 253.1193 | 415.1148 | 994.3913 | |
| 516.2802 | 815.5193 | 169.542 | 164.775 | 1082.79 | |
| 248.614 | 837.5498 | 229.1558 | 159.4114 | 986.5407 | |
| 589.3383 | 1995.158 | 304.0227 | 210.7731 | 948.973 | |
| 459.2097 | 452.028 | 491.2084 | 215.8174 | 166.8473 | |
| 445.254 | 605.1511 | 503.1558 | 707.1458 | 636.2386 | |
| 552.3331 | 162.52 | 465.2316 | 167.5217 | 357.1237 | |
| 573.3434 | 568.295 | 711.2775 | 395.0595 | 357.058 | |
| 437.2632 | 547.3253 | 537.1314 | 384.2834 | 557.3038 | |
| 724.5545 | 823.5618 | 314.1357 | 163.7288 | 152.4918 | |
| 399.1925 | 818.5782 | 503.1823 | 405.9393 | 375.2863 | |
| 416.1908 | 393.2895 | 806.5633 | 375.0755 | 658.3053 | |
| 1990.584 | 391.3078 | 473.2252 | 384.0839 | 674.8943 | |
| 1994.898 | 572.3592 | 204.07 | 163.3658 | 872.5189 | |
| 1992.725 | 369.2128 | 414.3549 | 230.0567 | 729.3675 | |
| 326.0047 | 839.5565 | 201.1245 | 409.0717 | 544.1716 | |
| 724.5526 | 574.3468 | 261.6947 | 313.1193 | 155.3881 | |
| 258.9919 | 329.0911 | 532.1178 | 404.1838 | 818.5761 | |
| 1990.684 | 587.1255 | 539.3568 | 207.1785 | 453.2469 | |
| 413.1719 | 578.3002 | 710.3138 | 337.5606 | 832.7677 | |
| 487.2021 | 371.1572 | 452.2785 | 164.0965 | 306.6315 | |
| 1988.472 | 535.137 | 833.5185 | 162.369 | 601.8792 | |
| 469.2203 | 234.1135 | 468.2504 | 151.9447 | 775.3284 | |
| 400.1958 | 800.5298 | 520.3643 | 312.2544 | 468.5083 | |
| 215.1653 | 486.2247 | 498.2895 | 332.1548 | 584.5282 | |
| 310.7859 | 536.3052 | 310.7941 | 166.1673 | 366.4217 | |
| 381.0957 | 271.2278 | 516.1229 | 365.3903 | 163.1119 | |
| 547.2772 | 607.1671 | 346.0558 | 174.6087 | 432.6244 | |
| 423.9815 | 168.4192 | 195.0663 | 167.6237 | 153.9395 | |
| 479.0725 | 328.2493 | 226.0064 | 165.7373 | 236.7745 | |
| 445.9635 | 346.1619 | 484.2455 | 161.4171 | 175.0976 | |
| 545.2617 | 284.2676 | 729.529 | 157.6032 | 642.4974 | |
| 618.311 | 415.1874 | 370.1774 | 322.3854 | 236.66 | |
| 621.5428 | 451.1357 | 941.5142 | 169.2962 | 484.1679 | |
| 452.061 | 380.0612 | 236.7774 | 163.1811 | 564.3697 | |
| 710.3695 | 408.2592 | 391.2743 | 246.6879 | 591.2698 | |
| 534.1764 | 1990.612 | 160.248 | 156.2575 | 282.8166 | |
| 812.246 | 1990.654 | 449.3272 | 157.8793 | 686.3228 | |
| 487.2512 | 1992.766 | 425.0911 | 216.1807 | 164.4096 | |
| 783.5662 | 659.1805 | 515.1195 | 369.1103 | 154.6974 | |
| 429.2563 | 575.35 | 321.0493 | 224.3363 | 218.1713 | |
| 773.4065 | 727.479 | 506.2421 | 178.9761 | 149.6476 | |
| 621.7522 | 563.1981 | 168.9802 | 149.8833 | 291.32 | |
| 446.2992 | 969.4921 | 415.2159 | 195.7255 | 714.8228 | |
| 887.5296 | 589.3127 | 861.5197 | 161.9198 | 201.0575 | |
| 399.1431 | 487.1877 | 261.6977 | 236.9036 | 175.6901 | |
| 1995.012 | 431.0919 | 292.4926 | 174.9428 | 202.7882 | |
| 468.9767 | 517.1977 | 508.1461 | 195.8828 | 162.6195 | |
| 1657.363 | 506.2243 | 186.1136 | 165.1064 | 404.0439 | |
| 451.0526 | 504.1856 | 272.1292 | 345.1667 | 416.2151 | |
| 529.7793 | 589.2869 | 313.2384 | 270.0317 | 164.4352 | |
| 168.0152 | 587.3226 | 499.2928 | 310.7544 | 153.2672 | |
| 522.3347 | 1994.901 | 479.1194 | 290.0883 | 344.1463 | |
| 589.2514 | 728.4826 | 280.2363 | 355.1549 | 379.8057 | |
| 1741.725 | 621.2312 | 379.0581 | 148.21 | 161.3089 | |
| 710.2645 | 557.3028 | 232.0592 | 156.1753 | 226.0554 | |
| 415.1751 | 1990.562 | 367.1034 | 356.8252 | 303.1148 | |
| 857.0105 | 351.0669 | 610.0255 | 347.9441 | 174.7661 | |
| 1381.029 | 559.352 | 587.1406 | 154.1084 | 404.2478 | |
| 1506.346 | 535.134 | 181.9918 | 159.555 | 372.4872 | |
| 460.7781 | 489.1269 | 162.2331 | 157.0759 | 167.4343 | |
| 301.1679 | 491.0418 | 472.1587 | 257.3163 | 204.0374 | |
| 636.3361 | 466.1102 | 473.2521 | 317.0203 | 322.7285 | |
| 867.4634 | 398.1315 | 551.1406 | 161.6406 | 341.1831 | |
| 248.6265 | 529.1616 | 316.0803 | 163.3331 | 283.2552 | |
| 300.9955 | 731.2792 | 450.3305 | 167.385 | 196.5932 | |
| 931.6651 | 665.1876 | 397.0776 | 211.1401 | 153.9986 | |
| 1591.043 | 729.1638 | 218.0856 | 221.0804 | 238.5565 | |
| 365.564 | 557.3256 | 323.6225 | 152.2745 | 205.1746 | |
| 399.1661 | 487.089 | 398.1932 | 270.0827 | 207.3042 | |
| 1199.166 | 1028.536 | 197.0456 | 158.0909 | 208.7803 | |
| 1373.979 | 429.2427 | 481.2629 | 152.1975 | 177.5874 | |
| 331.5172 | 706.1701 | 815.5573 | 349.8377 | 161.8125 | |
| 1226.765 | 405.2404 | 284.1252 | 168.4953 | 161.3079 | |
| 429.2693 | 160.1922 | 276.2485 | 165.3329 | 249.6988 | |
| 1085.517 | 556.3372 | 328.1513 | 163.7827 | 331.721 | |
| 547.2159 | 505.2757 | 811.5368 | 194.7627 | 160.4188 | |
| 1350.953 | 531.2238 | 475.2513 | 150.7408 | 364.8374 | |
| 676.7303 | 1023.98 | 342.1671 | 163.9911 | 161.9936 | |
| 472.2562 | 632.2041 | 248.6234 | 168.348 | 162.2116 | |
| 1057.775 | 590.7683 | 444.2812 | 163.5213 | 167.4609 | |
| 447.3144 | 518.1747 | 276.2524 | 159.1959 | 221.8585 | |
| 679.2317 | 236.7973 | 563.1406 | 164.9187 | 167.7502 | |
| 812.1161 | 1933.996 | 330.2367 | 248.6136 | 160.8668 | |
| 603.2155 | 1623.344 | 935.5269 | 207.5998 | 161.3115 | |
| 724.9151 | 944.7537 | 531.1144 | 215.3571 | 359.0996 | |
| 575.4535 | 488.7353 | 464.3099 | 163.0181 | 300.1241 | |
| 1109.252 | 591.3222 | 621.6481 | 163.1448 | 163.843 | |
| 710.3378 | 505.2952 | 442.2448 | 307.8014 | 190.1777 | |
| 557.1503 | 501.1666 | 475.2353 | 162.8711 | 163.0863 | |
| 851.5629 | 489.1059 | 342.1307 | 155.8071 | 160.3173 | |
| 472.2254 | 163.8582 | 353.0878 | 237.7054 | 165.797 | |
| 738.7412 | 1006.762 | 428.317 | 267.7849 | 161.082 | |
| 928.5499 | 533.0884 | 784.4919 | 171.3603 | 180.2423 | |
| 433.0776 | 1161.2 | 226.0134 | 289.7783 | 161.4412 | |
| 411.1061 | 522.1318 | 256.2362 | 214.7433 | 167.5065 | |

TABLE 4
List of biomarker determinants that correlate with neither CCD nor
ICD (each number is the m/z ratio of a specific biomarker determinant).

| | | | | | |
|---|---|---|---|---|---|
| 489.2622 | 452.0529 | 871.1204 | 740.2983 | 187.0534 | 345.1302 |
| 265.1479 | 816.7223 | 1988.516 | 651.1691 | 585.5654 | 166.6705 |
| 593.3344 | 417.1432 | 342.2399 | 757.3544 | 164.5506 | 168.7023 |
| 490.2656 | 469.2173 | 465.2354 | 151.8711 | 382.518 | 329.7393 |
| 329.103 | 260.1139 | 162.6963 | 520.4266 | 317.0848 | 315.2153 |
| 449.3124 | 579.2575 | 710.3582 | 1091.92 | 736.405 | 160.9616 |
| 325.1843 | 460.7917 | 803.3778 | 419.1687 | 350.1256 | 236.7976 |
| 311.1686 | 616.3197 | 353.2155 | 620.0826 | 606.711 | 290.3546 |
| 427.2621 | 473.2393 | 532.8417 | 568.1829 | 215.1061 | 329.4699 |
| 594.3377 | 408.9267 | 794.7403 | 398.8173 | 168.8069 | 181.0159 |
| 327.0874 | 242.1035 | 595.188 | 1066.183 | 678.1793 | 331.5979 |
| 595.35 | 366.156 | 216.2204 | 274.908 | 726.3477 | 204.4342 |
| 339.1999 | 328.1402 | 159.9524 | 298.1165 | 498.015 | 343.1287 |
| 423.2388 | 538.8078 | 165.7607 | 165.456 | 607.1454 | 169.0484 |
| 397.1868 | 399.1774 | 164.2292 | 395.1573 | 163.4084 | 155.4497 |
| 465.2857 | 558.7685 | 465.0471 | 713.3843 | 545.2074 | 210.9064 |
| 266.1513 | 751.1618 | 589.3545 | 780.2994 | 754.472 | 221.6443 |
| 427.0799 | 490.2194 | 207.201 | 1120.912 | 498.1621 | 238.7175 |
| 319.1663 | 638.3275 | 621.2057 | 575.1958 | 330.5412 | 167.2389 |
| 365.1969 | 200.3209 | 1994.976 | 226.0244 | 544.1631 | 313.86 |
| 459.0698 | 391.2489 | 160.411 | 879.4613 | 514.9507 | 168.7245 |
| 456.8461 | 603.0423 | 860.6861 | 619.3304 | 537.1251 | 205.1423 |
| 497.3484 | 499.2736 | 458.8503 | 226.0442 | 498.5276 | 159.4642 |
| 691.3113 | 311.1616 | 693.5098 | 784.5985 | 419.1793 | 321.1147 |
| 473.2673 | 266.1473 | 548.0419 | 452.1306 | 206.7995 | 201.669 |
| 421.2959 | 667.302 | 658.7663 | 163.2679 | 534.2571 | 258.6336 |
| 377.2729 | 429.2686 | 455.2511 | 482.2915 | 546.2822 | 226.666 |
| 285.1132 | 519.2236 | 445.127 | 543.8659 | 602.7387 | 161.2928 |
| 243.1238 | 613.1831 | 226.0495 | 614.3528 | 624.4968 | 184.3237 |
| 429.2591 | 315.1965 | 355.2075 | 609.7219 | 211.9139 | 199.774 |
| 421.2595 | 591.0025 | 383.2568 | 195.4137 | 168.7073 | 316.4033 |
| 450.3157 | 236.7947 | 352.9034 | 221.0689 | 642.3182 | 161.323 |
| 481.2806 | 265.1529 | 753.3856 | 630.3106 | 379.1283 | 342.0764 |
| 349.202 | 330.1194 | 395.2107 | 276.2752 | 828.7265 | 161.0428 |
| 373.2748 | 674.3489 | 434.7371 | 526.3914 | 646.0001 | 164.1277 |
| 596.3534 | 241.1445 | 519.2284 | 768.4526 | 641.1191 | 153.343 |
| 439.2835 | 341.1606 | 657.165 | 509.2236 | 535.2553 | 201.8562 |
| 428.2654 | 607.2054 | 703.5156 | 470.2624 | 606.2892 | 301.2012 |
| 229.1445 | 621.5452 | 537.0971 | 1051.671 | 364.0755 | 153.5555 |
| 468.214 | 561.2116 | 592.0059 | 585.0057 | 182.4075 | 161.5898 |
| 336.8873 | 397.1253 | 449.2041 | 783.4969 | 417.7839 | 307.7945 |
| 457.2571 | 433.2231 | 598.9945 | 1099.984 | 375.1709 | 154.4429 |
| 297.153 | 176.3594 | 472.0472 | 431.5579 | 157.4977 | 156.113 |
| 588.4667 | 762.7094 | 236.7913 | 346.7563 | 420.4672 | 162.6401 |
| 369.1918 | 241.524 | 627.0588 | 1301.203 | 161.6673 | 227.3323 |
| 330.1064 | 449.2827 | 1992.863 | 364.8562 | 437.7074 | 213.6683 |
| 515.2684 | 455.1815 | 328.2242 | 158.0627 | 187.2667 | 278.9319 |
| 437.2544 | 615.3208 | 593.3243 | 347.0879 | 399.1882 | 166.6114 |
| 513.3433 | 594.3304 | 565.1178 | 458.9213 | 440.2515 | 160.4052 |
| 527.2602 | 346.1126 | 506.9038 | 544.1937 | 571.1827 | 171.2207 |
| 481.3171 | 292.5023 | 531.2421 | 443.2017 | 355.1867 | 164.3116 |
| 411.2337 | 267.1521 | 1768.821 | 1451.041 | 698.6949 | 284.122 |
| 283.1187 | 452.2735 | 1994.884 | 584.3262 | 491.2164 | 276.6717 |
| 241.1081 | 229.1082 | 647.5075 | 156.6483 | 463.1713 | 171.9423 |
| 305.1507 | 267.1546 | 691.3073 | 159.8037 | 693.3489 | 159.6875 |
| 315.1238 | 528.8855 | 606.7953 | 531.1723 | 166.1577 | 159.4613 |
| 435.2752 | 428.1561 | 715.4915 | 503.1581 | 211.0947 | 266.2036 |
| 478.828 | 544.7738 | 382.5345 | 986.4182 | 626.9411 | 287.878 |
| 326.1876 | 450.0652 | 570.3258 | 267.1386 | 483.266 | 281.9017 |
| 576.8049 | 425.2454 | 434.2448 | 1293.802 | 165.9177 | 154.3352 |
| 312.172 | 385.1438 | 1992.667 | 418.3551 | 706.3059 | 189.1119 |
| 216.9285 | 490.2314 | 184.2953 | 523.1465 | 583.2581 | 312.0384 |
| 201.1132 | 393.1668 | 331.5373 | 155.8701 | 503.3578 | 180.3379 |
| 385.1066 | 443.0652 | 292.5249 | 391.2716 | 530.4299 | 336.8792 |
| 437.1453 | 244.1271 | 560.162 | 636.6811 | 172.7134 | 228.1281 |
| 363.1449 | 454.8257 | 923.4435 | 1063.999 | 300.1283 | 161.8166 |
| 483.2732 | 292.5029 | 299.2624 | 344.0638 | 702.332 | 165.8359 |
| 184.282 | 369.9953 | 378.1639 | 532.2135 | 403.1404 | 181.111 |
| 385.2231 | 528.235 | 830.9206 | 743.1536 | 524.8102 | 150.6994 |
| 420.2292 | 481.3357 | 523.2289 | 552.4967 | 651.3219 | 162.563 |
| 609.221 | 492.2895 | 489.1635 | 499.0986 | 661.9683 | 207.5894 |
| 369.2282 | 451.3191 | 248.6556 | 1023.644 | 364.5513 | 160.534 |
| 519.3327 | 515.2397 | 154.3904 | 705.2905 | 556.7991 | 198.8298 |
| 379.2126 | 393.2646 | 292.9234 | 599.775 | 207.4829 | 344.2126 |
| 607.3137 | 631.4806 | 480.8322 | 430.8402 | 494.793 | 213.6612 |
| 353.1969 | 609.9241 | 563.0788 | 481.165 | 445.0994 | 289.8949 |
| 503.3378 | 428.8586 | 265.1457 | 335.1307 | 697.2008 | 164.3169 |
| 328.0908 | 321.1707 | 749.1458 | 301.1071 | 155.5657 | 254.0704 |
| 451.2701 | 419.1097 | 325.0863 | 576.7561 | 520.5222 | 222.1005 |
| 469.2807 | 525.3254 | 442.2799 | 160.6217 | 397.052 | 164.2411 |
| 535.3276 | 642.7507 | 636.7848 | 626.8624 | 597.3279 | 329.3379 |
| 187.0976 | 1992.854 | 701.311 | 1465.269 | 163.4808 | 256.1741 |
| 605.298 | 533.1226 | 494.8019 | 409.1691 | 310.8131 | 256.9195 |
| 314.9053 | 489.252 | 385.0849 | 506.8994 | 724.2668 | 216.9004 |
| 595.341 | 639.3398 | 266.1406 | 1507.857 | 161.2961 | 223.4709 |
| 259.1816 | 495.1619 | 617.0169 | 497.2609 | 160.1121 | 202.1082 |
| 611.345 | 345.1707 | 455.1924 | 520.2777 | 166.0169 | 156.7025 |
| 449.0618 | 480.1333 | 611.183 | 420.9658 | 170.7437 | 340.1126 |
| 424.2421 | 491.2262 | 236.8076 | 808.9378 | 543.3125 | 170.6205 |
| 418.2136 | 288.9679 | 498.8023 | 581.2026 | 359.068 | 285.8788 |
| 261.1608 | 534.3153 | 659.5469 | 276.2804 | 159.7451 | 198.9109 |
| 395.2438 | 158.7576 | 341.1395 | 659.8266 | 345.512 | 264.3529 |
| 323.1612 | 491.2179 | 570.7996 | 592.7697 | 724.5444 | 172.143 |
| 467.265 | 261.7202 | 1243.058 | 277.05 | 548.9598 | 155.2446 |
| 305.187 | 520.9022 | 710.4025 | 964.0357 | 160.8562 | 235.0142 |
| 395.1712 | 390.9012 | 509.2254 | 332.122 | 382.5444 | 211.3739 |
| 307.1914 | 238.1085 | 501.1589 | 813.3422 | 637.3567 | 171.0371 |
| 434.8641 | 325.1656 | 403.1433 | 583.1937 | 612.1361 | 158.0965 |
| 609.3294 | 452.2371 | 710.3593 | 437.0618 | 160.4099 | 156.7659 |
| 341.1969 | 567.1871 | 276.2744 | 884.9964 | 719.4761 | 179.3744 |
| 340.2032 | 317.212 | 403.3069 | 334.289 | 553.0976 | 168.3585 |
| 541.3746 | 344.1352 | 581.2422 | 919.1857 | 155.3933 | 192.1412 |
| 631.2031 | 479.1405 | 483.104 | 336.8704 | 465.6505 | 155.0471 |
| 371.2439 | 299.2017 | 456.831 | 208.0492 | 652.204 | 232.4243 |
| 267.1437 | 310.5298 | 516.8262 | 560.2618 | 163.6795 | 180.0542 |
| 301.202 | 386.8383 | 391.0971 | 261.7133 | 161.1452 | 324.6894 |
| 335.1976 | 633.293 | 503.0336 | 497.2543 | 479.7042 | 219.4641 |
| 351.1813 | 514.2195 | 325.1767 | 874.8844 | 782.3968 | 150.2281 |
| 523.3276 | 1992.857 | 497.277 | 540.9247 | 403.185 | 150.8821 |
| 692.3146 | 563.2366 | 637.4602 | 1267.171 | 310.7874 | 183.0811 |
| 501.2469 | 438.1485 | 721.4297 | 512.1775 | 374.0258 | 176.4509 |
| 450.3119 | 653.3192 | 516.2026 | 434.8517 | 424.1362 | 158.675 |
| 335.1864 | 184.3108 | 435.0462 | 1253.786 | 155.1651 | 165.5117 |
| 466.2891 | 388.2057 | 1768.746 | 455.1821 | 463.1849 | 188.5889 |
| 421.1381 | 451.3151 | 778.322 | 1067.239 | 205.9202 | 184.3089 |
| 598.7868 | 226.0433 | 248.652 | 496.0132 | 659.1239 | 155.0587 |
| 353.2333 | 558.2455 | 531.2761 | 583.3867 | 693.5267 | 283.25 |
| 635.3086 | 441.1516 | 261.1604 | 890.9999 | 175.8914 | 208.036 |
| 393.2282 | 161.877 | 292.5123 | 617.6925 | 643.3307 | 160.7796 |
| 351.085 | 170.6909 | 327.1909 | 727.5151 | 160.0601 | 177.2393 |
| 451.2337 | 268.147 | 1769.95 | 503.1574 | 226.6245 | 328.5512 |
| 445.1503 | 516.9081 | 261.7399 | 160.1157 | 161.7184 | 168.4049 |
| 351.1449 | 503.2035 | 571.2525 | 741.0704 | 669.2869 | 164.2369 |
| 693.3269 | 465.1553 | 729.1799 | 635.9202 | 677.2871 | 149.6121 |
| 458.1456 | 710.3354 | 580.235 | 412.9607 | 399.8932 | 161.4392 |
| 398.1901 | 394.2317 | 533.0347 | 500.9391 | 154.706 | 258.1642 |
| 615.3163 | 455.1281 | 552.5431 | 670.8262 | 450.7679 | 155.9994 |
| 299.1248 | 511.265 | 433.1996 | 920.0063 | 671.5048 | 165.0265 |
| 574.451 | 362.8404 | 595.0344 | 861.7882 | 397.1982 | 202.9165 |
| 521.2159 | 516.235 | 339.1836 | 839.1423 | 682.2116 | 320.2224 |
| 419.2186 | 262.1641 | 492.2465 | 151.9 | 435.9786 | 150.8752 |
| 425.1454 | 160.4048 | 1988.458 | 457.9159 | 659.467 | 197.3297 |
| 441.2806 | 318.8509 | 518.2344 | 581.801 | 171.9134 | 165.85 |
| 347.15 | 594.2866 | 694.5131 | 349.2383 | 643.1849 | 315.5087 |
| 498.3517 | 1994.988 | 644.326 | 465.1019 | 374.1609 | 273.9399 |
| 415.0799 | 265.1579 | 573.3199 | 474.1887 | 321.9894 | 155.1594 |
| 491.3377 | 306.8978 | 414.3769 | 292.539 | 429.2908 | 229.4135 |
| 474.2707 | 802.3741 | 442.8727 | 786.545 | 324.11 | 149.8551 |
| 554.8229 | 310.8087 | 752.1649 | 1379.716 | 216.2129 | 200.0384 |
| 265.1428 | 811.2701 | 532.2447 | 213.0412 | 441.2624 | 152.7894 |
| 349.1769 | 543.0633 | 715.3088 | 969.7429 | 662.314 | 286.3474 |
| 533.312 | 398.2265 | 584.8134 | 473.5051 | 337.1855 | 274.8403 |
| 497.2756 | 439.1723 | 659.1716 | 1069.377 | 400.7423 | 213.6672 |
| 477.2858 | 266.9053 | 615.2076 | 482.0553 | 386.6644 | 164.5202 |
| 616.4616 | 484.2765 | 677.4069 | 474.1129 | 670.1421 | 213.5403 |
| 265.138 | 329.0952 | 414.3895 | 997.8574 | 169.8504 | 257.7821 |
| 493.2807 | 557.3331 | 339.1828 | 485.1558 | 300.999 | 161.4148 |
| 525.3796 | 459.2236 | 478.6804 | 621.1824 | 203.6686 | 165.1359 |
| 349.1656 | 724.5464 | 543.1619 | 568.3178 | 154.2981 | 190.3356 |
| 467.1347 | 710.3405 | 457.1463 | 849.4852 | 165.3184 | 293.4753 |
| 711.4841 | 450.8407 | 602.7412 | 477.6985 | 206.2103 | 159.0125 |
| 289.1656 | 289.1809 | 276.2835 | 431.1947 | 244.2112 | 167.3643 |
| 500.2402 | 307.5399 | 331.5298 | 1344.099 | 266.9861 | 253.7485 |
| 287.15 | 248.6514 | 1119.927 | 292.5299 | 148.0924 | 209.7155 |
| 507.3326 | 477.1167 | 615.3357 | 503.1956 | 403.1141 | 318.2639 |
| 500.81 | 472.8225 | 619.9798 | 882.6683 | 154.285 | 169.9557 |
| 428.0832 | 469.1826 | 485.2981 | 425.1283 | 315.5267 | 163.7839 |
| 422.2993 | 595.3377 | 452.0354 | 795.0719 | 160.1887 | 154.0758 |
| 525.3432 | 276.2765 | 516.2724 | 619.8756 | 293.3999 | 186.3352 |
| 346.1013 | 453.1515 | 582.3362 | 164.7259 | 195.0909 | 150.604 |
| 433.2959 | 384.1775 | 456.8168 | 732.9435 | 407.1158 | 180.3673 |
| 589.47 | 396.8429 | 495.3514 | 394.1296 | 427.0414 | 165.3292 |
| 341.1242 | 357.2645 | 522.8993 | 458.2146 | 153.62 | 152.7351 |
| 307.1663 | 261.7354 | 440.8069 | 638.1263 | 655.9506 | 259.0433 |
| 461.2908 | 164.957 | 1229.557 | 934.2888 | 292.527 | 292.1174 |
| 333.1353 | 379.1875 | 553.133 | 631.0224 | 160.9336 | 170.106 |
| 349.1404 | 227.0351 | 604.4452 | 1301.599 | 162.8478 | 164.4474 |
| 431.1195 | 426.2497 | 483.0413 | 994.4735 | 434.3775 | 173.1732 |
| 320.1696 | 334.1386 | 508.2415 | 495.1357 | 370.7621 | 152.0195 |
| 373.2596 | 452.3126 | 370.2077 | 522.8553 | 415.8372 | 305.0194 |
| 271.1551 | 159.9594 | 555.281 | 216.2192 | 178.1534 | 158.2019 |
| 367.1762 | 481.183 | 230.096 | 912.0151 | 227.7958 | 163.0305 |
| 337.202 | 355.2066 | 476.0796 | 510.1413 | 364.2791 | 308.3365 |
| 366.2003 | 706.5348 | 586.3109 | 405.2237 | 148.8084 | 226.6039 |
| 315.1813 | 1992.772 | 473.2825 | 159.9977 | 161.5402 | 219.6806 |
| 449.2544 | 567.3146 | 747.41 | 162.5089 | 377.9655 | 320.5635 |
| 311.1249 | 401.1227 | 622.9925 | 1070.815 | 384.1016 | 267.1359 |
| 425.0643 | 838.7044 | 248.6527 | 519.3073 | 172.0009 | 150.1998 |
| 495.2492 | 641.3153 | 723.0012 | 776.3579 | 165.0505 | 233.3672 |
| 358.8692 | 543.1872 | 600.2999 | 676.0515 | 233.8866 | 201.2016 |
| 245.1394 | 509.3611 | 521.2076 | 590.284 | 222.5143 | 178.6351 |
| 471.233 | 458.8885 | 675.1287 | 447.179 | 284.4405 | 168.0487 |
| 509.1453 | 542.2511 | 538.7838 | 1336.093 | 198.7276 | 160.7407 |
| 299.1864 | 359.1347 | 513.1818 | 664.7534 | 412.7716 | 206.0756 |
| 482.284 | 465.0567 | 1773.273 | 565.2731 | 161.6633 | 159.5023 |
| 451.3093 | 292.5048 | 443.2874 | 291.1991 | 393.1937 | 202.2443 |
| 505.317 | 207.1917 | 1926.67 | 529.1359 | 170.1252 | 157.5549 |
| 417.2032 | 276.2491 | 313.1752 | 427.2685 | 311.1757 | 159.5128 |
| 581.1664 | 248.1292 | 586.2217 | 515.1914 | 306.0311 | 163.4153 |
| 394.1224 | 489.2703 | 489.2165 | 319.0914 | 164.0274 | 167.9692 |
| 713.2931 | 641.44 | 1272.072 | 649.2877 | 236.8117 | 189.1543 |
| 637.3242 | 680.4255 | 593.3197 | 638.454 | 172.3241 | 307.2143 |
| 453.1991 | 274.166 | 710.3713 | 355.1799 | 216.2022 | 163.2078 |
| 448.8386 | 461.1217 | 447.1271 | 548.1595 | 229.1804 | 320.3705 |
| 673.3455 | 276.2758 | 518.9429 | 594.8639 | 261.2751 | 159.4024 |
| 284.1139 | 540.188 | 1197.603 | 758.7978 | 236.8042 | 163.2983 |
| 443.3014 | 378.1389 | 631.3385 | 320.1221 | 395.5386 | 160.4489 |
| 257.1659 | 423.2133 | 487.0595 | 472.2007 | 316.4147 | 158.5832 |
| 422.2629 | 578.969 | 323.1687 | 959.6589 | 388.8357 | 240.3197 |
| 505.0654 | 367.0799 | 523.1474 | 548.8174 | 150.638 | 171.2043 |
| 457.1354 | 184.2496 | 337.1843 | 213.0783 | 323.3512 | 327.0613 |
| 381.1555 | 186.939 | 466.2924 | 435.1552 | 171.4685 | 155.2503 |
| 430.2624 | 362.2265 | 369.2419 | 607.2298 | 165.7484 | 182.3443 |
| 516.1511 | 319.1551 | 504.231 | 214.3744 | 184.1723 | 168.2682 |
| 347.2439 | 274.8871 | 1252.113 | 495.0773 | 167.5722 | 236.4868 |
| 579.1508 | 330.8818 | 1995.029 | 478.1399 | 396.0775 | 148.9303 |
| 481.0517 | 160.2444 | 331.504 | 162.6634 | 161.4295 | 162.7325 |
| 417.2494 | 287.1136 | 248.6544 | 855.8677 | 383.1702 | 275.2871 |
| 403.2337 | 177.5977 | 555.2211 | 527.121 | 162.3856 | 189.7442 |
| 458.2604 | 521.2756 | 449.2254 | 602.1875 | 388.9861 | 160.5237 |
| 343.0605 | 162.5654 | 190.9796 | 1255.362 | 204.1127 | 278.0412 |
| 389.2545 | 450.2579 | 495.2955 | 634.1691 | 216.1928 | 208.0649 |
| 521.3119 | 331.5123 | 156.1826 | 236.8126 | 385.1406 | 161.8452 |
| 630.4772 | 457.1926 | 335.2247 | 507.1655 | 250.5499 | 155.9807 |
| 593.2834 | 1992.622 | 414.4202 | 153.7881 | 157.2743 | 160.7791 |
| 498.2245 | 549.2526 | 1461.716 | 534.3099 | 207.182 | 148.8657 |
| 159.0663 | 645.3098 | 700.7089 | 422.7462 | 164.7284 | 173.2106 |
| 359.0983 | 369.241 | 564.5381 | 162.0296 | 150.3151 | 160.3334 |
| 406.1507 | 687.5784 | 420.8511 | 308.9859 | 163.6052 | 285.2898 |
| 516.2716 | 160.4852 | 367.1891 | 438.2214 | 169.1772 | 169.8415 |
| 549.3068 | 603.4418 | 261.7409 | 261.7421 | 292.4433 | 217.8456 |
| 289.1922 | 472.8109 | 772.759 | 431.0746 | 365.4896 | 237.7573 |
| 620.7687 | 357.2047 | 431.1323 | 163.7185 | 284.8082 | 171.47 |
| 489.2857 | 531.1121 | 450.6401 | 988.434 | 345.186 | 289.6235 |
| 593.309 | 165.7556 | 649.259 | 547.1726 | 160.2723 | 159.5611 |
| 378.2762 | 232.9445 | 499.1195 | 1112.972 | 342.613 | 150.4461 |
| 325.202 | 656.7452 | 436.0159 | 385.0646 | 161.7713 | 327.56 |
| 440.2869 | 436.8402 | 671.3371 | 828.5913 | 166.7252 | 322.3285 |
| 453.094 | 568.7973 | 474.2569 | 559.0839 | 162.7738 | 197.9503 |
| 500.204 | 688.9795 | 435.2248 | 993.7411 | 163.7847 | 159.8341 |
| 455.156 | 450.8291 | 844.3916 | 1056.939 | 211.4724 | 270.4115 |
| 583.182 | 368.0686 | 641.1877 | 236.7935 | 158.6755 | 157.427 |
| 327.1197 | 184.2487 | 430.9088 | 659.2319 | 329.9955 | 153.1266 |
| 571.3487 | 482.7994 | 1988.508 | 434.7386 | 170.3186 | 231.1001 |
| 378.2034 | 419.0722 | 509.0367 | 993.9349 | 161.8164 | 150.8932 |
| 216.0877 | 164.6035 | 408.8462 | 615.2672 | 156.5264 | 266.3317 |
| 543.2632 | 355.2083 | 480.9485 | 503.2423 | 156.8055 | 235.0976 |
| 612.3483 | 483.0834 | 491.1932 | 867.8389 | 162.232 | 174.059 |
| 514.3466 | 600.8294 | 710.7384 | 1226.167 | 214.7025 | 163.707 |
| 603.2824 | 261.7344 | 685.5629 | 388.2922 | 253.8351 | 171.9473 |
| 696.7636 | 472.82 | 514.8047 | 341.2066 | 169.6485 | 175.8078 |
| 328.8797 | 602.4474 | 1675.256 | 1138.237 | 207.208 | 161.5227 |
| 286.1166 | 666.9975 | 521.2392 | 499.228 | 271.1546 | 292.497 |
| 460.0731 | 1990.63 | 551.1194 | 634.0568 | 207.1927 | 207.1931 |
| 343.1511 | 437.2309 | 871.6087 | 216.2167 | 164.5269 | 151.2764 |
| 796.5346 | 405.2031 | 574.1927 | 1166.115 | 361.1617 | 169.1422 |
| 512.1562 | 570.7877 | 445.0624 | 547.1067 | 165.8957 | 172.5583 |
| 426.8566 | 522.8132 | 576.2674 | 588.5899 | 251.8968 | 190.4205 |
| 364.1401 | 493.0258 | 356.2557 | 784.9219 | 213.1132 | 161.9091 |
| 342.1195 | 593.2317 | 458.8221 | 619.3518 | 382.5187 | 177.5998 |
| 421.2326 | 278.8584 | 784.6926 | 552.2318 | 366.6405 | 218.6976 |
| 408.1412 | 1988.562 | 302.1116 | 562.8774 | 167.7239 | 151.8049 |
| 396.8671 | 543.2825 | 431.1274 | 173.4954 | 164.8598 | 213.2127 |
| 426.1882 | 237.1261 | 832.9469 | 766.5608 | 265.1683 | 219.1057 |
| 491.2664 | 487.2006 | 679.4179 | 1112.976 | 259.5447 | 157.3578 |
| 319.1299 | 429.0956 | 828.7368 | 160.6076 | 161.6356 | 162.2377 |
| 381.1303 | 244.8976 | 292.5286 | 606.2774 | 357.8617 | 161.1239 |
| 395.1459 | 395.0748 | 310.817 | 338.8916 | 159.7932 | 155.3129 |
| 391.2126 | 1990.679 | 1769.882 | 293.068 | 329.0986 | 164.8684 |
| 435.1538 | 310.8106 | 585.2292 | 789.449 | 183.7172 | 196.0126 |
| 466.1355 | 721.5447 | 425.1921 | 436.8683 | 155.4429 | 216.2338 |
| 407.1459 | 391.1119 | 405.2143 | 991.4186 | 163.899 | 216.7174 |
| 418.1985 | 608.5152 | 650.968 | 1223.882 | 156.7097 | 197.9395 |
| 209.068 | 585.2186 | 564.1874 | 398.1099 | 152.2311 | 165.022 |
| 452.1563 | 358.1507 | 906.0471 | 244.119 | 162.2007 | 160.5886 |
| 471.0438 | 599.1977 | 1133.861 | 379.1567 | 204.3126 | 163.9752 |
| 471.2137 | 710.9615 | 621.3379 | 619.4241 | 195.1887 | 186.8335 |
| 276.1088 | 641.3191 | 498.1882 | 1096.742 | 377.1255 | 157.4315 |
| 459.166 | 508.336 | 238.9104 | 458.8488 | 215.5398 | 166.2515 |
| 438.2578 | 472.1724 | 458.2476 | 150.1269 | 171.471 | 222.3749 |
| 608.317 | 427.2363 | 602.7585 | 445.2226 | 163.5067 | 176.7272 |
| 248.6356 | 506.0687 | 450.3206 | 415.4237 | 307.154 | 201.3063 |
| 470.8204 | 633.4917 | 489.3261 | 702.2283 | 302.1706 | 209.5 |
| 525.2446 | 375.0867 | 897.3581 | 628.7572 | 361.0672 | 167.413 |
| 340.8586 | 591.1844 | 337.2054 | 468.2827 | 159.5887 | 244.549 |
| 339.1813 | 482.1778 | 654.1876 | 168.2302 | 372.1664 | 160.1191 |
| 425.2464 | 421.176 | 1033.168 | 168.5743 | 161.5918 | 162.2481 |
| 350.2053 | 226.0448 | 444.1408 | 211.9935 | 244.0905 | 297.4958 |
| 482.2296 | 306.1194 | 663.235 | 1235.873 | 167.9976 | 165.6396 |
| 454.1507 | 418.8248 | 434.1384 | 397.1645 | 216.2217 | 279.1706 |
| 329.1242 | 530.2142 | 597.4133 | 1076.563 | 226.2897 | 161.18 |
| 436.315 | 497.2624 | 1066.086 | 939.5351 | 156.9055 | 163.7858 |
| 431.1823 | 543.1975 | 549.029 | 887.5555 | 169.6132 | 161.4304 |
| 461.193 | 449.2977 | 489.2966 | 606.7444 | 169.9295 | 195.9066 |
| 641.3555 | 266.1462 | 355.218 | 448.1915 | 190.022 | 164.6746 |
| 419.2438 | 420.2142 | 560.7658 | 453.1668 | 166.0151 | 197.6119 |
| 265.1373 | 553.3115 | 614.7608 | 487.0548 | 318.3683 | 203.3665 |
| 718.7456 | 515.2416 | 1883.723 | 557.2841 | 153.1018 | 165.8612 |
| 410.1457 | 554.2882 | 479.1498 | 458.8377 | 163.0377 | 178.7949 |
| 464.8546 | 1994.804 | 653.2059 | 1101.666 | 171.1239 | 172.7093 |
| 629.1875 | 647.0062 | 528.2192 | 157.4998 | 171.0923 | 165.7071 |
| 463.2086 | 423.2257 | 397.1858 | 1092.522 | 161.8102 | 168.123 |
| 357.1667 | 408.1665 | 1372.131 | 331.5421 | 359.1556 | 272.0229 |
| 481.1515 | 585.2566 | 441.205 | 338.1109 | 276.2848 | 183.9193 |
| 475.2085 | 261.7368 | 592.1869 | 711.1434 | 246.2446 | 194.1876 |
| 470.2839 | 333.1819 | 557.269 | 1015.654 | 219.3096 | 156.5857 |
| 369.2047 | 331.5001 | 1619.074 | 1056.766 | 195.9124 | 159.4151 |
| 488.3462 | 374.8852 | 1050.313 | 341.3031 | 290.4851 | 155.1809 |
| 597.3568 | 652.7999 | 619.8741 | 903.5043 | 208.5307 | 163.2538 |
| 355.2126 | 404.8747 | 427.0643 | 565.0207 | 161.3581 | 232.2438 |
| 482.3204 | 627.2479 | 454.2672 | 490.0993 | 268.8278 | 154.0736 |
| 447.0461 | 359.1612 | 184.295 | 1129.721 | 148.5924 | 206.0925 |
| 449.1929 | 325.1035 | 554.2945 | 614.7518 | 270.1769 | 207.1974 |
| 509.3484 | 241.5247 | 480.0392 | 522.1773 | 250.5043 | 155.8539 |
| 617.3319 | 387.1025 | 513.3043 | 516.9033 | 161.4661 | 159.8759 |
| 528.2716 | 461.2074 | 948.9592 | 456.3314 | 163.4654 | 165.8283 |
| 463.1739 | 532.1936 | 310.7963 | 521.2618 | 248.6578 | 163.2809 |
| 478.2155 | 578.7811 | 545.2936 | 469.5824 | 369.9857 | 171.1349 |
| 356.0988 | 355.2036 | 414.3721 | 1295.883 | 171.4823 | 164.4545 |
| 518.3204 | 479.2188 | 222.0805 | 751.261 | 171.6541 | 172.4902 |
| 254.9264 | 485.1514 | 713.476 | 456.8227 | 155.2754 | 207.2266 |
| 340.1401 | 1990.676 | 1657.526 | 596.4056 | 289.2672 | 218.6826 |
| 610.3328 | 1992.803 | 609.2373 | 160.9194 | 173.6171 | 158.3652 |
| 288.8872 | 779.6047 | 519.2567 | 231.009 | 171.358 | 219.2445 |
| 636.3119 | 469.11 | 576.1734 | 445.2209 | 247.1187 | 178.4476 |
| 335.1612 | 751.3696 | 777.6872 | 413.2087 | 236.7873 | 254.1824 |
| 312.1452 | 352.0882 | 543.3145 | 551.2493 | 159.5194 | 151.3305 |
| 377.1718 | 547.3122 | 649.1553 | 419.1682 | 152.5266 | 230.3715 |
| 724.5506 | 426.0678 | 430.2313 | 490.0998 | 310.7104 | 164.8563 |
| 171.4378 | 382.5 | 455.1913 | 195.0535 | 370.0887 | 223.2089 |
| 463.1722 | 355.1966 | 642.3467 | 605.1874 | 159.9905 | 155.945 |
| 522.7919 | 312.8849 | 605.1668 | 163.395 | 230.1 | 157.359 |
| 601.2135 | 276.269 | 348.1929 | 572.2265 | 216.7874 | 161.554 |
| 579.2315 | 444.3071 | 1253.451 | 966.3833 | 190.5276 | 164.153 |
| 298.1296 | 441.2906 | 475.2362 | 886.6412 | 309.1531 | 205.1984 |
| 305.1758 | 511.2043 | 424.2168 | 545.1049 | 150.1889 | 152.7968 |
| 602.446 | 572.1502 | 1495.915 | 548.8577 | 163.3516 | 150.8618 |
| 447.166 | 450.1519 | 1657.344 | 552.5182 | 210.5208 | 166.7089 |
| 445.1537 | 408.097 | 482.155 | 849.6518 | 157.5078 | 148.9836 |
| 173.0819 | 489.2982 | 1839.7 | 453.0851 | 159.7959 | 168.106 |
| 380.1431 | 564.2657 | 414.3798 | 1076.725 | 231.4028 | 212.8865 |
| 694.3303 | 236.8029 | 479.1281 | 526.3794 | 168.9913 | 179.4786 |
| 357.1304 | 454.0974 | 239.5236 | 827.7222 | 154.5434 | 155.9824 |
| 493.2191 | 382.2315 | 1769.672 | 1094.586 | 305.2489 | 169.6054 |
| 362.0507 | 310.7849 | 569.8478 | 452.0953 | 159.9628 | 163.7309 |
| 563.2627 | 159.2922 | 723.3364 | 997.1598 | 150.975 | 152.9888 |
| 476.1674 | 563.024 | 557.1524 | 484.8582 | 278.2147 | 147.7297 |
| 553.2629 | 452.0439 | 382.536 | 921.0362 | 164.7429 | 170.2673 |
| 501.2243 | 924.4481 | 355.2006 | 856.4941 | 347.8732 | 158.3126 |
| 436.2785 | 513.3255 | 322.848 | 437.6485 | 351.7725 | 208.5633 |
| 485.2292 | 511.2058 | 511.0552 | 837.2669 | 161.8375 | 164.5621 |
| 535.2734 | 207.2069 | 589.3146 | 479.1372 | 201.6024 | 172.4769 |
| 271.1914 | 449.1202 | 523.1439 | 248.6385 | 168.6516 | 150.2678 |
| 610.2244 | 442.183 | 226.0503 | 389.1752 | 167.7646 | 169.8113 |
| 407.1824 | 236.7895 | 439.2551 | 627.1724 | 202.2185 | 190.4876 |
| 380.8511 | 405.2494 | 1896.467 | 478.7943 | 160.5306 | 171.4724 |
| 322.1296 | 382.5267 | 638.7802 | 447.1086 | 369.823 | 206.4967 |
| 536.7866 | 669.2007 | 610.2254 | 495.274 | 314.1115 | 160.45 |
| 426.1406 | 597.8536 | 755.7538 | 531.1879 | 159.1199 | 169.3832 |
| 308.8999 | 396.1664 | 423.2491 | 397.1768 | 156.3582 | 150.9808 |
| 403.1874 | 363.1248 | 464.1769 | 496.346 | 171.5784 | 155.1504 |
| 465.1989 | 517.1454 | 1424.724 | 453.7259 | 166.5679 | 163.4043 |
| 468.2683 | 624.7182 | 631.277 | 372.1878 | 354.502 | 150.1116 |
| 419.1824 | 1226.901 | 159.5633 | 488.5459 | 158.9349 | 207.0569 |
| 326.0834 | 163.2142 | 382.2631 | 476.4802 | 337.266 | 163.3445 |
| 383.0748 | 248.628 | 1797.614 | 386.347 | 294.7705 | 154.0992 |
| 1990.693 | 571.1995 | 596.2512 | 216.2177 | 284.4439 | 164.1329 |
| 569.0207 | 1229.969 | 902.7772 | 1230.848 | 302.0703 | 164.8544 |
| 465.1875 | 532.1827 | 265.1544 | 1008.801 | 166.0247 | 230.7427 |
| 493.3534 | 515.2156 | 248.6465 | 1197.435 | 295.4903 | 257.506 |
| 394.8458 | 162.6197 | 665.1832 | 508.2052 | 195.047 | 230.473 |
| 655.3348 | 259.1551 | 1156.823 | 928.3048 | 352.8264 | 150.5906 |
| 521.1093 | 1769.471 | 585.0495 | 442.2307 | 209.8786 | 184.1762 |
| 433.1981 | 163.4518 | 972.3979 | 500.1921 | 305.4947 | 167.145 |
| 391.2885 | 226.0146 | 352.8637 | 552.5077 | 268.6787 | 209.2892 |
| 491.1668 | 228.0336 | 350.8654 | 461.1685 | 172.6337 | 169.4928 |
| 445.198 | 276.2731 | 422.1791 | 634.6353 | 162.791 | 216.4105 |
| 632.2063 | 463.041 | 438.1956 | 628.3312 | 307.2534 | 250.0937 |
| 416.8278 | 528.7791 | 557.1782 | 485.3038 | 162.8378 | 169.6104 |
| 306.154 | 309.1708 | 1711.628 | 1039.453 | 292.382 | 155.0074 |
| 623.3451 | 573.053 | 464.9319 | 497.6526 | 269.8335 | 160.5724 |
| 441.2129 | 531.1983 | 1977.281 | 203.641 | 155.7324 | 212.3439 |
| 293.0292 | 184.2468 | 1944.496 | 919.4248 | 165.9001 | 166.8806 |
| 679.4221 | 489.2494 | 630.9099 | 478.2348 | 230.4284 | 164.1594 |
| 399.141 | 236.8067 | 706.5299 | 1143.715 | 166.4099 | 165.8313 |
| 542.3779 | 643.2419 | 276.2823 | 510.7653 | 295.5714 | 152.1513 |
| 474.1769 | 598.1931 | 1040.589 | 211.8729 | 226.0531 | 198.2405 |
| 674.7817 | 592.7789 | 419.1604 | 557.4961 | 285.03 | 213.906 |
| 365.2333 | 161.586 | 460.1941 | 200.3968 | 216.7246 | 202.8895 |
| 460.8174 | 437.1922 | 593.1661 | 412.0542 | 226.446 | 174.6496 |
| 765.5673 | 550.8677 | 465.2994 | 579.2561 | 188.8696 | 160.8099 |
| 336.2261 | 799.3812 | 540.3039 | 349.7772 | 231.9661 | 163.4693 |
| 369.1667 | 417.1895 | 404.1755 | 964.8229 | 152.1891 | 149.5268 |
| 323.1065 | 619.6668 | 488.7833 | 174.8355 | 228.2533 | 161.5846 |
| 292.5044 | 341.1006 | 519.1919 | 615.7312 | 169.8582 | 160.6428 |
| 467.167 | 527.0475 | 945.1465 | 869.6624 | 184.4153 | 190.7002 |
| 367.244 | 294.1825 | 648.5108 | 347.2227 | 168.5636 | 174.4982 |
| 412.8822 | 651.3151 | 261.7085 | 160.3792 | 166.0395 | 220.8677 |
| 547.0388 | 425.2429 | 513.7691 | 879.8691 | 181.837 | 214.6114 |
| 384.2656 | 601.3201 | 643.1776 | 594.7322 | 207.2084 | 165.4925 |
| 502.2559 | 158.8533 | 605.0769 | 1151.289 | 157.0517 | 158.1348 |
| 322.9137 | 158.7976 | 579.0286 | 855.8427 | 153.193 | 182.027 |
| 400.1249 | 517.1826 | 393.1779 | 485.5088 | 361.9908 | 151.7033 |
| 475.0759 | 413.12 | 784.5689 | 363.0826 | 166.6657 | 155.0351 |
| 256.0826 | 744.9827 | 447.1917 | 390.1214 | 157.5022 | 164.4807 |
| 657.3504 | 266.1538 | 477.3435 | 437.0856 | 236.8238 | 156.2106 |
| 436.1725 | 488.2167 | 1096.568 | 813.4774 | 161.1904 | 201.7229 |
| 216.2028 | 368.2474 | 210.3749 | 842.6224 | 170.4446 | 155.1284 |
| 565.338 | 160.569 | 659.3673 | 339.1916 | 175.4871 | 201.5755 |
| 499.3066 | 354.1558 | 432.9093 | 794.6562 | 162.2928 | 164.5118 |
| 306.1904 | 276.2795 | 511.225 | 754.6707 | 209.4013 | |
| 336.201 | 162.2982 | 165.7537 | 651.9615 | 203.6691 | |
| 451.2087 | 535.2313 | 659.4537 | 791.3847 | 190.3875 | |
| 740.7275 | 570.024 | 456.1848 | 633.5173 | 265.1361 | |
| 477.1766 | 391.1907 | 414.4147 | 583.3099 | 151.5732 | |
| 308.1948 | 152.3107 | 398.1699 | 418.6302 | 350.6912 | |
| 386.11 | 163.0875 | 601.0108 | 867.7926 | 160.1782 | |
| 555.3126 | 266.1414 | 168.8907 | 494.2476 | 334.5182 | |
| 229.0983 | 160.4571 | 617.7959 | 625.3243 | 294.3952 | |
| 236.793 | 303.0986 | 906.0449 | 419.249 | 196.8068 | |
| 522.219 | 299.0707 | 1111.572 | 177.5915 | 331.1095 | |
| 559.1879 | 519.1309 | 811.0051 | 869.9008 | 177.5059 | |
| 577.2159 | 159.2852 | 1684.263 | 615.3916 | 362.7527 | |
| 518.9052 | 159.7477 | 621.2331 | 355.2113 | 180.7847 | |
| 420.1016 | 433.1746 | 927.1847 | 433.2457 | 161.4755 | |
| 514.2559 | 689.1611 | 171.4835 | 733.2603 | 330.5159 | |
| 432.1664 | 159.0278 | 1564.19 | 484.1072 | 245.0616 | |
| 412.2371 | 570.2057 | 496.2079 | 579.7051 | 169.3102 | |
| 501.2834 | 254.0129 | 167.1343 | 164.9861 | 149.1708 | |
| 438.8097 | 475.088 | 464.2806 | 382.5042 | 161.8485 | |
| 230.1034 | 226.0469 | 1039.451 | 226.0218 | 319.1345 | |
| 531.2105 | 310.5282 | 994.4772 | 382.3077 | 162.9986 | |
| 413.231 | 551.218 | 647.3196 | 526.2159 | 309.9767 | |
| 1990.477 | 560.191 | 939.4392 | 414.3754 | 202.5895 | |
| 479.2036 | 204.9571 | 699.111 | 320.1351 | 258.0382 | |
| 443.0748 | 678.7276 | 454.1222 | 823.6838 | 179.3971 | |
| 284.9159 | 331.5343 | 551.9304 | 226.6804 | 172.5009 | |
| 532.8409 | 160.7301 | 972.5061 | 151.5364 | 194.5332 | |
| 184.4129 | 160.2062 | 452.0838 | 451.1455 | 306.7238 | |
| 477.1878 | 625.2323 | 403.1639 | 524.1889 | 364.1375 | |
| 226.0721 | 710.3527 | 918.9893 | 459.1515 | 150.0598 | |
| 653.185 | 207.206 | 527.1925 | 441.9876 | 352.8611 | |
| 436.2089 | 216.2161 | 359.0582 | 458.1646 | 259.1252 | |
| 617.4648 | 501.0178 | 561.1402 | 157.5181 | 241.295 | |
| 380.216 | 691.5517 | 575.1779 | 284.0855 | 265.5251 | |
| 543.2348 | 158.1544 | 778.8242 | 826.0084 | 218.4276 | |
| 361.1141 | 648.8445 | 434.1199 | 513.5233 | 283.1435 | |
| 420.2835 | 158.6408 | 837.31 | 861.3085 | 325.192 | |
| 381.2597 | 158.1205 | 413.2407 | 341.7253 | 306.7863 | |
| 440.2831 | 459.3268 | 680.4428 | 522.4151 | 359.3931 | |
| 226.0213 | 578.8093 | 854.1181 | 163.3743 | 184.1582 | |
| 531.2962 | 487.2336 | 1692.561 | 156.8467 | 207.2054 | |
| 260.8923 | 160.2166 | 155.8352 | 426.1426 | 160.9514 | |
| 428.2554 | 161.7245 | 525.1249 | 300.9714 | 189.078 | |
| 442.2839 | 477.1635 | 1416.125 | 620.5018 | 173.71 | |
| 464.1198 | 629.1935 | 207.2102 | 498.3466 | 164.9696 | |
| 444.2789 | 602.2165 | 1585.081 | 578.4329 | 256.1554 | |
| 159.0661 | 647.2041 | 598.8542 | 248.6484 | 293.3928 | |
| 358.1143 | 207.2045 | 451.3341 | 782.047 | 256.1374 | |
| 625.0242 | 773.1438 | 246.8947 | 1127.756 | 208.9986 | |

The method can further comprise (c) treating the subject for CCD or ICD.

The invention provides for a method of treating a subject for CD comprising: (a) identifying a subject having CD using a method of the present invention, and (b) administering a therapeutically effective amount of a drug suitable for treating and/or ameliorating the symptom(s) of CD to the subject, and/or performing surgery on the subject. Such suitable drugs include: an antidiarrheal medicine (such as loperamide), an aminosalicylates (such as sulfasalazine or mesalamine), an antibiotics (such as ciprofloxacin or metronidazole), a corticosteroid (such as budesonide or prednisone), an immunomodulator (such as azathioprine (AZA), 6-mercaptopurine (6-MP), or methotrexate), and a tumor necrosis factor (TNF) antagonist (such as infliximab or Remicade®).

### REFERENCES CITED

1. Sartor R B (2006) Mechanisms of disease: Pathogenesis of Crohn's disease and ulcerative colitis. Nature Clin Practice Gastroenterol & Hepatology 3: 390-407.
2. Willing B, Halfvarson J, Dicksved J, Rosenquist M, Jarnerot G, Engstrand L, Tysk C and Jansson J K (2009) Twin studies reveal specific imbalances in the mucosa-associated microbiota of patients with ileal Crohn's disease. Inflamm. Bowel Dis. 15:653-660.
3. Nicholson J K, Holmes E, Wilson I D (2005) Gut microorganisms, mammalian metabolism and personalized health care. Nature Rev Microbiol 3:431-438.
4. Wikoff W R, Anfora A T, Liu J, Schultz P G, Lesley S A, Peters E C, Siuzdak G (2009) Metabolomics analysis reveals large effects of gut microflora on mammalian blood metabolites. Proc Natl Acad Sci USA 106: 3698-3703.
5. Li M, Wang B, Zhang M, Rantalainen M, Wang S, et al. (2008) Symbiotic gut microbes modulate human metabolic phenotypes. Proc Natl Acad Sci USA 105: 2117-2122.
6. Holmes E, Loo R L, Stamler J, Bictash M, Yap I K S, et al. (2008) Human metabolic phenotype diversity and its association with diet and blood pressure. Nature 453: 396-400.
7. Marchesi J R, Holmes H, Khan F, Kochhar S, Scanlan P, et al. (2007) Rapid and noninvasive metabonomic characterization of inflammatory bowel disease. J Proteome Res 6: 546-551.
8. Saric J, Wang Y, Li J, Coen M, Utzinger J, et al. (2008) Species variation in the fecal metabolome gives insight into differential gastrointestinal function. J Proteome Res 7: 352360.
9. Rossello-Mora R, Lucio M, Peña A, Brito-Echeverría J, López-López A, et al. (2008) Metabolic evidence for biogeographic isolation of the extremophilic bacterium Salinibacter ruber. ISME J 2: 242-253.
10. Chen J, Zhao X, Fritsche J, Yin P, Schmitt-Kopplin P, et al. (2008) Practical approach for the identification and isomer elucidation of biomarkers detected in a metabonomic study for the discovery of individuals at risk for diabetes by integrating the chromatographic and mass spectrometric information. Anal Chem 80: 1280-1289.
11. Halfvarson J, Bodin L, Tysk C, Lindberg E, Järnerot G (2003) Inflammatory bowel disease in a Swedish twin cohort: A long-term follow-up of concordance and clinical characteristics. Gastroenterol 124: 1767-1773.
12. Dicksved J, Halfvarson J, Rosenquist M, Jarnerot G, Tysk C, Apajalahti J et al. (2008). Molecular analysis of the gut microbiota of identical twins with Crohn's disease. ISME J 2: 716-727.
13. Harvey R F, Bradshaw J M (1980) A simple index of Crohn's-disease activity. Lancet 315: 514.
14. Rutgeerts P, Goboes K, Peeters M, Hiele M, et al (1991) Effect of faecal stream diversion on recurrence of Crohn's disease in the neoterminal ileum. Lancet 338: 771-774.
15. Suhre K, Schmitt-Kopplin P (2008) Masstrix: Mass translator into pathways. Nucleic Acids Research 36: W481-W484.
16. Legendre P, Legendre L (1998) Numerical Ecology. Amsterdam, Elsevier Science 132: 303304.
17. Dicksved J, Halfvarson J, Rosenquist M, Jarnerot G, Tysk C, Apajalahti J, Engstrand L, Jansson (2008) JK Molecular analysis of the gut microbiota of identical twins with Crohn's disease. ISME J. 2:716-727.
18. Magro F, Vieira-Coelho M A, Fraga S, Serrao M P, Veloso F T, et al. (2002) Impaired synthesis or cellular storage of norepinephrine, dopamine and 5-hydroxytryptamine in human inflammatory bowl disease. Dig Dis Sci 47: 216-224.
19. Magro F, Cunha E, Araujo F, Meireles E, Pereira P, et al. (2006) Dopamine D2 receptor polymorphisms in inflammatory bowel disease and the refractory response to treatment. Dig Dis Sci 51: 2039-2044.
20. Sokol H, Pigneur B, Watterlot L, Lakhdari O, et al. (2008) Faecalibacterium prausnitzii is an anti-inflammatory commensal bacterium identified by gut microbiota analysis of Crohn disease patients. Proc Natl Acad Sci USA 105: 16731-16736.
21. Burczynski M E, Peterson R L, Twine N C, Zuberek K A, Brodeur B J (2006). Molecular classification of crohn's disease and ulcerative colitis patients using transcriptional profiles in peripheral blood mononuclear cells. J Mol Diag 8: 51-61.
22. Vang T, Miletic A V, Arimura Y, Tautz L, et al. (2008) Protein tyrosine phosphatases in autoimmunity. Ann Rev Immunology 26: 29-55.
23. Beeken W L, (1976) Serum tryptophan in Crohn's-disease. Scand J Gastroenterol 11: 735740.
24. Baumgart M, Dogan B, Rishniw M, Weitzman G, Bosworth B, Yantiss R et al. (2007) Culture independent analysis of ileal mucosa reveals a selective increase in invasive Escherichia coli of novel phylogeny relative to depletion of Clostridiales in Crohn's disease involving the ileum. ISME J 1: 403-418.
25. Lapidus A, Akerlund J E, Einarsson C (2006) Gallbladder bile composition in patients with crohn's disease. World J Gastroenterol 12: 70-74.
26. Chen F, Ma L, Sartor R B, Li F, Xiong H, et al. (2002). Inflammatory-mediated repression of the rat ileal sodium-dependent bile acid transporter by c-fos nuclear translocation. Gastroenterol 123: 2005-2016.
27. Lowes S, Simmons N L (2001) Human intestinal cell monolayers are preferentially sensitive to disruption of barrier function from basolateral exposure to cholic acid: Correlation with membrane transport and transepithelial secretion. Pflugers Archiv-European J Physiology 443: 265-273.
28. Fernández-Bañares F, Esteve-Comas M, Mañé J, Navarro E, Bertrán X, et al. (1997) Changes in mucosal fatty acid profile in inflammatory bowel disease and in experimental colitis: A common response to bowel inflammation. Clinical Nutrition 16: 177-183.
29. Ramakers J D, Mensink R P, Schaart G, Plat J (2007) Arachidonic acid but not eicosapentaenoic acid (epa) and oleic acid activates nf-kappa b and elevates icam-1 expression in caco-2 cells. Lipids 42: 687-698.
30. Libioulle C, Louis E, Hansoul S, Sandor C, Farnir F, et al. (2007) Novel Crohn disease locus identified by genome-wide association maps to a gene desert on 5p13.1 and modulates expression of PTGER4. Plos Genet. 3: e58.
31. Sheibanie A F, Yen J H, Khayrullina T, Emig F, Zhang M, Tuma R, Ganea D (2007) The Proinflammatory Effect of Prostaglandin E2 in Experimental Inflammatory Bowel Disease Is Mediated through the IL-23 . . . IL-17 Axis. J. Immunolog. 178, 8138-8147.
32. Karlsson P C, Huss U, Jenner A, Halliwell B, Bohlin L, Rafter J J (2005) Human Fecal Water Inhibits COX-2 in Colonic HT-29 Cells: Role of Phenolic Compounds. J. Nutr. 135, 23432349.
33. Verberkmoes N C, Russell A L, Shah M, Godzik A, Rosenquist M, Halfvarson J, Lefsrud M G, Apajalahti J, Tysk C, Hettich R L, Jansson J K (2009) Shotgun metaproteomics of the human distal gut microbiota. ISME J. 3, 179-189.

Each of the above cited references is hereby specifically and individually incorporated in their entireties.

### Example 1

Here we used non-targeted metabolic profiling to determine the contribution of metabolites produced by the gut microbiota towards disease status of the host. Ion Cyclotron Resonance Fourier Transform Mass Spectrometry (ICR-FT/MS) was used to discern the masses of thousands of metabolites in fecal samples collected from 17 identical twin pairs, including healthy individuals and those with CD. Pathways with differentiating metabolites included those involved in the metabolism and or synthesis of amino acids, fatty acids, bile acids and arachidonic acid. Several metabolites were positively or negatively correlated to the disease phenotype and to specific microbes previously characterized in the same samples. Our data reveal novel differentiating metabolites for CD that may provide diagnostic biomarkers and/or monitoring tools as well as insight into potential targets for disease therapy and prevention.

### Patient Cohort

The selection criteria and patient information for the twins studied in this experiment have previously been described [11], [12]. Patient information, including responses to a questionnaire regarding the usage of antibiotics, non-steroidal anti-inflammatory drugs during the preceding 12 months, gastroenteritis within the preceding 3 months, and specific dietary habits have previously been reported [12]. Written informed consent was obtained and approved by the Swedish ethical committee. The sample cohort was comprised of 15 twin pairs, with 7 healthy twin pairs, 4 pairs that were discordant for predominantly colonic CD (CCD), 2 pairs that were discordant for predominantly ileal CD (ICD), 2 pairs that were concordant for ICD, and 2 pairs that were concordant for CCD. There were a total of 8 individuals with CCD (aged 20-70, mean 48), 6 individuals with ICD (aged 44-53, mean 49.8), 6 healthy individuals with a sick twin (HD) (ages 20-70, mean 51.8) and 14 healthy individuals with a healthy twin (HH). In the HH group, 5 pairs were children (ages 5-11, mean 7.4) and 2 pairs were of similar ages to diseased individuals (45-55 mean 50). All diseased individuals were in clinical remission according to the Harvey-Bradshaw score [13] with the exception of 2 individuals (10b and 15a). Both of these individuals had endoscopic recurrence scores below 2 [14] at colonoscopy suggesting that the Harvey-Bradshaw score did not indicate active CD. Fecal samples were placed in a freezer at −70° C. within 24 h of collection, until analysis. The use of human subjects for this study was approved by the Örebro County Ethical Committee (Dnr; 167/03).

### Preparation of Fecal Water

Fecal samples were diluted 1:60 (weight:volume) in cold (4° C.) 50 mM sodium phosphate buffer (pH 8.0). The suspension was mixed in a gyratory shaker at 120 rpm for 10 min at 4° C. and then centrifuged at 200×g for 10 min at 4° C. to pellet the debris. The supernatant was removed and centrifuged at 18,000×g for 10 min to pellet the bacteria. The supernatant was collected and the pH was lowered to approximately 4.5 by addition of 0.25 mL of 1% formic acid to each mL of supernatant, with a resulting final sample dilution of 1:75. The fecal water samples were frozen and stored frozen at −70° C. until analysis. The samples were extracted 20 min after they were thawed at room temperature by solid phase extraction (SPE) using 1 L cartridges filled with 100 mg of octadecy-bonded silica packing (Bakerbond, Mallinckrodt Baker, Griesheim, Germany) for desalination and deproteination of the sample. The cartridges were preconditioned with 2 mL of methanol and 2 mL of water acidified with 0.1% formic acid prior to the application of 0.5 mL of sample. The columns were washed with 0.5 mL of 0.1% formic acid and metabolites were eluted with 0.5 mL methanol.

### FT-ICR-MS Analysis

High-resolution mass spectra (resolution (m/z)/(m/z) of 500.000 at m/z 500 in full scan mode) were acquired on a Fourier Transform Ion Cyclotron Resonance Mass Spectrometer (Bruker, Bremen, Germany), equipped with a 12 Tesla superconducting magnet and an Apollo II ESI source. Samples were infused with the micro-electrospray source at a flow rate of 120 μL/h with a nebulizer gas pressure of 20 psi, and a drying gas pressure of 15 psi at 250° C. Negative and positive electrospray ionisation was used. Spectra were externally calibrated on clusters of arginine (m/z of 173.10440, 347.21607, 521.32775 and 695.43943) dissolved in methanol at a concentration of 10 mg/L; calibration errors in the relevant mass range were always below 0.1 ppm. Four MW time domains were applied in the mass range of 150-2000 m/z. The ion accumulation time in the ion source was set to 2 s and 200 scans were accumulated per sample. Before Fourier transformation of the time-domain transient, a sine apodization was performed. The raw data were processed with DataAnalysis 3.4 (Bruker Daltonik, Bremen) software that is hard-coded in the instrument. Peaks exceeding a threshold signal-to-noise ratio of 3 were exported to peak lists. The extracted spectra were aligned though in-house software.

### Identification of Differentiating Masses and Assignment to Metabolic Pathways

Different multivariate analysis techniques including principal component analysis (PCA), hierarchical cluster analysis (HCA) and partial least square discriminant analysis (PLS-DA) were combined to reduce the data sets into a series of optimized and interpretable objects. The study of contribution of the different variables (m/z in this case) was done though the analysis of the regression coefficients. The statistical analyses were done with SIMCA-P 11.5 (Umetrics, Umea, Sweden), SAS version 9.1 (SAS Institute Inc., Cary, N.C., USA) and the Statistical package “R” (v 1.8.1) for the heatmap visualization.

The masses with the highest regression coefficient (an arbitrary cut off value>0.0004) were chosen to be discriminant. Moreover these masses have a variable importance in the projection VIP>1 (where a VIP value of =1 is regarded as significant). The VIP is a computation of the influence of every x term in the model on the y variable (ICD, CCD, and Healthy). Larger VIP values indicate a greater influence of a term x on the y variable.

We used a PLS-DA model to evaluate the m/z that contributed to separation between healthy vs individuals with CCD and ICD. The model gave good values for Q²(cum)=0.6 and R²(Y)=0.9. However the permutation test applied with 100 permutations revealed a possible over fitting of the model. Thus the dataset was reduced excluding the masses with frequencies less than three and a PLS model with an orthogonal signal correction (OSC) was applied. The putative masses responsible for the metabolic differentiation were used to make queries in the KEGG database (Kyoto Encyclopedia of Genes and Genomes) through the MassTRIX software [15] including Homo sapiens and Bacteroides vulgatus as reference species. MassTRIX calls the KEGG/API (http://www.genome.jp/kegg/soap/) to generate pathway maps, where the annotated compounds and genes are highlighted using different colors-thus differentiating between organism-specific and extra-organism items [15]. The identification of certain metabolites as their exact masses in their given biological context was strategic in the context of searching for biomarkers for CD.

### Correlation of Metabolites to Microbial Profiles

The bacterial community profiles of the same fecal samples studied here have previously been reported [12]. To determine correlation between the microbial community composition and the metabolic profiles, distance matrices using Manhattan distances [16] for microbial and metabolic profiles were calculated independently, then Pearson correlation coefficients between the two distance matrices were calculated. Significances of the correlations were tested using the Mantel test with 1000 permutations. Cluster analysis of microbial and metabolic profiles was performed using binary data (Jaccard's similarity index). Moreover the relation between the metabolites and the microbial profiles was analyzed with a PLS model with OSC, the metabolites were used as explicative variables. The relation of the first ten explicative masses with the different bacteria was expressed with the Heatmap.

### Discussion

Due to the tremendous individual diversity in the composition of the gut microbiota in humans and the corresponding anticipated diversity in the metabolites they produce, we expected it to be challenging to correlate specific metabolites to CD. Therefore, in this study, we focused on a twin cohort, described previously [2], [17], that includes healthy twin pairs, concordant pairs (both twins have CD) and discordant pairs (one twin is healthy and the matched twin has CD). The patients with CD were further differentiated depending on whether inflammation was primarily localized in the ileum (ICD) or in the colon (CCD) [2], [17]. A particular value of this patient cohort was the availability of existing data about the microbial profiles in fecal samples [17] and biopsies [2] from the same individuals that enabled the possibility of correlation of metabolites to microbial populations. Our previous studies showed that there were significant differences in levels of several members of the microbial communities in the gut of individuals that had ICD compared to those with CCD or to healthy individuals; in particular dramatically lower abundances of Faecalibacterium prasnitzii and higher levels of Escherichia coli in individuals with ICD compared to the other two groups [2]. However, there was no clear distinction between the microbial community profiles in healthy individuals and those with CCD. Although the gut microbial communities of healthy twins were more similar to each other than to other individuals in the sample cohort, this similarity was no longer evident when comparing twin sets where one or both twins were sick [17].

In this study, we used several multivariate statistical approaches to analyze the metabolites present in the liquid phase (fecal water) of the same fecal samples examined earlier [17]. First, using principal components analysis (PCA) we found that the metabolomes of individuals that had ICD grouped separately from those with CCD and from healthy individuals (Supplementary Fig. S1; which is available on the internet at: “http://www.lbl.gov/tt/publications/2676pub.pdf”). Some of the healthy individuals were young and their metabolomes grouped separately from the healthy adults on the PCA plots. This distinction between healthy young and old was not evident in our previous analyses of the microbial community compositions [17]. An example of a discriminating metabolite that contributed highly to the grouping of the young was 5β-cyprinosulfate, a bile acid that was more abundant in young subjects (P<0.003) compared to all other groups. Because there were no young individuals with CD in this study, we continued with adults only for further statistical discrimination of diseased from healthy groups.

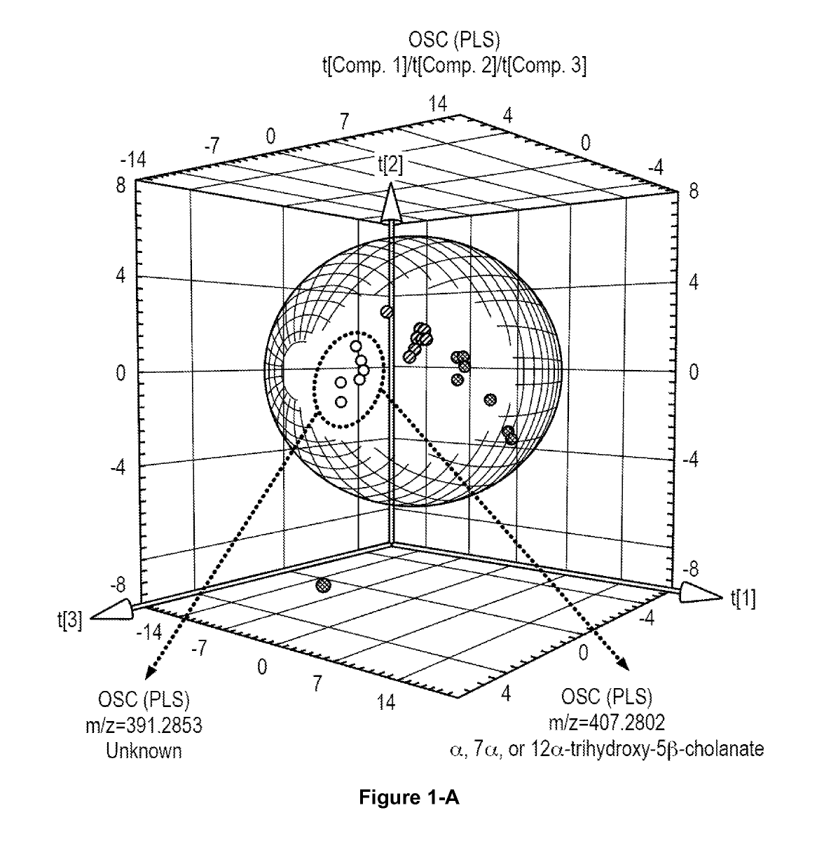

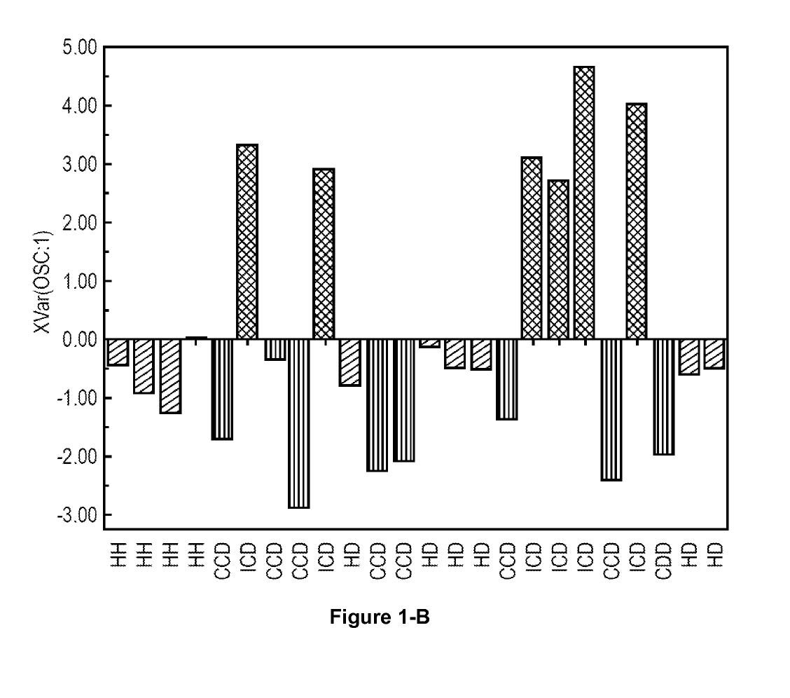

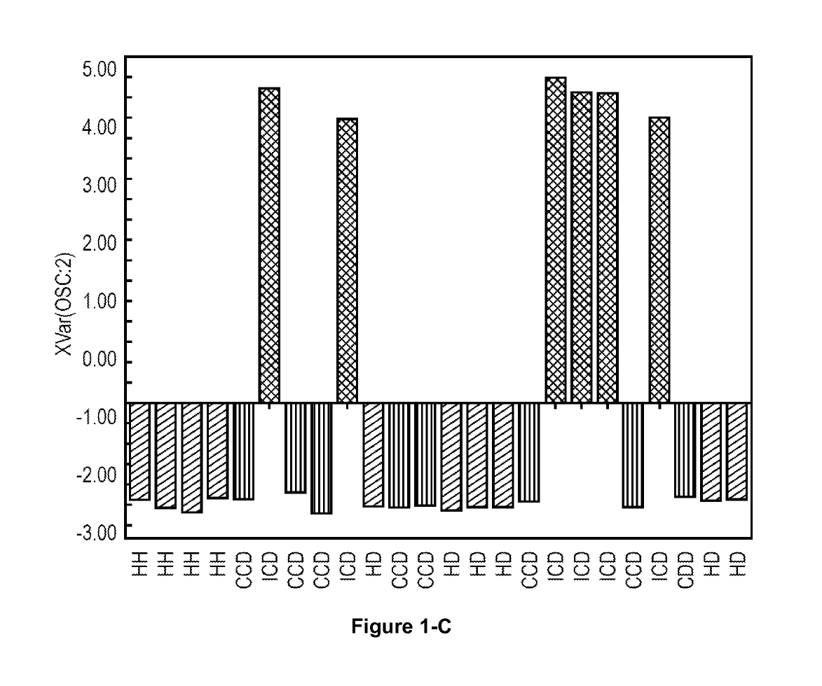

Using a partial least squares (PLS) statistical approach on corrected mass data the separation between disease phenotypes was even more pronounced than when using the PCA model, with a clear separation of individuals with ICD from those with CCD and from healthy individuals (FIG. 1A) and some examples of differentiating metabolites are shown in FIG. 1B. This differentiation according to disease phenotype that was seen using both the PCA and PLS approaches provides further support to the recent hypothesis that ICD and CCD are different disease phenotypes of CD. The outlier with CCD was the youngest of our Crohn's patients (born 1986) and had only had the disease for 4 years at the time of sampling, whereas all the others have had the disease for >10 years.

The 2nd component of the PLS model also revealed a clear separation, not only between the individuals with ICD versus CCD and healthy, but also between CCD and healthy. These data are the first that we have seen from this sample cohort that differentiate healthy from CCD individuals. Therefore, the resolution of separation of the groups was higher for the metabolite profiles than for the microbial community profiles [17], thus demonstrating the potential for use of metabolomics as an approach for accurate disease diagnosis. One reason for the higher discrimination of the metabolite data compared to the microbial data could be the direct link of metabolites to function since they represent the final signature of enzymatic processes occurring in the gut. By comparison, detection of microbial presence based on DNA targets may be misleading since many species in the gut may be dormant, dead, or transient and this information is not available when assessing DNA alone. Another explanation for the difference between DNA-based surveys and metabolite-based surveys could be that some microbes may be present and active in both diseased and healthy individuals, but may not have a significant effect on levels of the metabolites that we are screening for.

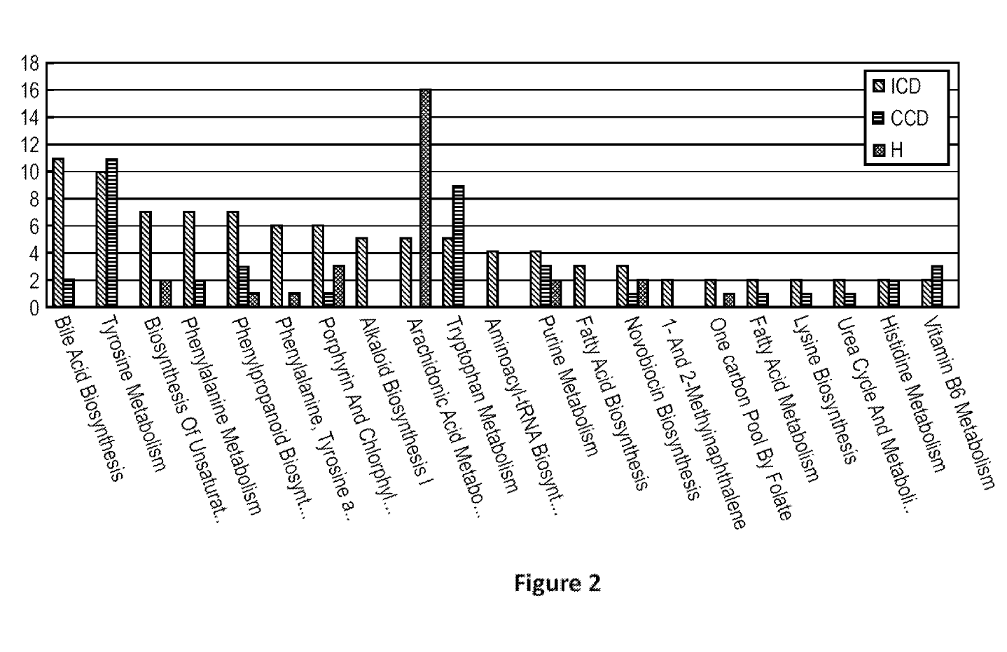

Metabolites within a broad range of pathways contributed to the differentiation of healthy from diseased individuals, as well as between disease phenotypes (FIG. 2A, Table 1). From the total number of 18706 measured masses, we found that 7919 were discriminating for a specific disease phenotype: 2155 for ICD, 3113 for CCD and 2650 for healthy.

Table 1 is as follows:

TABLE 1
Identified fecal metabolites that contribute to the discrimination of disease phenotypes.

| | | CCD | | Healthy | | ICD | | |
|---|---|---|---|---|---|---|---|---|
| | | | Detected | | Detected | | Detected | |
| Pathway | Metabolite | Mean ¹ | (n/8) | Mean ¹ | (n/10) | Mean ¹ | (n/6) | P |
| Tyrosine | 2-Carboxy-2,3-dihydro-5,6- | 202889263 ₐ * | 5 | 133363 _{b} | 1 | 2616539 _{b} | 4 | .015 |
| metabolism | dihydroxyindole/dopaquinone | | | | | | | |
| | 4-hydroxyphenyl-acetylglycine | 8699313 ₐ * | 5 | 0 _{b} | 0 | 140068 _{b} | 1 | .012 |
| | (Z)/4/hydroxyphenyl- | 502918 ₐ * | 4 | 0 _{b} | 0 | 0 _{b} | 0 | .006 |
| | acetaldehyde-oxime | | | | | | | |
| Amino acids | Tyrosine | 335531 _{b} | 3 | 86251 _{b} | 1 | 1441491 ₐ * | 6 | .001 |
| | Tryptophan | 208343 _{b} | 2 | 0 | 0 | 1087303 ₐ * | 5 | .001 |
| | Phenylalanine | 0 _{b} | 0 | 0 | 0 | 1405997 ₐ * | 5 | .001 |
| Bile acid | Glycocholate | 715048 _{b} * | 4 | 0 | 0 | 1847470 ₐ * | 5 | .001 |
| metabolism | Taurocholate | 312872 _{b} | 2 | 179560 _{b} | 2 | 14227172 ₐ * | 5 | .002 |
| | Trihydroxy-6β-cholanate | 20481784 _{b} | 8 | 16410877 _{b} | 10 | 1016307115 ₐ * | 6 | .001 |
| | Chenodeoxyglycocholate/ | 335746 _{b} | 2 | 83591 _{b} | 1 | 1200688 ₐ * | 5 | .002 |
| | Glycochenodeoxycholate | | | | | | | |
| Fatty acid | Oleic acid | 451036 _{b} | 2 | 580092 _{b} | 3 | 5745324 ₐ * | 5 | .010 |
| biosynthesis | Stearic acid | 661070 _{b} | 3 | 687268 _{b} | 5 | 2658962 ₐ * | 5 | .021 |
| | Palmitic acid | 357430 _{b} | 2 | 184491 _{b} | 2 | 2476141 ₐ * | 5 | .006 |
| | arachidonic acid | 0 _{b} | 0 | 0 _{b} | 0 | 715296 ₐ * | 3 | .004 |
| | octadecatrienoic acid | 0 _{b} | 0 | 0 _{b} | 0 | 568143 ₐ * | 3 | .005 |
| | linoleic acid | 920810 _{b} | 3 | 778285 _{b} | 4 | 5202328 ₐ * | 5 | .022 |
| Arachidonic | prostaglandin F2α | 607077 | 4 | 1400944 * | 9 | 977053 | 4 | .142 |
| acid | 2,3-dinor-8-iso- | 4002093 | 8 | 4048724 * | 10 | 1918427 | 6 | .093 |
| metabolism/ | prostaglandin F2α | | | | | | | |
| prostaglandins | prostaglandin F1α | 1216394 _{b} | 7 | 2482457 ₐ * | 10 | 812282 _{b} | 4 | .017 |
| | prostaglandin E2α | 1726834 | 8 | 2662085 * | 9 | 1077826 | 5 | .088 |
| Phenylalanine | 3-(4-hydroxy-phenyl)propionic | 2945178 _{a b} * | 4 | 513000 _{b} | 4 | 3632083 ₐ * | 6 | .001 |
| metabolism | acid/3-(4- | | | | | | | |
| | hydroxyphenyl)lactate | | | | | | | |

* Indicates the group discriminated by the given metabolite in PLS-DA; n, indicates number out of the total number of individuals in each disease category.
¹ Mean, refers to the mean amount (daltons) of the metabolite detected in all individuals within a given disease category.
P values indicate a significant difference between groups based on Anova; different subscript letters indicate groups that differed significantly (<.05).

Of the discriminating masses 13.3%, 9.5% and 9.3% for ICD, CCD and healthy groups, respectively, could be assigned to metabolic pathways using MassTRIX. However, 89.6% could not be assigned using MassTRIX software indicating that the available databases used for the identification of masses are yet limited by incompleteness. Some of the unassigned masses were interesting, such as the negative ion at mass-to-charge ratio (m/z) of 229.1557 or 391.2853 (see FIG. 1B), that were characteristic for ICD patients. Pathways with differentiating metabolites included those involved in the metabolism and or synthesis of amino acids, fatty acids, bile acids and arachidonic acid and these are discussed in more detail below.

We found numerous masses corresponding to metabolites within the tyrosine metabolic pathway (FIG. 2B) that discriminated diseased from healthy groups. For example, dopaquinone, an oxidation product of dopa and an intermediate in the formation of melanin from tyrosine, was more abundant (P<0.05) in CD patients (both CCD and ICD) than in healthy individuals. LDOPA has been observed at elevated levels in inflamed mucosa of IBD patients [18]. There is also some evidence that polymorphisms in the dopamine receptor D-2 play a role in CD [19]. In addition, 4-hydroxyphenylacetylglycine and (Z)-4-hydroxyphenylacetaldehyde-oxime contributed substantially to the separation of CCD individuals. In a previous study 4-hydroxyphenylacetylglycine in urine was negatively correlated to the abundance of Faecalibacterium prausnitzii in the gut [5]. By contrast we found higher levels of 4-hydroxyphenylacetylglycine levels in the feces of a subset of patients with elevated F. prausnitzii abundances previously published from the same samples [2]. These differences could be due to the different sample origins in the different studies; i.e. urine compared to feces. In our previous studies and others F. prausnitzii was more abundant in healthy individuals compared to individuals with Crohn's disease [2], [20]. These results suggest there could be a link between F. prausnitzii and the metabolite 4-hydroxyphenylacetylglycine that warrants further study.

Increased amounts of metabolites involved in tyrosine metabolism coincides with earlier reports of increased transcripts of genes involved in tyrosine metabolism in peripheral blood mononuclear cells from Crohn's patients [21]. However, it is not clear what (if any) role increased tyrosine metabolism may play in CD. Interestingly, protein tyrosine phosphatases have been associated with autoimmune diseases [22], although such mutations have not been correlated to CD.

The amino acids tryptophan and phenylalanine were also indicative of the ICD phenotype (Table 1). The presence of tryptophan in the feces might correspond to a subset of previously described CD patients with a specific depression of blood tryptophan levels [23]. Interestingly, one ICD individual did not have detectable levels of tryptophan or phenylalanine, and this individual also had the lowest abundance of mucosal E. coli [2], compared to the other ICD individuals. Although other organisms could also be correlated to these metabolites, it would be of interest to further investigate any links between E. coli abundance and amino acids in the gut. Previous studies [24] have shown that E. coli isolated from CD patients have pathogen-like behavior in vitro, and may play a role in the inflammatory process. Marchesi et al. [7] also observed increased levels of some other amino acids in fecal samples of CD patients with active disease (ours were in remission), but these were different amino acids than those we found; i.e. alanine, isolueucine, leucine and lysine. The presence of amino acids in the feces of ICD patients in general may be the result of malabsorption resulting from the shortening of the small bowel or due to subclinical inflammation, or conversely the result of secretion into the bowel. Regardless of the underlying mechanism, these results suggest that a special consideration of amino acid balance should be made for patients with active disease as well as those in remission.

Many masses corresponding to metabolites within the bile acid biosynthesis pathway contributed to the segregation of disease phenotypes. In particular, the mass corresponding to glycocholate was prevalent in a majority of individuals with CD (Table 1), but not detected in healthy individuals. Masses corresponding to taurocholate, 3a, 7a, 12a-trihydroxy-5β-cholanate and chenodeoxyglycocholate were also particularly high (P<0.001) in ICD patients (Table 1). The majority of bile acids are reabsorbed in the distal ileum, largely accomplished by an apical sodium dependent bile acid transporter, and thus ileal resection could explain the reduced bile acid absorption in ICD patients. However, only 1 of the 4 CCD patients with glycocholate in the feces had undergone resection surgery. This is consistent with the finding that patients with and without ileal resection have altered bile composition [25]. Increased bile in the feces may indicate that although these subjects were in remission they were nevertheless experiencing subclinical inflammation as bile acid absorption has been shown to be inhibited by inflammation [26], or increased mucosal permeability [27]. A detrimental feedback loop could be created where inflammation results in reduced bile absorption, therefore increased bile in the lumen, which in turn causes increased inflammation.

Masses corresponding to both saturated and unsaturated fatty acids, including oleic acid, stearic acid, palmitic acid, 6Z-, 9Z-, and 12Z-octadecatrienoic acid, linoleic acid and arachadonic acid, were also higher in patients with ICD compared to the other groups (Table 1). Fernandez Baneres et al. [28] previously reported elevated levels of arachidonic acid and linoleic acid in colonic mucosa of CD patients, consistent with our results from fecal samples, but they also found reduced amounts of oleic acid in the mucosa, contrary to the increased levels we observed.

Arachidonic acid is particularly interesting because it is known to mediate inflammation and the functioning of several organs and systems either directly or upon its conversion into eicosanoids. Arachidonic acid has previously been shown to increase the expression of the intracellular adhesion molecule (ICAM)-1, which is involved in the recruitment of leukocytes, suggesting another role of this molecule [29]. Linoleic acid and arachidonic acid are also essential for the synthesis of prostaglandins (PG), which are important immune signaling molecules. However, in our study the increased abundances of these fatty acids did not correspond to levels of PG (see below) and further studies are necessary to determine their possible links and correlations to CD.

Masses corresponding to PG and their breakdown products including PGF2a and 2,3dinor-8-iso-PGF2a, thromboxane/6-Keto-PGF1a/PG12 and PGE2 were more prevalent in healthy individuals (including the young group) than those with CCD and ICD (Table 1). The role of PG as immune signaling molecules is particularly interesting as CD is associated with disregulated immune function. CD-associated alleles have recently been negatively correlated to quantitative expression levels of prostaglandin receptor EP4 (PTGER4) [30] and PTGER4 knockout mice experience more severe colitis in the dextran sodium sulfate model of colitis. The reduced levels of PGs that we observed may reflect a reduced absorption of their precursors, arachidonic acid and linoleic acid, as indicated above. While we observed reduced prostaglandin levels in CD patients that were in remission, PG particularly PGE2, have been observed to be more abundant in patients with active CD [31]. This is consistent with the fact that PGE2 is proinflammatory, acting through the EP-2/4 receptor on dendritic cells inducing the expression of IL-23 resulting in a TH 17 phenotype associated with CD [31]. Therefore, reduced PG levels in CD patients in remission observed here may make them susceptible to relapse, although elevated levels during active disease could be a cause of tissue damage. Intriguingly, 3-(4hydroxyphenyl)-propionic acid, that has previously been shown to suppress PGE2 production [32], was particularly elevated in a subset of CCD and ICD individuals.

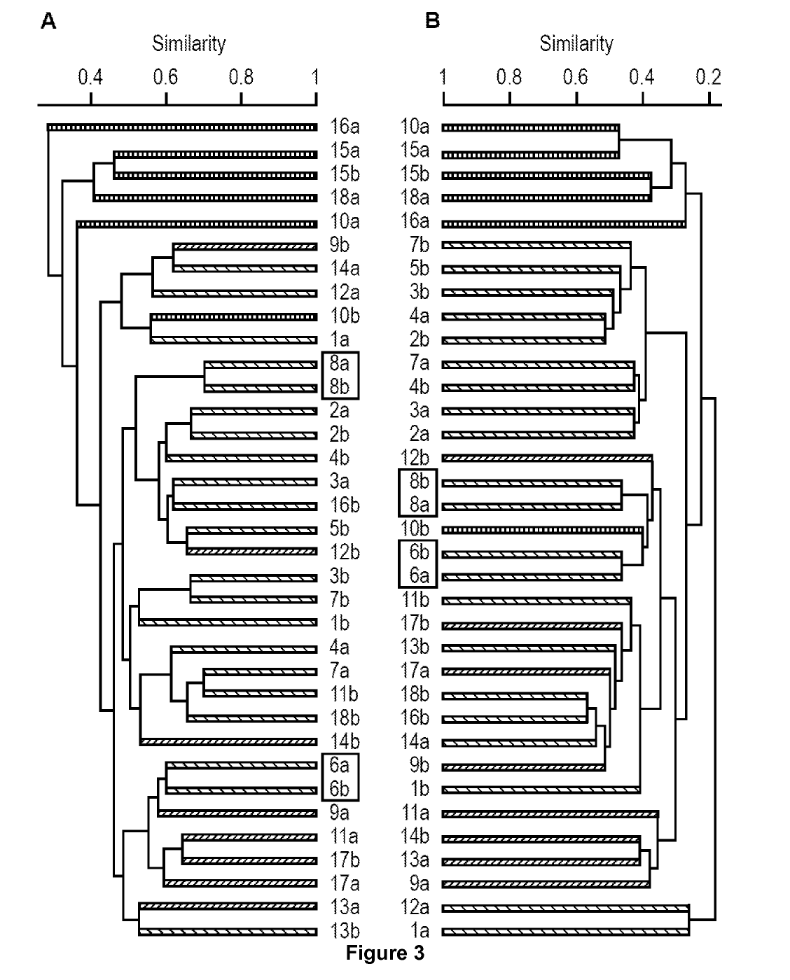

Manhattan distances were calculated from the metabolic profiles of all individuals to determine whether the gut metabolomes of twins were more similar to each other than to unrelated individuals. The inter-twin similarity (mean±SE) of healthy twins (0.513±0.035) and concordant twins (0.431±0.070) was greater (P<0.001) than that of discordant twins (0.276±0.048). This coincides with the reduced similarity of microbial profiles previously observed in the discordant twins [17]. A correlation between the metabolic and microbial distance matrices (r=0.348, P<0.001) coincides with findings from Li et al. [5] correlating fecal microbial profiles to urinary metabolites, indicating a contribution of bacteria to overall metabolic profiles in the human host. We observed a stronger correlation between metabolic and microbial similarities when making within twin comparisons (r=0.748, P<0.001) strengthening the hypothesis that genetics plays a role in the formation and maintenance of the intestinal microbiome (FIG. 3). The most striking observation from the cluster analysis (FIG. 3) was the similar division of clusters according to the disease phenotype for both the microbial and metabolite data reinforcing the link between microbial community structure, function and disease.

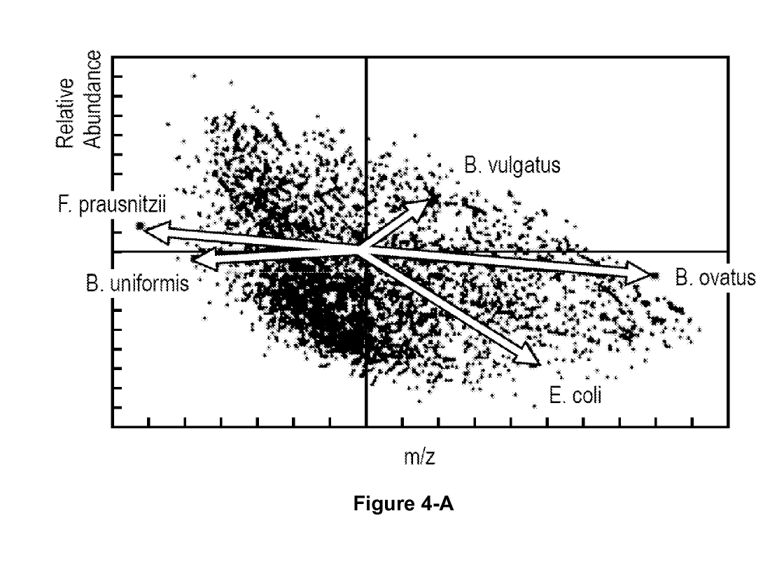

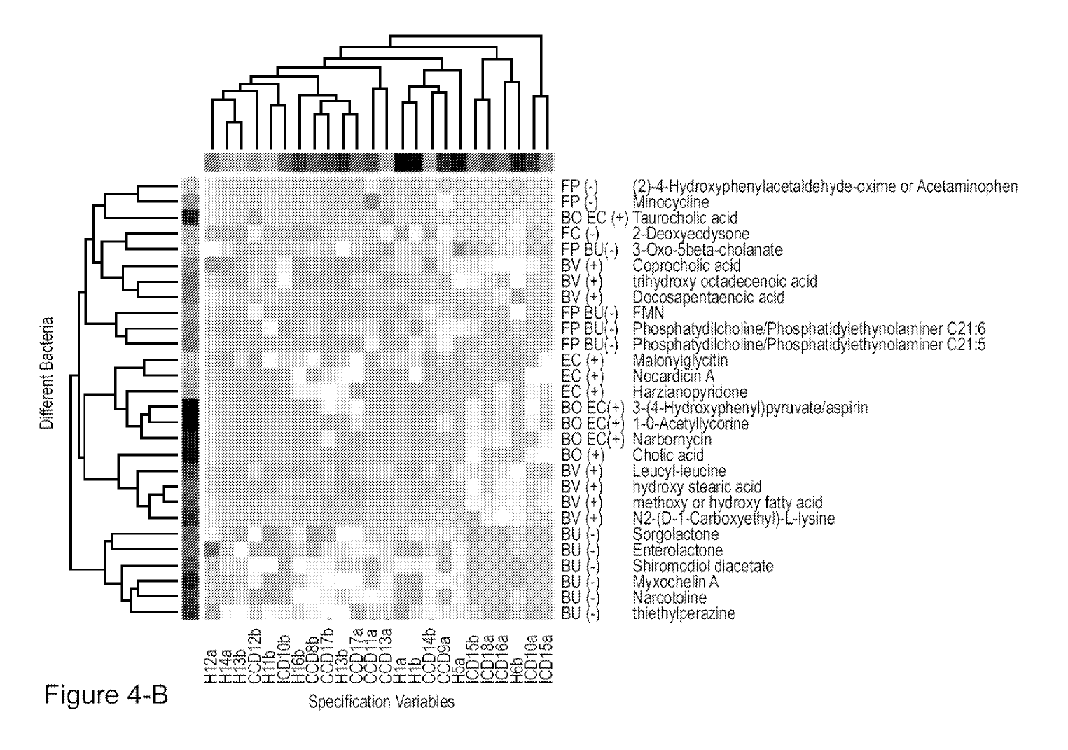

Subsequently, we also correlated the metabolomes to the relative abundances of specific bacterial populations within the same samples. We used a PLS model with relative abundances of specific microbial populations as the Y matrix and the m/z data as the independent variables to correlate the MS data to microbes and disease status (FIG. 4A). The masses were correlated to predefined key species that we previously found to be significantly more or less abundant depending on the disease phenotype of the host [2], [17]. For example, Bacteroides vulgatus (BV), B. ovatus (BO) and E. coli (EC) were present at significantly higher levels in the ICD group, whereas F. prausnitzii (FP) and B. uniformis (BU) were more abundant in the healthy and the CCD groups. The 10 masses with the highest regression coefficient value for each of the 5 bacteria indicated above were assigned in MassTRIX. The correlation coefficients were computed using bacterial and MS abundances and these data were expressed in a heat map for visualization of the data clustering (FIG. 4B). In this analysis the individuals clustered mainly into two groups: 1) ICD and 2) healthy+CCD, similar to what we found with the PCA plot of the original metabolite data (Fig. S1). Bacteria that were more abundant in individuals with ICD (BV, BO and EC) were those that were most strongly correlated to bile acids, including taurocholic and cholic acids, and fatty acids, including stearic, and docosapentanoic acids.

Conversely, bacteria that were more abundant in healthy or CD phenotypes (BU and FP) were correlated to phospholipids and flavin mononucleotide (FMN). The correlation of these metabolites to specific bacterial groups merits further attention, such as the causality of the relationship between, E. coli and elevated levels of taurocholic acid. It should be kept in mind, however, that other bacteria that weren't specifically included in this screening could be contributing to the metabolite profiles seen.

In summary, this study demonstrates the potential of metabolomics to provide a means to differentiate disease phenotypes and to give new insights into the etiology of Crohn's disease. Our study also emphasizes the importance of metabolites produced by the gut microbiota for a healthy gut environment. The analysis procedure was rapid to perform and resulted in highly accurate mass data. Several masses were found that differentiated healthy, ICD and CCD individuals. Interestingly, the similarity of metabolite profiles in healthy monozygotic twin pairs underscores the importance of genetics in determining the nature of the gut environment, including the bacterial species that are most dominant and the metabolites they produce. Further investigation of those masses that were important in differentiation of disease phenotypes, but that could not be assigned to structures, will be an aim for the future using structure identification tools involving hyphenated multidimensional separation, spectrometric and spectroscopic tools.

Our results pinpoint significant differences in the types and number of metabolites within specific pathways, including tyrosine and phenylalanine metabolism and bile acid and fatty acid biosynthesis that could be of key importance for different Crohn's Disease etiologies. Thus, not only the identified individual metabolites, but also the pathways they belong to, could lead to future therapeutic biomarkers or drug targets.

While the present invention has been described with reference to the specific embodiments thereof, it should be understood by those skilled in the art that various changes may be made and equivalents may be substituted without departing from the true spirit and scope of the invention. In addition, many modifications may be made to adapt a particular situation, material, composition of matter, process, process step or steps, to the objective, spirit and scope of the present invention. All such modifications are intended to be within the scope of the claims appended hereto.

What is claimed is:

1. A method for identifying a Crohn's disease (CD) phenotype of a subject in need of such identification comprising: identifying one or more biomarkers as described in Tables 1-4 in a fecal sample collected from a subject.

2. The method of claim 1, wherein said identifying comprises identifying one or more biomarkers as described in Table 1 in a fecal sample collected from the subject.

3. The method of claim 1, wherein said identifying comprises identifying one or more biomarkers as described in Table 2 in a fecal sample collected from the subject, wherein the presence of elevated or increased levels of one or more biomarkers in the feces of the subject described in Table 2 indicates that the subject is likely to suffer from colonic Crohn's disease (CCD).

4. The method of claim 1, wherein said identifying comprises identifying one or more biomarkers as described in Table 3 in a fecal sample collected from the subject, wherein the presence of elevated or increased levels of one or more biomarkers in the feces of the subject described in Table 3 indicates that the subject is likely to suffer from ileal Crohn's disease (ICD).

5. The method of claim 1, wherein said identifying comprises identifying one or more biomarkers as described in Table 4 in a fecal sample collected from the subject, wherein the presence of elevated or increased levels of one or more biomarkers in the feces of the subject described in Table 4 indicates that the subject is likely not suffering from Crohn's disease (CD).

6. The method of claim 1, wherein said identifying step comprises determining the mass-to-charge ratio (m/z) value of one or more metabolite in the fecal sample, and determining whether the m/z value of the metabolite is found in one of Tables 1-4.

7. The method of claim 1, further comprising: collecting the fecal sample from the subject, where the collecting step occurs prior to the identifying step.

8. The method of claim 1, wherein the subject is a human subject who has Crohn's disease (CD), is diagnosed with CD, is suspected to have CD, or is likely to have CD.

9. The method of claim 1, further comprising: providing a warning to the subject that the subject is suffering from CD, ICD or CCD.

10. The method of claim 1, further comprising: altering treatment of the subject so that the subject is treated for CD, ICD or CCD.

11. The method of claim 1, further comprising treating the subject for CD, ICD or CCD.

12. The method of claim 10, wherein treating the subject for CD, ICD or CCD comprises: administering a therapeutically effective amount of a drug suitable for treating or ameliorating at least one symptom of CD to the subject, or performing surgery on the subject.
